(19) United States
(12) Patent Application Publication
(10) Pub. No.: US 2016/0348103 A1
(43) Pub. Date: Dec. 1, 2016

(54) Oligonucleotides and Methods for Treatment of Cardiomyopathy Using RNA Interference

(71) Applicant: THE BOARD OF TRUSTEE OF THE LELAND STANFORD JUNIOR UNIVERSITY, Palo Alto, CA (US)

(72) Inventors: Matthew WHEELER, Sunnyvale, CA (US); Euan A. ASHLEY, Menlo Park, CA (US); Katheia M. ZALETA-RIVERA, Stanford, CA (US)

(73) Assignee: The Board of Trustees of the Leland Stanford Junior University, Stanford, CA (US)

(21) Appl. No.: 15/114,063

(22) PCT Filed: Jan. 26, 2015
(86) PCT No.: PCT/US2015/012966

§ 371 Date: Jul. 25, 2016

Related U.S. Application Data
(60) Provisional application No. 61/931,690, filed on Jan. 27, 2014.

(51) Int. Cl. C12N 15/113

(52) U.S. Cl. CPC C12N 15/113; C12N 2310/14

(57) ABSTRACT

Compositions and methods for treating cardiomyopathy using RNA interference are disclosed. In particular, embodiments of the invention relate to the use of oligonucleotides for treatment of cardiomyopathy, including small interfering RNAs (siRNAs) and short hairpin RNAs (shRNAs) that silence expression of disease-causing mutant alleles, such as the myosin MYL2 allele encoding human regulatory light chain (hRLC)-N47K and the MYH7 allele encoding human myosin heavy chain (hMHC)-R403Q while retaining expression of the corresponding wild-type allele.

### CROSS-REFERENCE TO RELATED APPLICATIONS

This application is a national stage of Application No. PCT/US2015/012966, filed Jan. 26, 2015, which application claims priority to U.S. Provisional Application No. 61/931,690 filed Jan. 27, 2014, the disclosures of which are incorporated herein by reference in their entireties.

### STATEMENT OF GOVERNMENT SPONSORED SUPPORT

This invention was made with Government support under contract OD006511 awarded by the National Institutes of Health. The Government has certain rights in this invention.

### FIELD OF THE INVENTION

The present invention pertains generally to compositions and methods for treating cardiomyopathy using RNA interference (RNAi). In particular, the invention relates to the use of oligonucleotides, including small interfering RNAs (siRNAs) and short hairpin RNAs (shRNAs) that preferentially silence expression of mutant alleles of human regulatory light chain (hRLC) and human myosin heavy chain (hMHC) for treatment of cardiomyopathy.

### BACKGROUND OF THE INVENTION

Cardiomyopathy is a genetic disease of the heart muscle and the most common cause of sudden death in young people and athletes. It is caused by heterozygotic missense mutations in genes encoding proteins of the cardiac sarcomere. To date, more than 400 mutations in over nine disease genes have been described.

Cardiomyopathy has been linked to a number of single nucleotide variants (SNVs) in the sarcomeric protein myosin. Serving as the molecular motor of heart cells, myosin generates mechanical force by ATP hydrolysis. It is a hexameric protein complex composed of two myosin heavy chains (β-MHC) encoded by the MYH7 gene, and four light chains, including two regulatory light chains (RLC) and two essential light chains (ELC) encoded by the MYL2 and MYL3 genes, respectively. Single nucleotide variants (SNVs) in the catalytic domains, calcium binding domains and phosphorylation sites of these myosin proteins alter the mechanical forces and the electrical signals necessary for balancing the cardiac cells and tissue structure.

Medical therapy for cardiomyopathy remains largely palliative. Beta-blockers, calcium channel blockers, and disopyramide are the mainstay of pharmacological management, but effects are modest and often limited by side effects.

### SUMMARY OF THE INVENTION

Gene silencing technology is important drug discovery and represents a novel therapeutic approach that allows targeting of the genetic causes of disease and selective downregulation of expression of pathogenic mutant alleles while sparing expression of the coincident normal alleles.

Embodiments of the present invention directly target alleles mutated in dominantly inherited forms of cardiovascular disease in order to reduce expression and translation of the mutant transcript and favor expression of the normal transcript and alleviate signs and symptoms of disease. Advantageously, allele-specific targeting allows for direct treatment of underlying disease mechanism in patients with inherited cardiomyopathies. Reduction in expression of mutated alleles in a site-specific manner promotes normal allele expression ratio and will facilitate normalization of protein and myocyte function. Methods and techniques for developing specific cardiac targeting therapeutics are described. Other embodiments of the present invention include methods for identifying candidate siRNA and shRNA. For example, embodiments of the present invention include methods and techniques for designing of candidate shRNAs that can be advantageously used in certain embodiments of the present invention.

Embodiments of the present invention generally relate to compositions and methods for treating cardiomyopathy using RNA interference. Embodiments of the invention relate to the use of RNAi oligonucleotides, including small interfering RNAs (siRNAs) and short hairpin RNAs (shRNAs), for selective downregulation of human regulatory light chain (hRLC) and human myosin heavy chain (hMHC) variants for treatment of cardiomyopathy.

In an embodiment, two families of silencing constructs were generated. Another embodiment of the present invention is a method for identifying and generating a series of vectors capable of treating inherited cardiovascular diseases. Exemplary constructs are shown that specifically target mutations responsible for hypertrophic cardiomyopathy. In an embodiment, silencing constructs of the siRNA type were generated targeting a single base pair mutation in the MYL2 gene at position 47 of the protein.

Mismatch position (e.g., mutant vs. normal allele mismatch) in mature siRNA sequence at position 6, 7, 8 and 10 are shown to be effective in producing differential silencing according to an embodiment of the present invention. In an embodiment, the constructs are packaged into replication deficient viral vectors and delivered in vivo via venous or direct injection into targeted tissue with or without addition of a immune modulating agent.

An embodiment of the invention includes an RNA interference (RNAi) oligonucleotide that selectively downregulates expression of a mutant human myosin, MYH7 or MYL2, allele associated with cardiomyopathy. In an embodiment, allele selective silencing is achieved by use of one or more RNAi oligonucleotides that selectively downregulate expression of a target mRNA encoding a particular myosin heavy chain or regulatory light chain variant while allowing expression of the wild-type allele. RNAi oligonucleotides act, for example, by binding to and reducing translation or increasing degradation of the target mRNA. In embodiments of the present invention, RNAi oligonucleotides are typically 19 to 55 nucleotides in length and may comprise a sense strand and an antisense strand that is sufficiently complementary to hybridize to the sense strand. One or more nucleotides of the RNAi oligonucleotide may be modified to improve, for example, the stability of the RNAi oligonucleotide, its delivery to a cell or tissue, or its potency in triggering RNAi. In an embodiment, the RNAi oligonucleotide comprises one or more nucleotides comprising 2′-O-methyl modifications. For example, an RNAi oligonucleotide may comprise a 2-O-methyl modification at every third nucleotide. Additionally, RNAi oligonucleotides may further comprise a nucleotide overhang at the 3′ end or 5′ end of the sense strand or antisense strand. In one embodiment, the antisense strand further comprises a phosphate group at the 5′ end. In another embodiment, the antisense strand further comprises a nucleotide addition or substitution of uridine at the 3′ end. The RNAi oligonucleotide may further comprise a detectable label.

In certain embodiments, the RNAi oligonucleotide is an siRNA or an shRNA. Double-stranded siRNAs typically comprise a sense strand and an antisense strand, each typically 19 to 29 nucleotides in length, in certain embodiments. The sense strand and the antisense strand can be connected by a loop to form an shRNA. The loop of an shRNA may be any size but is typically 3 to 12 nucleotides in length and may further comprise a restriction site. In certain embodiments, the loop consists of the sequence of CAAGCTTC or the sequence of SEQ ID NO:1.

Embodiments of the invention includes an RNAi oligonucleotide that selectively downregulates expression of a regulatory light chain variant comprising a lysine substitution at position 47 (RLC-47K), wherein the RNAi oligonucleotide comprises:
a) a sense strand comprising a sequence selected from the group consisting of SEQ ID NOS:10-12 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the RNAi oligonucleotide reduces expression of the RLC-47K; and
b) an antisense strand comprising a region that is complementary to the sense strand.

In an embodiment, the RNAi oligonucleotide is an siRNA that selectively downregulates expression of RLC-47K selected from the group consisting of:
a) an siRNA comprising a sense strand comprising the sequence of SEQ ID NO:10 and an antisense strand comprising the sequence of SEQ ID NO:25;
b) an siRNA comprising a sense strand comprising the sequence of SEQ ID NO:26 and an antisense strand comprising the sequence of SEQ ID NO:27;
c) an siRNA comprising a sense strand comprising the sequence of SEQ ID NO:31 and an antisense strand comprising the sequence of SEQ ID NO:32;
d) an siRNA comprising a sense strand comprising the sequence of SEQ ID NO:10 and an antisense strand comprising the sequence of SEQ ID NO:27;
e) an siRNA comprising a sense strand comprising the sequence of SEQ ID NO:11 and an antisense strand comprising the sequence of SEQ ID NO:92;
f) an siRNA comprising a sense strand comprising the sequence of SEQ ID NO:12 and an antisense strand comprising the sequence of SEQ ID NO:93;
g) an siRNA comprising a sense strand comprising the sequence of SEQ ID NO:106 and an antisense strand comprising the sequence of SEQ ID NO:107;
h) an siRNA comprising a sense strand comprising the sequence of SEQ ID NO:108 and an antisense strand comprising the sequence of SEQ ID NO:109;
i) an siRNA comprising a sense strand comprising the sequence of SEQ ID NO:110 and an antisense strand comprising the sequence of SEQ ID NO:111;
j) an siRNA comprising a sense strand comprising the sequence of SEQ ID NO:112 and an antisense strand comprising the sequence of SEQ ID NO:113;
k) an siRNA comprising a sense strand comprising the sequence of SEQ ID NO:114 and an antisense strand comprising the sequence of SEQ ID NO:115;
l) an siRNA comprising a sense strand comprising the sequence of SEQ ID NO:116 and an antisense strand comprising the sequence of SEQ ID NO:117; and
m) an siRNA comprising a sense strand comprising the sequence of SEQ ID NO:118 and an antisense strand comprising the sequence of SEQ ID NO:119.
In another embodiment, the RNAi oligonucleotide is an shRNA that selectively downregulates expression of RLC-47K comprising a sequence selected from the group consisting of SEQ ID NOS:35-37.

Other embodiments of the present invention includes an RNAi oligonucleotide that selectively downregulates expression of a myosin heavy chain variant comprising a glutamine substitution at position 403 (MHC-403Q), wherein the RNAi oligonucleotide comprises:
a) a sense strand comprising a sequence selected from the group consisting of SEQ ID NO:53 and SEQ ID NO:54 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the RNAi oligonucleotide reduces expression of the MHC-403Q; and
b) an antisense strand comprising a region that is complementary to the sense strand. In one embodiment, the RNAi oligonucleotide is an shRNA that selectively downregulates expression of MHC-403Q comprising a sequence selected from the group consisting of SEQ ID NO:64 and SEQ ID NO:65.

Another embodiment of the present invention includes a recombinant polynucleotide comprising a promoter operably linked to at least one polynucleotide encoding an RNAi oligonucleotide (e.g., siRNA or shRNA) described herein. In an embodiment, the recombinant polynucleotide comprises a first polynucleotide sequence encoding the sense strand of an siRNA and a second polynucleotide sequence encoding the antisense strand of an siRNA. In another embodiment, the recombinant polynucleotide comprises a polynucleotide sequence encoding an shRNA, including the sense sequence, antisense sequence, and hairpin loop of the shRNA. The recombinant polynucleotide may comprise an expression vector, for example, a bacterial plasmid vector or a viral expression vector, such as, but not limited to, an adeno-associated virus, adenovirus, retrovirus (e.g., γ-retrovirus and lentivirus), poxvirus, baculovirus, or herpes simplex virus vector. In certain embodiments, the viral vector is a replication deficient viral vector. In an embodiment, the viral vector is an adeno-associated virus-9 (AAV-9) vector. In another embodiment, the viral vector comprises a sequence selected from the group consisting of SEQ ID NOS:121-123. Exemplary sequences of constructs comprising an expression vector encoding an shRNA are shown in SEQ ID NO:120, SEQ ID NO:124, and SEQ ID NO:125.

Another embodiment of the present invention includes a composition comprising one or more RNAi oligonucleotides (e.g., siRNAs or shRNAs) and/or recombinant polynucleotides or vectors encoding one or more RNAi oligonucleotides described herein. The composition may further comprise a pharmaceutically acceptable carrier. In addition, the composition may further comprise one or more other agents for treating cardiomyopathy. Compositions may be administered to a subject by any suitable method, including but not limited to, intracardially, intramyocardially, intraventricularly, intravenously, or intra-arterially.

Another embodiment of the invention includes a method for treating a subject having cardiomyopathy by administering a therapeutically effective amount of a composition comprising one or more RNAi oligonucleotides and/or recombinant polynucleotides encoding one or more RNAi oligonucleotides to the subject. Cardiomyopathies that can be treated by methods of the present invention include, but are not limited to, dilated cardiomyopathy, hypertrophic cardiomyopathy, restrictive cardiomyopathy, arrhythmogenic right ventricular cardiomyopathy, and left ventricular noncompaction cardiomyopathy.

In an embodiment, a subject undergoing treatment has been shown by genotyping to have the MYH7 allele encoding myosin heavy chain (MHC)-403Q and is administered a composition comprising one or more RNAi oligonucleotides or recombinant polynucleotides encoding one or more RNAi oligonucleotides that selectively downregulate expression of MHC-403Q. In another embodiment, a subject undergoing treatment has been shown by genotyping to have the MYL2 allele encoding regulatory light chain (RLC)-47K and is administered a composition comprises one or more RNAi oligonucleotides or recombinant polynucleotides encoding one or more RNAi oligonucleotides that selectively downregulate expression of RLC-47K, said RNAi oligonucleotides.

In an embodiment, an effective amount of an RNAi oligonucleotide (e.g., siRNA or shRNA) or a recombinant polynucleotide or vector encoding an RNAi oligonucleotide is an amount sufficient to downregulate expression of a target mRNA or protein (e.g., human myosin MYH7 allele encoding MHC-403Q or MYL2 allele encoding RLC-47K) and can be administered to a subject in one or more administrations, applications, or dosages. By therapeutically effective dose or amount of an RNAi oligonucleotide or a recombinant polynucleotide or vector encoding an RNAi oligonucleotide is intended an amount that, when administered, as described herein, brings about a positive therapeutic response, such as improved recovery from cardiomyopathy. Improved recovery may include a reduction in one or more cardiac symptoms, such as dyspnea, chest pain, heart palpitations, lightheadedness, or syncope. Additionally, a therapeutically effective dose or amount of an RNAi oligonucleotide may improve cardiomyocyte contractile strength and sarcomere alignment.

Another embodiment of the invention includes a method of downregulating expression of RLC-47K or MHC-403Q in a subject, the method comprising: administering an effective amount of at least one RNAi oligonucleotide (e.g., siRNA or an shRNA) described herein to the subject.

Another embodiment of the invention includes a method of downregulating expression of RLC-47K or MHC-403Q in a cardiac cell, the method comprising introducing an effective amount of an RNAi oligonucleotide (e.g., siRNA or an shRNA) described herein into the cell. In one embodiment, the cardiac cell is a cardiomyocyte.

Another embodiment of the invention includes a method for selectively decreasing the amount of a RLC-47K or MHC-403Q protein in a cardiac cell of a subject, the method comprising introducing an effective amount of an RNAi oligonucleotide (e.g., siRNA or an shRNA) described herein into the cardiac cell of the subject.

Another embodiment of the present the invention includes a kit comprising one or more RNAi oligonucleotides described herein or recombinant polynucleotides or vectors encoding them and instructions for treating cardiomyopathy. In certain embodiments, the kit comprises one or more RNAi oligonucleotides (e.g., siRNAs or shRNAs) or recombinant polynucleotides or vectors encoding RNAi oligonucleotides that selectively downregulate expression of the human MYH7 allele encoding MHC-403Q or the human MYL2 allele encoding RLC-47K, or a combination thereof. One or more RNAi oligonucleotides and/or recombinant polynucleotides or vectors encoding them may be combined in a pharmaceutical composition. The kit may further comprise means for delivering the composition to a subject.

These and other embodiments and advantages can be more fully appreciated upon an understanding of the detailed description of the invention as disclosed below in conjunction with the attached Figures.

### BRIEF DESCRIPTION OF THE DRAWINGS

The following drawings will be used to more fully describe embodiments of the present invention.

FIG. 1 shows small interference RNAs (siRNAs) sequences (WT and M2-M19, SEQ ID NOS:6-24) designed to target the single nucleotide variant “A” (SNV-A) highlighted in gray on the mutant MYL2-47K allele according to an embodiment of the present invention. The target mRNA sequences for wild type MYL2-47N and mutant MYL2-K alleles (SEQ ID NOS:2-5) are also shown.

FIG. 2 shows siRNAs of W16 and M2-M19 (SEQ ID NOS:6-24, SEQ ID NO:27, and SEQ ID NOS:88-105. Underscored nucleotides contain methyl groups. Antisense strands contain three nucleotide overhangs at the 3′ end and a phosphate group at the 5′ end according to an embodiment of the present invention.

FIGS. 3A and 3B show relative expression of wild-type MYL2-47N and mutant MYL2-N47K in the presence of different siRNAs according to embodiments of the present invention. FIG. 3A shows sequences of siRNAs M20-M25 (SEQ ID NOS:25-34). Underscored nucleotides contain methyl groups. Antisense strands contain deoxy-thymidine overhangs at the 3′ end and a phosphate group at the 5′ end according to an embodiment of the present invention. The SNV is highlighted. FIG. 3B shows results for siRNAs M5-M7 and M20-M25 according to an embodiment of the present invention.

FIG. 4 shows different modifications of the M7 siRNA (SEQ ID NOS:106-119). Underscored nucleotides contain methyl groups. Antisense strands contain three nucleotide overhangs at the 3′ end and a phosphate group at the 5′ end according to an embodiment of the present invention. The SNV is highlighted. Each siRNA contains non-pair Watson-crick modifications.

FIG. 5 shows the design and cloning of shRNAs in the AAV9 vector pAAV-H1p RSV_{P}-Cerulean.

FIG. 6 shows fluorescence activated cell sorting (FACS) of stable transfected HEK cells with MYL2-47N-GFP and MYL2-47K-mCherry and transfected with plasmid expressing shRNAs: M5.8L, M6.8L and M7.8L.

FIGS. 7A and 7B show the design of quantitative polymerase chain reaction (q-PCR) assays using a blocker for allele discrimination. FIG. 7A shows amplification of the mutant with blocker (B1) and with no blocker (NB) using wild type (WT) and mutant template. FIG. 7B shows amplification of the wild type using different blockers (B3, B4 and B5) and with no blocker (NB) and WT and mutant template.

FIG. 8 shows relative mRNA quantification using qPCR (q-PCR system from FIGS. 7-8 of stable transfected HEK cells with plasmids MYL2-47N-GFP and MYL2-47K-mCherry and treated with plasmids expressing shRNAs: M5.8L, M6.8L and M7.8L.

FIG. 9 shows relative SNP quantification using pyrosequencing of stable transfected HEK cells with plasmids MYL2-47N-GFP and MYL2-47K-mCherry and treated with plasmids expressing shRNAs: M5.8L, M6.8L and M7.8L.

FIG. 10 shows genotype determination of human MYL2-N47K mouse model by PCR and Bgl II restriction digestion.

FIG. 11 shows allele quantitative PCR of transgenic neonatal cardiomyocyte cells (NCM) transduced with AAV9 expressing M7.8L shRNA.

FIGS. 12A-12C show micropatterning of neonatal cardiomyocytes cultured on a micro stamp. FIG. 12A shows human transgenic neonatal cardiomyocytes transduced with AAV9 expressing M7.8L shRNA and cerulean reporter. FIG. 12B shows that NCM have an elongated shape and sarcomeric organization after cultured on a micro stamp. FIG. 12C shows an image of NCM cultured on a stamp and fixed and stained against alpha-actinin and DNA.

FIG. 13 shows contractile studies of elongated cardiomyocytes.

FIG. 14 shows siRNAs sequences (H1-H19, SEQ ID NOS:44-62) designed to target the single nucleotide variant “A” (SNV-A) of the human MYH7-R403Q allele mutant. The target mRNA sequences for wild type MYH7-403R and mutant MYH7-403Q alleles (SEQ ID NOS:40-43) are also shown.

FIG. 15 shows fluorescence activated cell sorting of the relative GFP and mCherry expression of double stable transfected human embryonic kidney cells containing MYH7-403R-GFP and MYH7-403Q-mCherry and transfected with H10.8L and H11.8L shRNAs.

FIG. 16 shows relative SNP quantification using pyrosequencing of stable transfected HEK cells with plasmids MYh7-403R-GFP and MYH7-403Q-mCherry and treated with plasmids expressing shRNAs: H10.8L and H11.8L.

FIG. 17 shows relative mRNA quantification of hMYH7 and hMYH6 of human R403Q cardiomyocytes differentiated from induced pluripotent cells (iPSc) and transduced with AAV9 expressing H10.8L and H11.8L shRNAs.

FIGS. 18A and 18B show AAV9-Luciferase viral vectors expressing M7.8L shRNA under an H1 promoter for in vivo experiments in mice containing human MYL2 wild type and mutant transgenes. FIG. 18A shows a schematic of the pAAV-RSV-eGFP-T2A-Fluc2 vector (SEQ ID NO:123). FIG. 18B shows a schematic of the pAAV-CBA-Fluc vector (SEQ ID NO:122).

FIGS. 19A-F show information relating to position seven in siRNA and shRNA allele specific silenced MYL2-47K mutation in a HEK293 cell model stably transfected with GFP fused to the human MYL2-47N normal allele and mCherry fused to the human MYL2-47K mutated allele. FIG. 19A shows protein quantification of Green and mCherry reporters using Fluorescence activated cell sorting (FACS) after transfection with different siRNAs targeting the MYL2-N47K mutation. FIG. 19B shows protein quantification of Green and mCherry reporters using FACS after transfection with chemical modified siRNAs M5, M6 and M7. FIG. 19C shows protein level quantification of green and mCherry fluorescent reporters 62 h after transfection with plasmids expressing shRNAs M5.8L, M6.8L and M7.8L. FIG. 19D shows mRNA level quantification of the human normal and mutated alleles using quantitative PCR and specific blockers. FIG. 19E shows single nucleotide quantification of the normal ‘C’ and variant ‘A’ using pyrosequencing. CTRL=double transfected HEK cells with plasmids, MYL2-47N or normal allele fused to Green and MYL2-47K or mutant allele fused to mCherry reporters respectively. As shown, #P<0, *P<0.05, **P<0.01, ***P<0.001.

FIGS. 20A-D show information relating to M7.8L shRNA allele specific silenced MYL2-47K mutation in Neonatal human double transgenic cardiomyocytes. FIG. 20A shows mRNA level quantification of the human normal and mutated alleles using quantitative PCR and specific blockers 4d after transduction with AAV9 expressing M7.8L shRNA and Cerulean reporter. FIG. 20B shows single nucleotide quantification of the normal ‘C’ and variant ‘A’ using pyrosequencing 4d after transduction with AAV9 expressing M7.8L shRNA and Cerulean reporter. FIG. 20C shows contraction percentage of single neonatal cardiomyocytes subjected to micropatterning and transduced with AAV9 expressing M7.8L shRNA. FIG. 20D shows at left: Mouse MYL2-N47K transgenic neonatal cardiomyocytes transduced with AAV9 expressing M7.8L shRNA and cerulean reporter, middle: Mouse MYL2-N47K transgenic neonatal cardiomyocytes cultured in micropatterning wells, and at right: Neonatal cardiomyocyte in microppatterning wells.

FIGS. 21A-G show information relating to AAV9 M7.8L shRNA allele specific silenced MYL2-47K mutation in mutant transgenic mice during 4 months treatment.

FIGS. 21H-I show information relating to AAV9 M7.8L shRNA allele specific silencing of MYL2-47K mutation in vivo of human mutant transgenic mouse hearts with trend toward improvement of ejection fraction (FIG. 21H) and significant reduction of left ventricular mass (FIG. 21J) (p=0.02) by echocardiography during 4 months of treatment.

FIG. 21J-K. show information relating to AAV9 M7.8L shRNA allele specific silencing of MYL2-47K mutation in vivo of human double transgenic (mutant/wildtype) mouse hearts with trend toward improvement of ejection fraction (FIG. 21J) and significant reduction of left ventricular mass (FIG. 21K) (p<0.05) by echocardiography during 4 months of treatment.

FIG. 22 shows information relating to M7.8L shRNA silenced MYL2-47K mutation in vivo and decreased the expression of hypertrophic biomarkers. Among other things, shown are mRNA levels of hypertrophic biomarkers and calcium regulators in MYL2 human mutant transgenic (mutTg) mice at 4 months of age and treated at 3 days old with M7.8L RNAi. UT=Untreated; Ctrl=mice treated with AAV9 non-expressing shRNA; M7.8L=mice treated with M7.8L RNAi. #P<0, *P<0.05, **P<0.01, ***P<0.001.

FIGS. 23A-B shows information relating to H10.8L and H11.8L shRNA silenced MYHY-403Q mutation. As shown, UT=Untreated; Ctrl=mice treated with AAV9 non-expressing shRNA; M7.8L=mice treated with M7.8L RNAi. #P<0, *P<0.05, **P<0.01, ***P<0.001.

FIG. 24 show results that indicate fold change in wild type (WT) and mutant (MUT) MYH7 alleles. As shown, AAV6-shRNA-transduced-cell expression of each MYH7 allele is normalized to control expression. WT p-value=0.0408. MUT p-value=0.0199. Both the wild type and the mutant allele are significantly decreased. Error bars are standard deviation between transduced wells.

FIG. 25 show results that indicate fold change in wild type (WT) and mutant (MUT) MYH7 alleles. AAV6-shRNA-transduced-cell expression of each MYH7 allele is normalized to control expression. Samples with an 18S Ct value above 17 for the wild type allele QPCR reaction were removed. Samples with any “Undetermined” Ct values were also removed. WT p-value=0.1207. MUT p-value<0.0001. Error bars are standard deviation between transduced wells. The mutant allele is significantly reduced while wild type allele is not. Trial two shows the potential of allele-specific shRNAs delivered by AAV vectors to specifically silence a mutant allele.

FIGS. 26A-E depict a flowchart of identifying candidate shRNAs according to an embodiment of the present invention.

FIG. 27 is a flowchart of identifying candidate shRNAs according to an embodiment of the present invention.

FIG. 28 is a block diagram of a computer system on which certain methods of the present invention may be implemented.

FIG. 29 is a table showing certain results from testing performed according to certain embodiments of the present invention.

### DETAILED DESCRIPTION OF THE INVENTION

The practice of embodiments of the present invention will employ, unless otherwise indicated, conventional methods of medicine, chemistry, biochemistry, molecular biology and recombinant DNA techniques, within the skill of the art. Such techniques are explained fully in the literature. See, e.g., siRNA Design: Methods and Protocols (Methods in Molecular Biology, D. J. Taxman ed., Humana Press, 2013); siRNA and miRNA Gene Silencing: From Bench to Bedside (Methods in Molecular Biology, M. Sioud ed., Humana Press, 2009); RNA Interference (Current Topics in Microbiology and Immunology, P. J. Paddison and P. K. Vogt eds., Springer, 1st edition, 2008); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.). All publications, patents and patent applications cited herein, whether supra or infra, are hereby incorporated by reference in their entireties.

In describing the present invention, the following terms may be employed, and are intended to be defined as indicated below.

It must be noted that, as used in this specification and the appended claims, the singular forms “a”, “an” and “the” include plural referents unless the content clearly dictates otherwise. Thus, for example, reference to “an RNA” includes a mixture of two or more RNAs, and the like.

The term “RNA interference oligonucleotide” or “RNAi oligonucleotide” refers to RNA and RNA-like molecules that can interact with the RNA-induced silencing complex (RISC) to guide downregulation of target transcripts based on sequence complementarity to the RNAi oligonucleotide. One strand of the RNAi oligonucleotide is incorporated into RISC, which uses this strand to identify mRNA molecules that are at least partially complementary to the incorporated RNAi oligonucleotide strand, and then cleaves these target mRNAs or inhibits their translation. The RNAi oligonucleotide strand that is incorporated into RISC is known as the guide strand and is usually the antisense strand. RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein by translational repression without cleavage of the target mRNA. Examples of RNA molecules that can interact with RISC include small interfering RNAs (siRNAs), short hairpin RNAs (shRNAs), microRNAs (miRNAs), and dicer-substrate 27-mer duplexes. The term includes RNA molecules containing one or more chemically modified nucleotides, one or more deoxyribonucleotides, and/or one or more non-phosphodiester linkages or any other RNA or RNA-like molecules that can interact with RISC and participate in RISC-mediated changes in gene expression.

As used herein, the term “small interfering RNA” or “siRNA” refers to double-stranded RNA molecules, comprising a sense strand and an antisense strand, having sufficient complementarity to one another to form a duplex. Such sense and antisense strands each have a region of complementarity ranging, for example, from about 10 to about 30 contiguous nucleotides that base pair sufficiently to form a duplex or double-stranded siRNA according to certain embodiments of the present invention. Such siRNAs are able to specifically interfere with the expression of a gene by triggering the RNAi machinery (e.g., RISC) of a cell to remove RNA transcripts having identical or homologous sequences to the siRNA sequence. As described herein, the sense and antisense strands of an siRNA may each consist of only complementary regions, or one or both strands may comprise additional sequences, including non-complementary sequences, such as 5′ or 3′ overhangs. In certain embodiments, an overhang may be of any length of nonhomologous residues, for example, 2, 3, 4, 5, 6, 7, 8, 9, 10, 11, 12, 13, 14, 15, 16 or more nucleotides. In addition, siRNAs may have other modifications, such as, for example, substituted or modified nucleotides or other sequences, which contribute to either the stability of the siRNA, its delivery to a cell or tissue, or its potency in triggering RNAi. It is to be understood that the terms “strand” and “oligonucleotide” may be used interchangeably in reference to the sense and antisense strands of siRNA compositions.

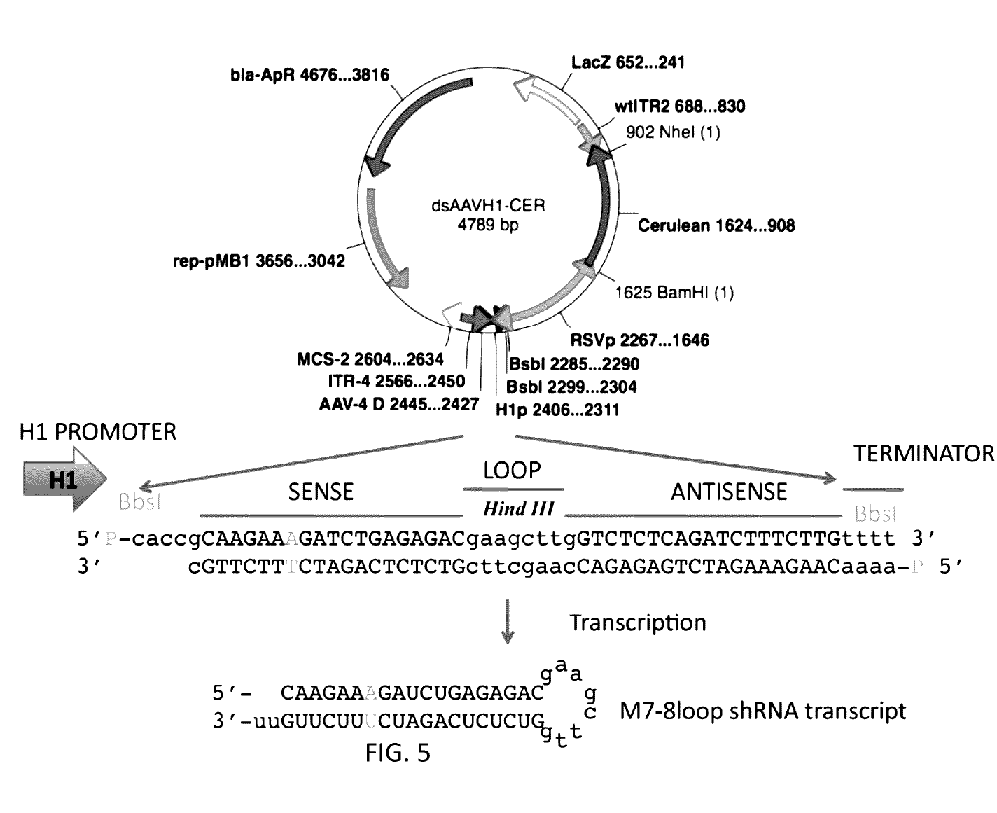

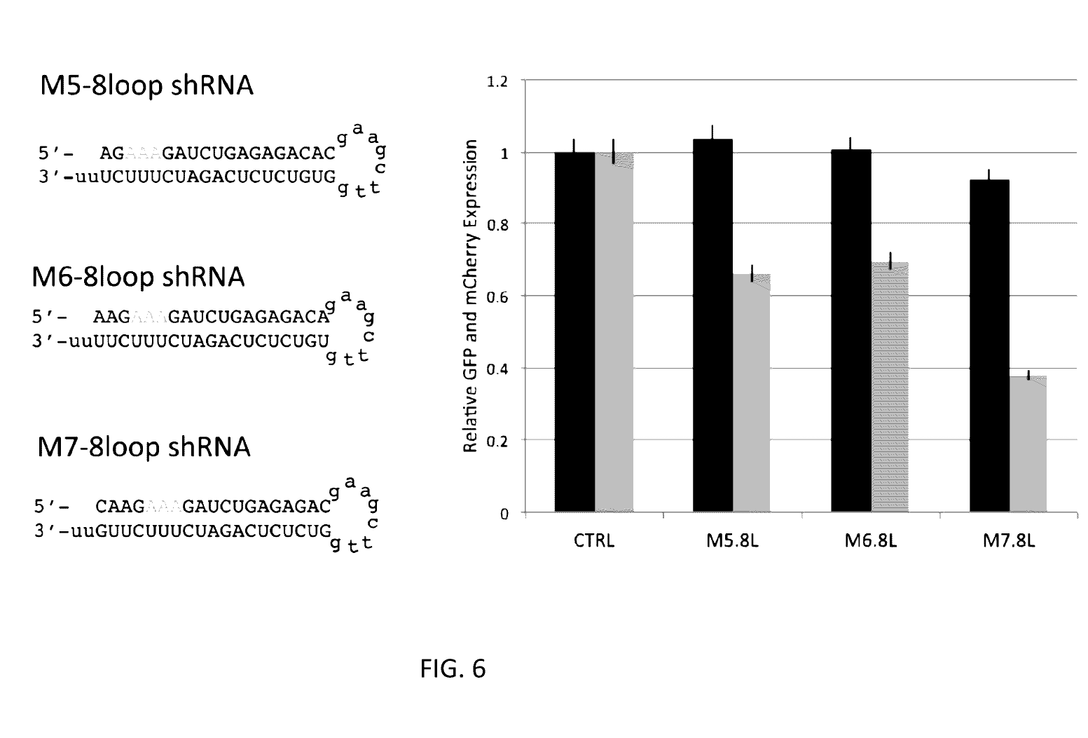

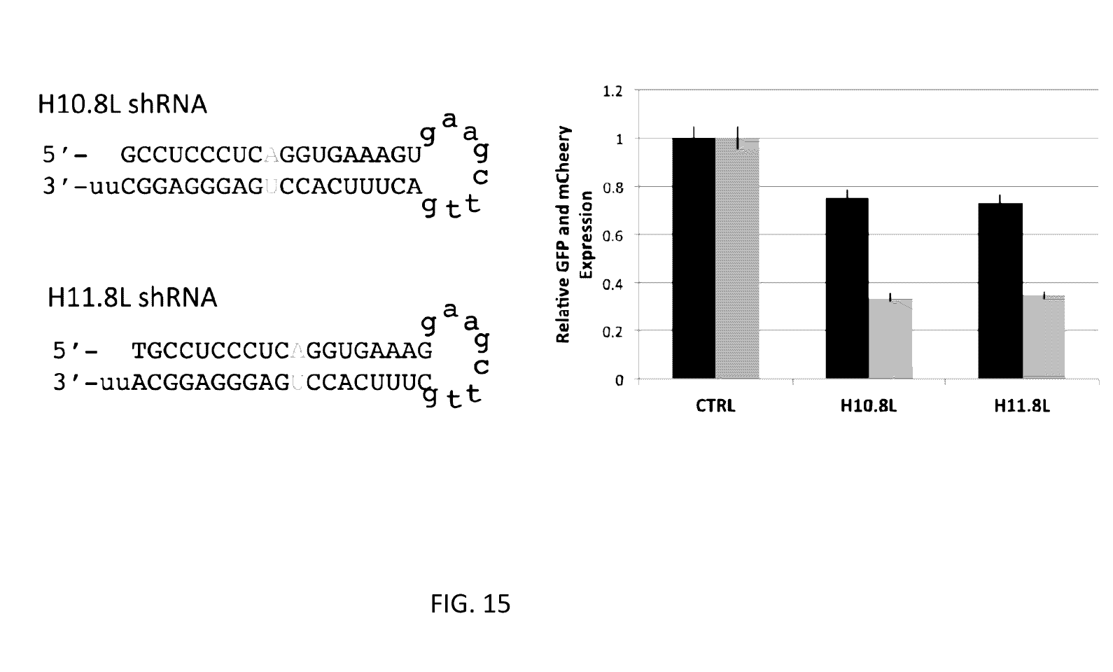

As used herein, the term “small hairpin RNA” or “shRNA” refers to an RNA sequence comprising a double-stranded stem region and a loop region at one end forming a so-called hairpin loop. In certain embodiments, the double-stranded region is typically about 19 nucleotides to about 30 nucleotides in length on each side of the stem, and the loop region is typically about three to about twelve nucleotides in length. In certain embodiments, the shRNA may include 3′- or 5′-terminal single-stranded overhangs. An overhang may be of any length of nonhomologous residues, for example, 2, 3, 4, 5, 6, 7, 8, 9, 10, 11, 12, 13, 14, 15, 16, or more nucleotides. In addition, such shRNAs may have other modifications, such as, for example, substituted or modified nucleotides or other sequences, which contribute to either the stability of the shRNA, its delivery to a cell or tissue, or its potency in triggering RNAi. In some cases, the shRNA may be derived from an siRNA, the shRNA comprising the sense strand and antisense strand of the siRNA connected by a loop (see, e.g., FIGS. 5, 6, and 15 showing exemplary shRNAs). For example, FIG. 5 shows the design and cloning of shRNAs in the AAV9 vector pAAV-H1p RSVp-Cerulean. FIG. 6 shows fluorescence activated cell sorting (FACS) of stable transfected HEK cells with MYL2-47N-GFP and MYL2-47K-mCherry and transfected with plasmid expressing shRNAs: M5.8L, M6.8L and M7.8L. And, FIG. 15 shows fluorescence activated cell sorting of the relative GFP and mCherry expression of double stable transfected human embryonic kidney cells containing MYH7-403R-GFP and MYH7-403Q-mCherry and transfected with H10.8L and H11.8L shRNAs. Further details regarding these figures will be described below.

The terms “hybridize” and “hybridization” refer to the formation of complexes between nucleotide sequences which are sufficiently complementary to form complexes via Watson-Crick base pairing.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. When a uracil is denoted in the context of the present invention, however, the ability to substitute a thymine is implied, unless otherwise stated. “Complementarity” may exist between two RNA strands, two DNA strands, or between a RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art. For purposes of RNAi, sense and antisense strands of an siRNA or sense and antisense sequences of a shRNA composition may be deemed “complementary” if they have sufficient base-pairing to form a duplex (i.e., they hybridize with each other at a physiological temperature). The antisense (guide) strand of an siRNA or shRNA directs RNA-induced silencing complex (RISC) to mRNA that has a complementary sequence.

A “target site” is the nucleic acid sequence recognized by an RNAi oligonucleotide (e.g., siRNA or shRNA). Typically, the target site is located within the coding region of a mRNA. The target site may be allele-specific (e.g., human myosin MYH7 allele encoding MHC-403Q or human MYL2 allele encoding RLC-47K).

“Administering” an RNAi oligonucleotide (e.g., siRNA or shRNA) or an expression vector or nucleic acid encoding an RNAi oligonucleotide to a cell comprises transducing, transfecting, electroporating, translocating, fusing, phagocytosing, shooting or ballistic methods, etc., e.g., any means by which a nucleic acid can be transported across a cell membrane.

The term “downregulating expression” refers to reduced expression of an mRNA or protein after administering or expressing an amount of an RNAi oligonucleotide (e.g., an siRNA or shRNA). An RNAi oligonucleotide may downregulate expression, for example, by reducing translation of the target mRNA into protein, for example, through mRNA cleavage or through direct inhibition of translation. The reduction in expression of the target mRNA or the corresponding protein is commonly referred to as “knockdown.” Downregulation or knockdown of expression may be complete or partial (e.g., all expression, some expression, or most expression of the target mRNA or protein is blocked by an RNAi oligonucleotide). For example, an RNAi oligonucleotide may reduce the expression of a mRNA or protein by 25%-100%, 30%-90%, 40%-80%, 50%-75%, or any amount in between these ranges, including at least 25%, 30%, 40%, 50%, 60%, 70%, 80%, or 90%, as compared to native or control levels. Downregulation of a target mRNA or protein may be the result of administering a single RNAi oligonucleotide or multiple (i.e., two or more) RNAi oligonucleotides or vectors encoding them. According to embodiments of the present invention, downregulating can be achieved to 0%. Indeed, certain experiments have demonstrated downregulation of an individual sample to about 2%.

By “selectively binds” is meant that the molecule binds preferentially to the target of interest or binds with greater affinity to the target than to other molecules. For example, an RNAi oligonucleotide (e.g., siRNA or shRNA) will bind to a substantially complementary sequence and not to unrelated sequences. An oligonucleotide that “selectively binds” to a particular allele, such as a particular mutant human MYH7 or human MYL2 allele (e.g., MYH7 allele encoding MHC-403Q or MYL2 allele encoding RLC-47K), denotes an RNAi oligonucleotide (e.g., an siRNA or shRNA) that binds preferentially to the particular target allele, but to a lesser extent to a wild-type allele or other sequences. An RNAi oligonucleotide that selectively binds to a particular target mRNA will selectively downregulate expression of that target mRNA, that is, the expression of the target mRNA will be reduced to a greater extent than other mRNAs.

The term “derived from” is used herein to identify the original source of a molecule but is not meant to limit the method by which the molecule is made which can be, for example, by chemical synthesis or recombinant means.

By “isolated” when referring to a polynucleotide, such as a mRNA, RNAi oligonucleotide (e.g., siRNA or shRNA), or other nucleic acid is meant that the indicated molecule is present in the substantial absence of other biological macromolecules of the same type. Thus, an isolated siRNA or shRNA molecule refers to a polynucleotide molecule, which is substantially free of other polynucleotide molecules, e.g., other siRNA or shRNA molecules that do not target the same RNA nucleotide sequence. The molecule may, however, include some additional bases or moieties which do not deleteriously affect the basic characteristics of the composition.

“Substantially purified” generally refers to isolation of a substance (e.g., compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The terms “polynucleotide,” “oligonucleotide,” “nucleic acid,” and “nucleic acid molecule” are used herein to include a polymeric form of nucleotides of any length, either ribonucleotides or deoxyribonucleotides. These terms refer to the primary structure of the molecule. Thus, the terms include triple-, double- and single-stranded DNA, as well as triple-, double- and single-stranded RNA. Also included are modifications, such as by methylation and/or by capping, and unmodified forms of the polynucleotide. More particularly, the terms “polynucleotide,” “oligonucleotide,” “nucleic acid,” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid,” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, siRNA, shRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), cerulean fluorescent protein, Dronpa, mCherry, mOrange, mPlum, Venus, firefly luciferase, Renilla luciferase, NADPH, beta-galactosidase, horseradish peroxidase, glucose oxidase, alkaline phosphatase, chloramphenical acetyl transferase, urease, MRI contrast agents (e.g., gadodiamide, gadobenic acid, gadopentetic acid, gadoteridol, gadofosveset, gadoversetamide, and gadoxetic acid), and computed tomography (CT) contrast agents (e.g., Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, and Calcium iopodate).

“Recombinant” as used herein to describe a nucleic acid molecule means a polynucleotide of genomic, RNA, siRNA, shRNA, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below.

“Recombinant host cells,” “host cells,” “cells,” “cell lines,” “cell cultures,” and other such terms denoting microorganisms or higher eukaryotic cell lines cultured as unicellular entities, refer to cells which can be, or have been, used as recipients for recombinant vector or other transferred DNA or RNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of any one or more of transcription of a mRNA or RNAi oligonucleotide, such as an siRNA or shRNA, from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake of an RNAi oligonucleotide (e.g., siRNA or shRNA) or an expression vector comprising an RNAi oligonucleotide.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contain within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

The term “3′ overhang” refers to at least one unpaired nucleotide extending out from the 3′-end of at least one strand of a duplexed RNA (e.g., double-stranded siRNA or stem region of shRNA). Similarly, the term “5′ overhang” refers to at least one unpaired nucleotide extending out from the 5′-end of at least one strand of a duplexed RNA. An overhang may be of any length of nonhomologous residues, for example, 2, 3, 4, 5, 6, 7, 8, 9, 10, 11, 12, 13, 14, 15, 16 or more nucleotides.

The term “region” when applied to polynucleotides generally refers to a contiguous portion or sequence of a single-stranded or double-stranded polynucleotide molecule. However, the term “region” may also refer to an entire single-stranded or double-stranded polynucleotide molecule.

The term “physiological conditions” refers to conditions that approximate the chemical and/or temperature environment that may exist within the body of an individual, subject, or patient.

The term “physiological temperature” generally refers to a temperature present within the body of an individual, subject, or patient. The term “physiological temperature” may be assumed to be approximately 37° C. unless otherwise specified.

The term “sense RNA” refers to an RNA sequence corresponding to all or a portion of a coding sequence of a gene or all or a portion of a plus (+) strand or mRNA sequence generated from a gene, or an RNA sequence homologous thereto.

The term “antisense strand” refers to an RNA sequence corresponding to all or a portion of a template sequence of a gene, or a sequence homologous thereto, or a minus (−) strand or all or a portion of a sequence complementary to a mRNA sequence generated from a gene.

The term “hybridize” refers to associating two complementary nucleic acid strands to form a double-stranded molecule which may contain two DNA strands, two RNA strands, one DNA and one RNA strand, etc.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The term “about,” particularly in reference to a given quantity, is meant to encompass deviations of plus or minus five percent.

An “effective amount” of an RNAi oligonucleotide (e.g., siRNA or shRNA) or a recombinant polynucleotide or vector encoding an RNAi oligonucleotide is an amount sufficient to effect beneficial or desired results, such as an amount that downregulates expression of a target mRNA or protein (e.g., human myosin MYH7 allele encoding MHC-403Q or human MYL2 allele encoding RLC-47K). For an RNAi oligonucleotide (e.g., an siRNA or shRNA), an effective amount may reduce translation or increase degradation of the mRNA targeted by the RNAi oligonucleotide. An effective amount can be administered in one or more administrations, applications or dosages.

By “therapeutically effective dose or amount” of an RNAi oligonucleotide (e.g., siRNA or shRNA) or a recombinant polynucleotide or vector encoding an RNAi oligonucleotide is intended an amount that, when administered as described herein, brings about a positive therapeutic response, such as improved recovery from cardiomyopathy. Improved recovery may include a reduction in one or more cardiac symptoms, such as dyspnea, chest pain, heart palpitations, lightheadedness, or syncope. Additionally, a therapeutically effective dose or amount of an RNAi oligonucleotide or a recombinant polynucleotide or vector encoding an RNAi oligonucleotide may improve cardiomyocyte contractile strength and sarcomere alignment. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### Digital Computer System

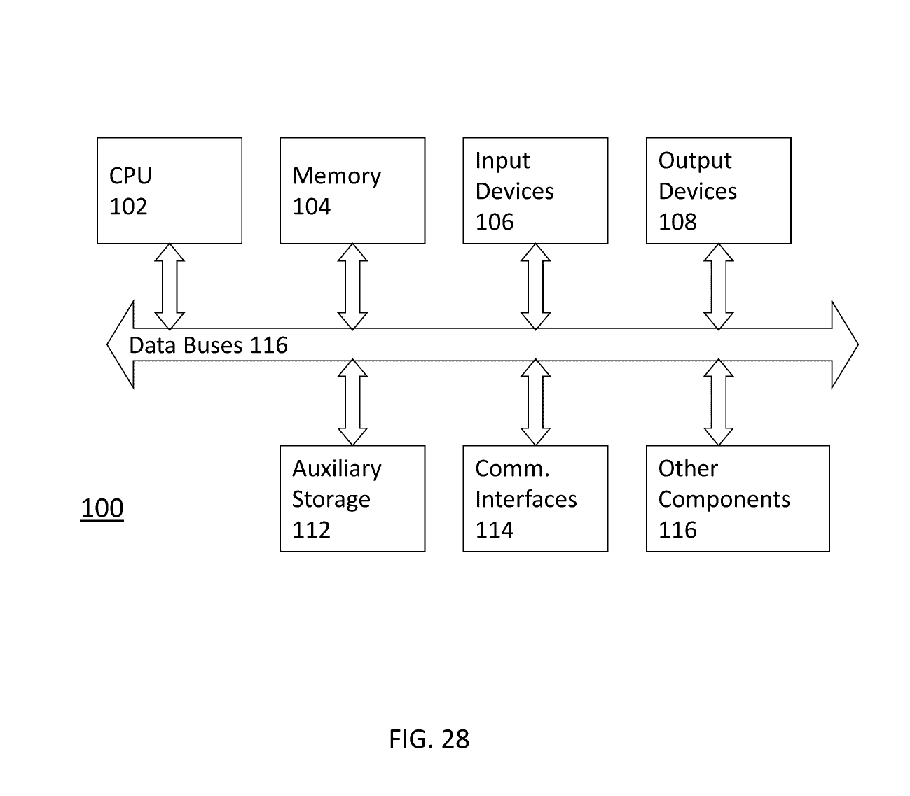

Among other things, the present invention relates to methods, techniques, and algorithms that are intended to be implemented in a digital computer system 100 such as generally shown in FIG. 28. Such a digital computer is well-known in the art and may include the following.

Computer system 100 may include at least one central processing unit 102 but may include many processors or processing cores. Computer system 100 may further include memory 104 in different forms such as RAM, ROM, hard disk, optical drives, and removable drives that may further include drive controllers and other hardware. Auxiliary storage 112 may also be include that can be similar to memory 104 but may be more remotely incorporated such as in a distributed computer system with distributed memory capabilities.

Computer system 100 may further include at least one output device 108 such as a display unit, video hardware, or other peripherals (e.g., printer). At least one input device 106 may also be included in computer system 100 that may include a pointing device (e.g., mouse), a text input device (e.g., keyboard), or touch screen.

Communications interfaces 114 also form an important aspect of computer system 100 especially where computer system 100 is deployed as a distributed computer system. Computer interfaces 114 may include LAN network adapters, WAN network adapters, wireless interfaces, Bluetooth interfaces, modems and other networking interfaces as currently available and as may be developed in the future.

Computer system 100 may further include other components 116 that may be generally available components as well as specially developed components for implementation of the present invention. Importantly, computer system 100 incorporates various data buses 116 that are intended to allow for communication of the various components of computer system 100. Data buses 116 include, for example, input/output buses and bus controllers.

Indeed, the present invention is not limited to computer system 100 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

The present disclosure provides a detailed explanation of the present invention with detailed explanations that allow one of ordinary skill in the art to implement the present invention into a computerized method. Certain of these and other details are not included in the present disclosure so as not to detract from the teachings presented herein but it is understood that one of ordinary skill in the art would be familiar with such details.

### MODES OF CARRYING OUT EMBODIMENTS OF THE INVENTION

It is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Cardiomyopathy is a genetic disease of the heart muscle caused by heterozygotic missense mutations in genes encoding proteins of the cardiac sarcomere. The present invention is based on the discovery of RNAi oligonucleotides, including siRNAs and shRNAs that selectively silence disease-causing alleles, including the myosin MYL2 allele encoding human regulatory light chain (hRLC)-N47K and the MYH7 allele encoding human myosin heavy chain (hMHC)-R403Q. The inventors have identified siRNAs and shRNAs that selectively downregulate these mutant alleles of MYL2 and MYH7 while retaining expression of the wild-type allele (see Example 1). Thus, the present invention pertains generally to compositions and methods for using RNAi oligonucleotides, including allele-selective silencing siRNAs and shRNAs for treatment of cardiomyopathy.

In one aspect, the invention provides a method for treating cardiomyopathy by utilizing RNAi oligonucleotides, including siRNAs and shRNAs that selectively target and downregulate expression of human myosin MYH7 and MYL2 mutant alleles associated with cardiomyopathy, including the MYH7 allele encoding MHC-R403Q and the MYL2 allele encoding RLC-N47K. The RNAi oligonucleotides of the invention selectively downregulate expression of these mutant alleles, for example, by reducing translation or increasing degradation of the target mRNA encoding the myosin heavy chain and regulatory light chain variant proteins. Preferably, one or more symptoms of cardiomyopathy are ameliorated or eliminated following administration of an RNAi oligonucleotide (e.g., siRNA or shRNA) resulting in improved cardiac function following treatment. Improved recovery may include, for example, a reduction in one or more cardiac symptoms, such as dyspnea, chest pain, heart palpitations, lightheadedness, or syncope. Additionally, treatment with an RNAi oligonucleotide may improve cardiomyocyte contractile strength and sarcomere alignment. Also, treatment with an RNAi oligonucleotide may improve functional capacity, heart structure, or heart function. Cardiomyopathies that can be treated by methods of the invention include, but are not limited to, dilated cardiomyopathy, hypertrophic cardiomyopathy, restrictive cardiomyopathy, arrhythmogenic right ventricular cardiomyopathy, and left ventricular noncompaction cardiomyopathy.

In certain embodiments, the RNAi oligonucleotide is an RNA or RNA-like molecule having a double stranded region that is at least partially identical and partially complementary to a target mRNA sequence, such as a mRNA sequence of a mutant human MYL2-N47K (SEQ ID NO:4) allele or human MYH7-R403Q (SEQ ID NO:42) allele. The RNAi oligonucleotide may be a double-stranded, small interfering RNA (siRNA) or a short hairpin RNA molecule (shRNA) comprising a stem-loop structure. The double-stranded regions of the RNAi oligonucleotide may comprise sequences that are at least partially identical and partially complementary, e.g., about 75%, 80%, 85%, 90%, 91%, 92%, 93%, 94%, 95%, 96%, 97%, 98%, or 99% identical and complementary, to the target mRNA sequence. In some embodiments, the double-stranded regions of the RNAi oligonucleotide comprise sequences that are at least substantially identical and substantially complementary to the target mRNA sequence. “Substantially identical and substantially complementary” refers to sequences that are at least about 95%, 96%, 97%, 98%, or 99% identical and complementary to a target polynucleotide sequence. In other embodiments, the double-stranded regions of the RNAi oligonucleotide may contain 100% identity and complementarity to the target mRNA sequence.

In certain embodiments, the RNAi oligonucleotide may comprise two complementary, single-stranded RNA molecules, such as an siRNA comprising sense and antisense strands. In other embodiments, the sense RNA sequence and the antisense RNA sequence may be encoded by a single molecule, such as an shRNA comprising two complementary sequences forming a “stem” (corresponding to sense and antisense strands) covalently linked by a single-stranded “hairpin” or loop sequence. The hairpin sequence may be from about 3 to about 12 nucleotides in length, including any length in between, such as 3, 4, 5, 6, 7, 8, 9, 10, 11, or 12 nucleotides in length. The loop can be at either end of the molecule; that is, the sense strand can be either 5′ or 3′ relative to the loop. In addition, a non-complementary duplex region (approximately one to six base pairs, for example, four CG base pairs) can be placed between the targeting duplex and the loop, for example to serve as a “CG clamp” to strengthen duplex formation. Exemplary hairpin sequences include a loop of 8 nucleotides in length comprising the sequence of CAAGCTTC or a loop of 12 nucleotides in length comprising the sequence of SEQ ID NO:1.

In certain embodiments, the sense RNA strand or sequence of the siRNA or shRNA is 19 to 29 nucleotides in length or any length in between, such as 19, 20, 21, 22, 23, 24, 25, 26, 27, 28, or 29 nucleotides in length. Similarly, the antisense strand or sequence of the siRNA or shRNA may be 19 to 29 nucleotides in length or any length in between, such as 19, 20, 21, 22, 23, 24, 25, 26, 27, 28, or 29 nucleotides in length. The regions of complementarity in sense and antisense strands or sequences may be the same length. Alternatively, the sense and antisense strands may further contain non-complementary sequences, such as 3′ or 5′ overhangs or other non-complementary sequences that provide different functions for the siRNA or shRNA composition that do not contribute to base-pairing between the sense and antisense strands or sequences. Overhangs may include ribonucleotides, deoxyribonucleotides, or chemically modified nucleotides that, for example, promote enhanced nuclease resistance.

In certain embodiments, an siRNA or shRNA may comprise a 3′ overhang of from 1 to about 6 nucleotides in length, such as an overhang of 1 to about 5 nucleotides in length, 1 to about 4 nucleotides in length, or 2 to 4 nucleotides in length, including any length within these ranges, such as 1, 2, 3, 4, or 5 nucleotides in length. Either one or both strands of an siRNA may comprise a 3′ overhang. If both strands of the siRNA comprise 3′ overhangs, the length of the overhangs may be the same or different for each strand. In one embodiment, the 3′ overhang present on either one or both strands of the siRNA may be 2 nucleotides in length. For example, each strand of an siRNA may comprise a 3′ overhang of dithymidylic acid (“TT”) or diuridylic acid (“UU”) or other effective dinucleotide combinations known in the art. The 3′ terminus of an shRNA can have a non-target-complementary overhang of two or more nucleotides, for example, UU or dTdT; however, the overhangs can comprise any nucleotide including chemically modified nucleotides that, for example, promote enhanced nuclease resistance. In other embodiments, siRNAs or shRNAs comprise one or zero nucleotides overhanging at the 3′ end.

In order to enhance stability of an siRNA or shRNA, 3′ overhangs may be stabilized by including purine nucleotides, such as adenosine or guanosine nucleotides. Alternatively, substitution of pyrimidine nucleotides by modified analogues, e.g., substitution of uridine nucleotides in 3′ overhangs with 2′-deoxythymidine, may be tolerated and not affect the efficiency of RNAi degradation. In particular, the absence of a 2′-hydroxyl in the 2′-deoxythymidine may significantly enhance the nuclease resistance of the 3′ overhang.

RNAi oligonucleotides may further comprise one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. Additionally, the RNAi oligonucleotide may be conjugated to a lipophilic molecule (e.g., cholesterol or fatty acid) to facilitate cellular uptake. Although predominantly composed of ribonucleotides, siRNAs or shRNAs may also contain one or more deoxyribonucleotides in addition to ribonucleotides along the length of one or both strands or sequences to improve efficacy or stability. The 5′ end of one or both strands or sequences of an siRNA or shRNA may also contain a phosphate group.

In certain embodiments the invention includes an RNAi oligonucleotide that selectively downregulates expression of a regulatory light chain variant comprising a lysine substitution at position 47 (RLC-47K), wherein the RNAi oligonucleotide comprises: a) a sense strand comprising a sequence selected from the group consisting of SEQ ID NOS:10-12 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the RNAi oligonucleotide reduces expression of the RLC-47K; and b) an antisense strand comprising a region that is complementary to the sense strand. In one embodiment, the RNAi oligonucleotide is an siRNA that selectively downregulates expression of RLC-47K selected from the group consisting of: a) an siRNA comprising a sense strand comprising the sequence of SEQ ID NO:10 and an antisense strand comprising the sequence of SEQ ID NO:25; b) an siRNA comprising a sense strand comprising the sequence of SEQ ID NO:26 and an antisense strand comprising the sequence of SEQ ID NO:27; c) an siRNA comprising a sense strand comprising the sequence of SEQ ID NO:31 and an antisense strand comprising the sequence of SEQ ID NO:32; d) an siRNA comprising a sense strand comprising the sequence of SEQ ID NO:10 and an antisense strand comprising the sequence of SEQ ID NO:27; e) an siRNA comprising a sense strand comprising the sequence of SEQ ID NO:11 and an antisense strand comprising the sequence of SEQ ID NO:92; f) an siRNA comprising a sense strand comprising the sequence of SEQ ID NO:12 and an antisense strand comprising the sequence of SEQ ID NO:93; g) an siRNA comprising a sense strand comprising the sequence of SEQ ID NO:106 and an antisense strand comprising the sequence of SEQ ID NO:107; h) an siRNA comprising a sense strand comprising the sequence of SEQ ID NO:108 and an antisense strand comprising the sequence of SEQ ID NO:109; i) an siRNA comprising a sense strand comprising the sequence of SEQ ID NO:110 and an antisense strand comprising the sequence of SEQ ID NO:111; j) an siRNA comprising a sense strand comprising the sequence of SEQ ID NO:112 and an antisense strand comprising the sequence of SEQ ID NO:113; k) an siRNA comprising a sense strand comprising the sequence of SEQ ID NO:114 and an antisense strand comprising the sequence of SEQ ID NO:115; 1) an siRNA comprising a sense strand comprising the sequence of SEQ ID NO:116 and an antisense strand comprising the sequence of SEQ ID NO:117; and m) an siRNA comprising a sense strand comprising the sequence of SEQ ID NO:118 and an antisense strand comprising the sequence of SEQ ID NO:119. In another embodiment, the RNAi oligonucleotide is an shRNA that selectively downregulates expression of RLC-47K comprising a sequence selected from the group consisting of SEQ ID NOS:35-37.

In other embodiments, the invention includes an RNAi oligonucleotide that selectively downregulates expression of a human myosin heavy chain variant comprising a glutamine substitution at position 403 (MHC-403Q), wherein the RNAi oligonucleotide comprises: a) a sense strand comprising a sequence selected from the group consisting of SEQ ID NO:53 and SEQ ID NO:54 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the RNAi oligonucleotide reduces expression of the MHC-403Q; and b) an antisense strand comprising a region that is complementary to the sense strand. In one embodiment, the RNAi oligonucleotide is an shRNA that selectively downregulates expression of MHC-403Q comprising a sequence selected from the group consisting of SEQ ID NO:64 and SEQ ID NO:65.

In certain embodiments, the invention includes compositions comprising one or more RNAi oligonucleotides (e.g. siRNAs or shRNAs). Such compositions may comprise partially purified RNA, substantially pure RNA, synthetic RNA, or recombinantly produced RNA, as well as altered RNA that differs from naturally-occurring RNA by the addition, deletion, substitution, synthesis, and/or modification of one or more nucleotides. Such modifications may include addition of non-nucleotide material, such as to the end(s) of the siRNA or to one or more internal nucleotides of the siRNA, including modifications that make the siRNA or shRNA more effective or resistant to nuclease digestion.

Knockdown can be assessed by measuring levels of the mRNA targeted by RNAi oligonucleotides using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knockdown include RNA solution hybridization, nuclease protection, northern hybridization, gene expression monitoring with a microarray, antibody binding, radioimmunoassay, and fluorescence activated cell analysis.

In certain embodiments, a subject undergoing treatment for cardiomyopathy may first be genotyped to determine which mutant disease-causing allele is present in the subject to allow an appropriate treatment targeting the specific disease-causing allele. In one embodiment, the subject undergoing treatment has been shown by genotyping to have the MYH7 allele encoding human myosin heavy chain (MHC)-403Q and is administered a composition comprising one or more RNAi oligonucleotides or recombinant polynucleotides encoding one or more RNAi oligonucleotides that selectively downregulate expression of MHC-403Q. In another embodiment, the subject undergoing treatment has been shown by genotyping to have the MYL2 allele encoding regulatory light chain (RLC)-47K and is administered a composition comprises one or more RNAi oligonucleotides or recombinant polynucleotides encoding one or more RNAi oligonucleotides that selectively downregulate expression of RLC-47K, said RNAi oligonucleotides.

In another embodiment, the invention includes a method of downregulating expression of RLC-47K or MHC-403Q in a subject, the method comprising: administering an effective amount of an RNAi oligonucleotide (e.g., siRNA or an shRNA) described herein to the subject.

In another embodiment, the invention includes a method of downregulating expression of RLC-47K or MHC-403Q in a cardiac cell (e.g. cardiomyocyte), the method comprising introducing an effective amount of an RNAi oligonucleotide (e.g., siRNA or an shRNA) described herein into the cell.

In another aspect, the invention includes a method for selectively decreasing the amount of a RLC-47K or MHC-403Q protein in a cardiac cell of a subject, the method comprising introducing an effective amount of an RNAi oligonucleotide (e.g., siRNA or an shRNA) described herein into the cardiac cell of the subject.

In certain embodiments, the RNAi oligonucleotide (e.g., siRNA or shRNA) is expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, an expression vector comprises a promoter “operably linked” to at least one polynucleotide encoding an RNAi oligonucleotide (e.g., siRNA or shRNA). The phrase “operably linked” or “under transcriptional control” as used herein means that the promoter is in the correct location and orientation in relation to a polynucleotide to control the initiation of transcription by RNA polymerase and expression of the polynucleotide. In one embodiment, the recombinant polynucleotide comprises a first polynucleotide sequence encoding the sense strand of an siRNA and a second polynucleotide sequence encoding the antisense strand of an siRNA. In another embodiment, the recombinant polynucleotide comprises a polynucleotide sequence encoding an shRNA, including the sense sequence, antisense sequence, and hairpin loop of the shRNA. Exemplary sequences of constructs comprising an expression vector encoding an shRNA are shown in SEQ ID NO:120, SEQ ID NO:124, and SEQ ID NO:125.

In certain embodiments, the nucleic acid encoding a polynucleotide of interest is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) (1985) 41:521, such as elements included in the CMV intron A sequence.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458).

Additionally, 5′-UTR sequences can be placed adjacent to the coding sequence in order to enhance expression of the same. Such sequences include UTRs which include an Internal Ribosome Entry Site (IRES) present in the leader sequences of picornaviruses such as the encephalomyocarditis virus (EMCV) UTR (Jang et al. J. Virol. (1989) 63:1651-1660. Other picornavirus UTR sequences that will also find use in the present invention include the polio leader sequence and hepatitis A virus leader and the hepatitis C IRES.

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Fluorescent markers (e.g., GFP, EGFP, Dronpa, mCherry, mOrange, mPlum, Venus, YPet, phycoerythrin), or immunologic markers can also be employed. The selectable marker employed is not believed to be important, so long as it is capable of being expressed simultaneously with the nucleic acid encoding a gene product. Further examples of selectable markers are well known to one of skill in the art.

There are a number of ways in which expression vectors may be introduced into cells. In certain embodiments of the invention, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

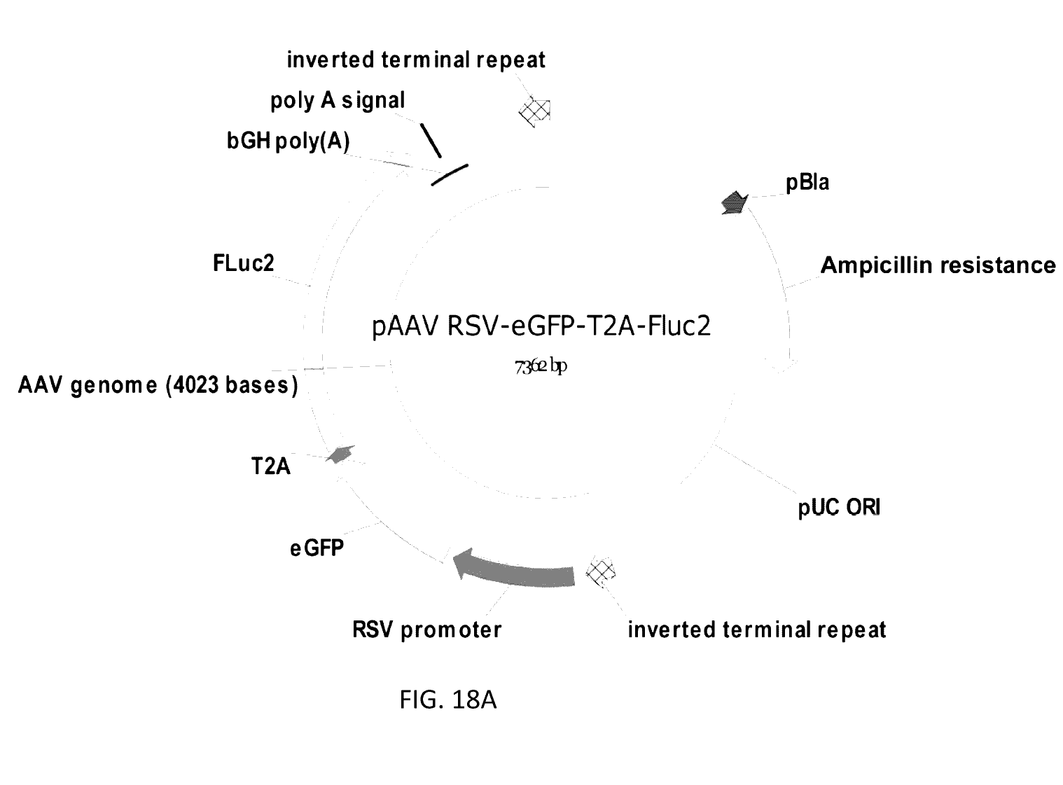

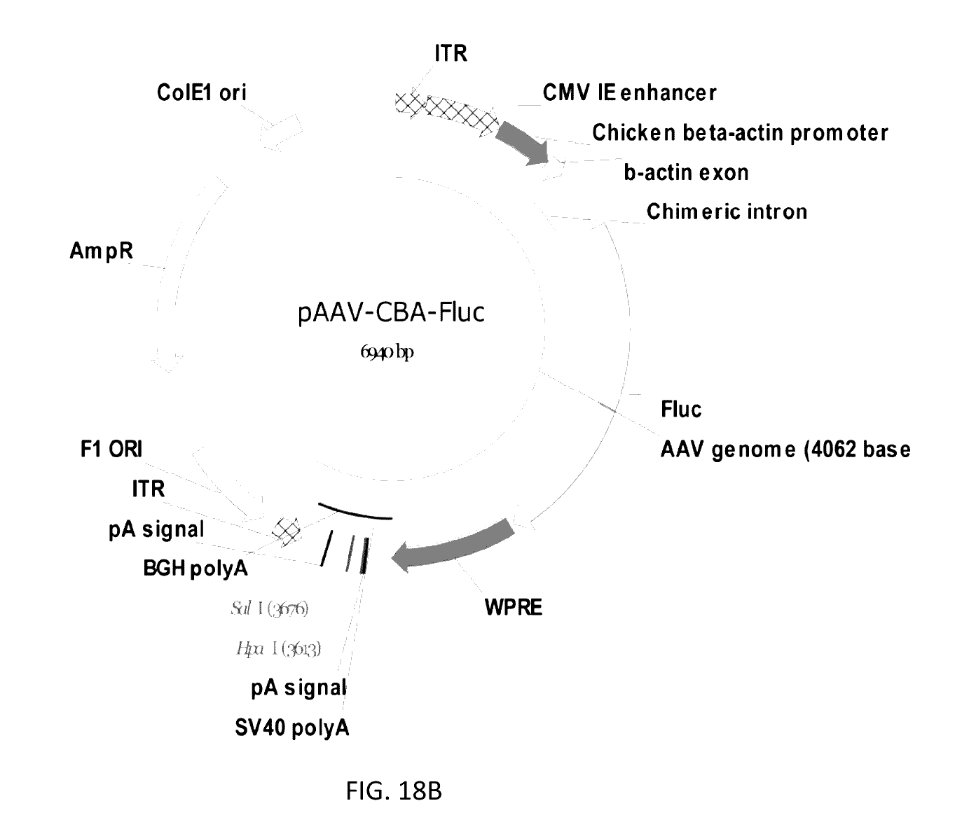

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875. Exemplary AAV vectors are presented in FIGS. 5, 18A, and 18B and the Sequence Listing (SEQ ID NOS:121-123), and exemplary constructs comprising expression vectors encoding shRNAs (SEQ ID NO:120, SEQ ID NO:124, and SEQ ID NO:125) and their use in expressing RNAi oligonucleotides are described in Example 1.

For example, FIG. 5 shows the design and cloning of shRNAs in the AAV9 vector pAAV-H1p RSV_{P}-Cerulean. Also, FIGS. 18A and 18B show AAV9-Luciferase viral vectors expressing M7.8L shRNA under an H1 promoter for in vivo experiments in mice containing human MYL2 wild type and mutant transgenes. FIG. 18A shows a schematic of the pAAV-RSV-eGFP-T2A-Fluc2 vector (SEQ ID NO:123). FIG. 18B shows a schematic of the pAAV-CBA-Fluc vector (SEQ ID NO:122).

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., encoding siRNA or shRNA) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545. Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia based infection/transfection system can be conveniently used to provide for inducible, transient expression of the polynucleotides of interest (e.g., encoding siRNA or shRNA) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau and Sene (1982) Biochim. Biophys. Acta 721:185-190; Fraley et al. (1979) Proc. Natl. Acad. Sci. USA

76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid encoding the RNAi oligonucleotide may be positioned and expressed at different sites. In certain embodiments, the nucleic acid encoding the RNAi oligonucleotide may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty and Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding a RNAi oligonucleotide may also be transferred in a similar manner in vivo and express the RNAi oligonucleotide.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat (1991) Liver Diseases, Targeted Diagnosis and Therapy Using Specific Receptors and Ligands, Wu et al. (Eds.), Marcel Dekker, N.Y., 87-104). Also contemplated is the use of lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular RNAi oligonucleotide into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). Recently, a synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, an oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

The RNAi oligonucleotide (e.g., siRNA or shRNA) may comprise a detectable label in order to facilitate detection of binding of the RNAi oligonucleotide to a target nucleic acid. Detectable labels suitable for use in the present invention include any composition detectable by spectroscopic, photochemical, biochemical, immunochemical, electrical, optical, or chemical means. Useful labels in the present invention include biotin or other streptavidin-binding proteins for staining with labeled streptavidin conjugate, magnetic beads (e.g., Dynabeads), fluorescent dyes (e.g., green fluorescent protein, mCherry, cerulean fluorescent protein, phycoerythrin, YPet, fluorescein, texas red, rhodamine, and the like, see, e.g., Molecular Probes, Eugene, Oreg., USA), radiolabels (e.g., 3H, 125I, 35S, 14C, or 32P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. In addition, magnetic resonance imaging (MRI) contrast agents (e.g., gadodiamide, gadobenic acid, gadopentetic acid, gadoteridol, gadofosveset, gadoversetamide, gadoxetic acid), and computed tomography (CT) contrast agents (e.g., Diatrizoic acid, Metrizoic acid, Iodamide, lotalamic acid, loxitalamic acid, loglicic acid, Acetrizoic acid, locarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, Calcium iopodate) are useful as labels in medical imaging. Patents teaching the use of such labels include U.S. Pat. Nos. 3,817,837; 3,850,752; 3,939,350; 3,996,345; 4,277,437; 4,275,149; 4,366,241; 5,798,092; 5,695,739; 5,733,528; and 5,888,576.

The present invention also encompasses pharmaceutical compositions comprising one or more of RNAi oligonucleotides (e.g., siRNAs or shRNAs) or recombinant polynucleotides or vectors encoding them and a pharmaceutically acceptable carrier. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, may be used as delivery vehicles for the of RNAi oligonucleotides (e.g., siRNAs or shRNAs) or recombinant polynucleotides or vectors encoding them described herein. Commercially available fat emulsions that are suitable for delivering the nucleic acids of the invention to tissues, such as cardiac muscle tissue and smooth muscle tissue, include Intralipid, Liposyn, Liposyn II, Liposyn III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449, which are herein incorporated by reference in their entireties.

One will generally desire to employ appropriate salts and buffers to render delivery vehicles stable and allow for uptake by target cells. Buffers also will be employed when recombinant cells are introduced into a patient. Aqueous compositions of the present invention comprise an effective amount of the delivery vehicle, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the nucleic acids of the compositions.

Compositions for use in the invention will comprise a therapeutically effective amount of at least one RNAi oligonucleotide (e.g., siRNA or shRNA) or recombinant polynucleotide or vector encoding an RNAi oligonucleotide. By “therapeutically effective dose or amount” of an RNAi oligonucleotide or recombinant polynucleotide or vector encoding an RNAi oligonucleotide is intended an amount that, when administered as described herein, brings about a positive therapeutic response, such as improved recovery from cardiomyopathy. Improved recovery may include a reduction in one or more cardiac symptoms, such as dyspnea, chest pain, heart palpitations, lightheadedness, or syncope. Additionally, a therapeutically effective dose or amount of an RNAi oligonucleotide or recombinant polynucleotide or vector encoding an RNAi oligonucleotide may improve cardiomyocyte contractile strength and sarcomere alignment.

An “effective amount” of an RNAi oligonucleotide (e.g., siRNA or shRNA) or a recombinant polynucleotide or vector encoding an RNAi oligonucleotide is an amount sufficient to effect beneficial or desired results, such as an amount that downregulates expression of a target mRNA or protein (e.g., human myosin MYH7 allele encoding MHC-403Q or human MYL2 allele encoding RLC-47K). For an RNAi oligonucleotide (e.g., an siRNA or shRNA), an effective amount may reduce translation or increase degradation of the mRNA targeted by the RNAi oligonucleotide. An effective amount can be administered in one or more administrations, applications or dosages. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

Once formulated, the compositions are conventionally administered parenterally, e.g., by injection intracardially, intramyocardially, intraventricularly subcutaneously, intraperitoneally, intramuscularly, intra-arterially, or intravenously. In one embodiment, compositions are administered locally by injection into the heart. Compositions may be injected directly into cardiomyocytes. Additional formulations suitable for other modes of administration include oral and pulmonary formulations, suppositories, and transdermal formulations, aerosol, intranasal, and sustained release formulations.

Dosage treatment may be a single dose schedule or a multiple dose schedule. The exact amount necessary will vary depending on the desired response; the subject being treated; the age and general condition of the individual to be treated; the severity of the condition being treated; the mode of administration, among other factors. An appropriate effective amount can be readily determined by one of skill in the art. A “therapeutically effective amount” will fall in a relatively broad range that can be determined through routine trials using in vitro and in vivo models known in the art.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Any of the compositions described herein may be included in a kit. For example, at least one RNAi oligonucleotide (e.g., siRNA or shRNA) or recombinant polynucleotide or vector encoding and RNAi oligonucleotide, may be included in a kit. The kit may also include one or more transfection reagents to facilitate delivery of polynucleotides to cells.

The components of the kit may be packaged either in aqueous media or in lyophilized form. The container means of the kits will generally include at least one vial, test tube, flask, bottle, syringe or other container means, into which a component may b placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the RNAi oligonucleotides/nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain the RNAi oligonucleotides/nucleic acids or that protect against their degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented. A kit may also include utensils or devices for administering the RNAi oligonucleotide (e.g., siRNA or shRNA) or recombinant polynucleotide or vector encoding an RNAi oligonucleotide by various administration routes, such as parenteral or catheter administration or coated stent.

### EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should be considered.

### EXEMPLIFICATION

### Example 1

### Oligonucleotide Therapeutic Approaches for Allele Silencing of HRLC-47K and HMHC-403Q Mutations in Hypertrophic Cardiomyopathy

We hypothesized that the delivery of oligonucleotides reagents with allele-specific silencing capabilities might abrogate the negative effects of the disease hypertrophic cardiomyopathy. We focused on silencing the alleles of two mutations: R403Q and N47K of the β-MHC and RLC, respectively. The R403Q mutation alters the binding between the myosin head domain with actin causing a lack of Z-line sarcomeric alignment and as consequence variations in myocyte shape and contractile failure. The R403Q allele is a “gain of function” mutation based on single molecule studies showing increased generation of force and faster actin filament sliding. On the other hand, the RLC N47K mutation affects the rotation of the lever arm. This rotation is important to create a large angle to replace the myosin head in a different position on the actin protein domain and generate the “power stroke” playing a key role in contraction. The mutation is near to the calcium-binding site causing a reduction of the mechanical force. The mutation causes “loss of function” and results in compensatory hypertrophy.

Certain aspects of oligonucleotide silencing of R403Q and N47K mutations using human iPSc-CM cell model or mouse NCM model are still in a preliminary stage due to the lack of cardiac maturity, where the β-MHC isoform is prevalent. Recently findings provide direct evidence that miRNAs are involved in cardiac development but also in heart failure. However, an effort to mature these cells has been partially achieved with a high-throughput technique to measure single cell function using micro-patterning polyacrylamide devices and successfully induced sarcomeric alignment and nuclear morphologies similar to those observed in adult cardiomyocytes.

Research regarding oligonucleotide (siRNA) delivery in primary cells and in the whole heart is ongoing. Heart cells are difficult to transfect, but viral delivery of short hairpin RNAs (shRNAs) with adeno-associate virus (AAV) show promise for gene transfer. The non-pathogenic nature of AAV has not been associated with any disease in humans, making them potentially powerful gene therapy vehicles.

### Materials and Methods

The Stanford University Human Research Institutional Review Board approved all the protocols for iPS cell reprogramming and cardiac differentiation study. The administrative panel on laboratory animal care (APLAC) from Stanford University approved all the protocols for mice neonatal heart isolation and adeno-associated virus (AAV) injections. All oligonucleotide primers for PCR and quantitative PCR, siRNAs and shRNAs were synthesized by the protein and nucleic acid (PAN) facility from Stanford University. Plasmid preparation, DNA and RNA extractions were carried out with Qiagen kits (Valencia, Calif.) and all other DNA manipulations were carried out according to standard methods. Escherichia coli strain DH5□∇ was used as the host for general plasmid DNA propagation.

Small Interfering RNA (siRNA) Design

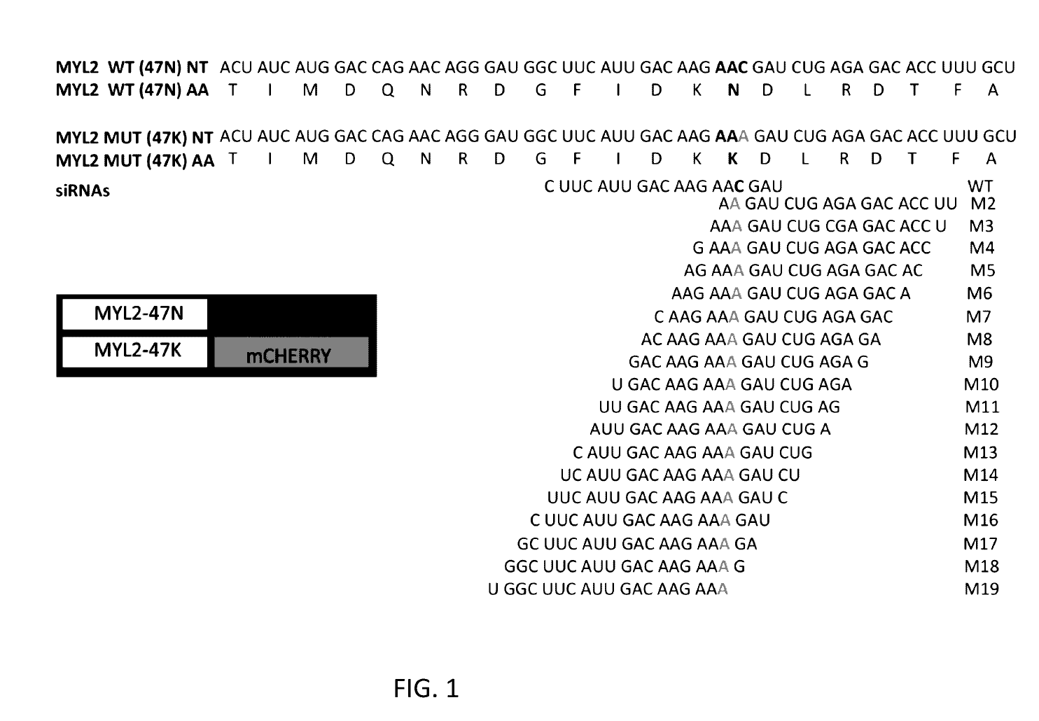

The siRNA duplexes were designed using siRNA walking on the local nucleotide sequence of the MYL2 gene to screen all possible target sequences containing the 47K mutation and synthetized by Protein and Nucleic Acid Facility of Stanford University. See FIGS. 1, 27 and 28. The sense strands are of 19mer length and has incorporated 2-O-methyl modification in each third nucleotide base to increase backbone resistance against endonucleases, while antisense strands are of 21mer length including 2 nucleotide overhangs at the 3′ end, it also contains a phosphate group at the 5′ end, and 2-O-methyl modifications each third base. The 2′-O-methyl modifications play an important role in the stabilization of the mature siRNAs. The siRNAs targeting 403Q mutation were generated using the same strategy.

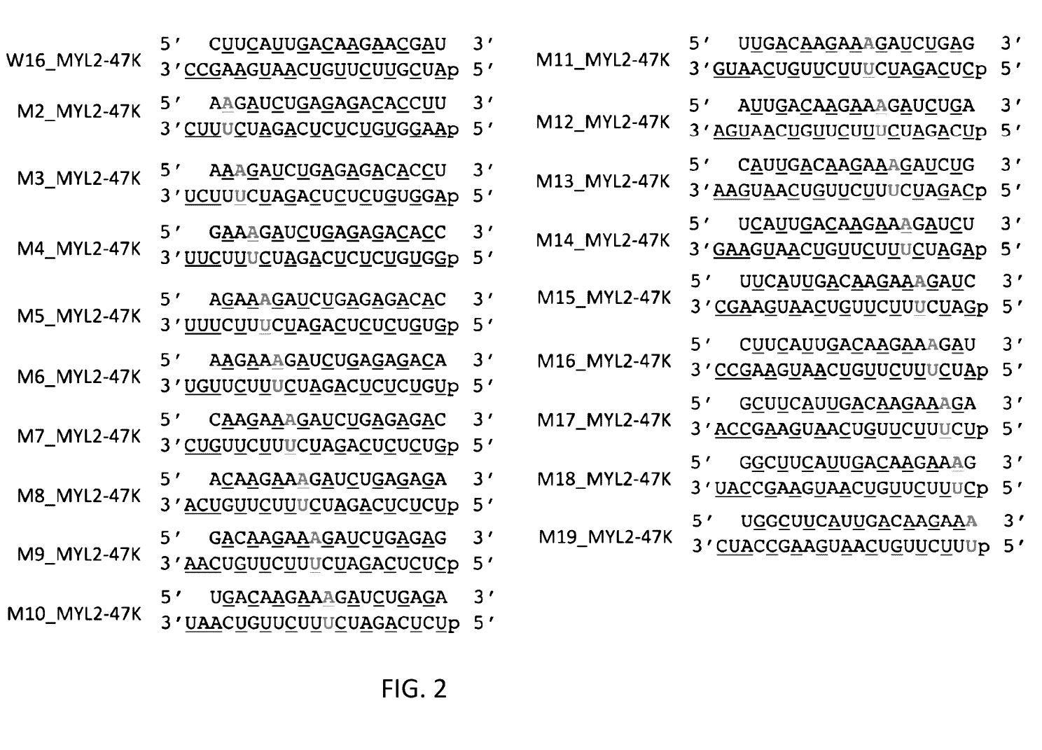

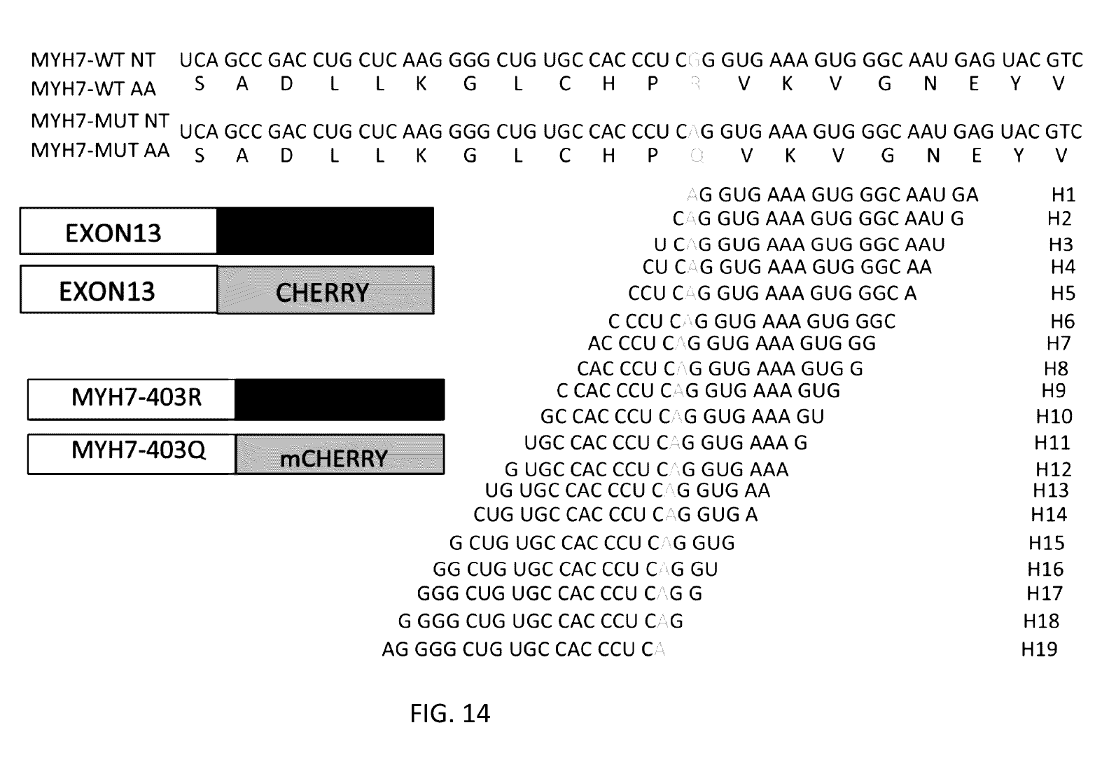

To assist in understanding of embodiments of the present invention, shown in FIG. 1 are small interference RNAs (siRNAs) sequences (WT and M2-M19, SEQ ID NOS:6-24) designed to target the single nucleotide variant “A” (SNV-A) highlighted in gray on the mutant MYL2-47K allele according to an embodiment of the present invention. The target mRNA sequences for wild type MYL2-47N and mutant MYL2-K alleles (SEQ ID NOS:2-5) are also shown. Also, shown in FIG. 2 are siRNAs of W16 and M2-M19 (SEQ ID NOS:6-24, SEQ ID N0:27, and SEQ ID NOS:88-105. Underscored nucleotides contain methyl groups. Antisense strands contain three nucleotide overhangs at the 3′ end and a phosphate group at the 5′ end according to an embodiment of the present invention. Also, shown in FIG. 14 are siRNAs sequences (H1-H19, SEQ ID NOS:44-62) designed to target the single nucleotide variant “A” (SNV-A) of the human MYH7-R403Q allele mutant. The target mRNA sequences for wild type MYH7-403R and mutant MYH7-403Q alleles (SEQ ID NOS:40-43) are also shown.

Short Hairpin RNA (shRNA) Design

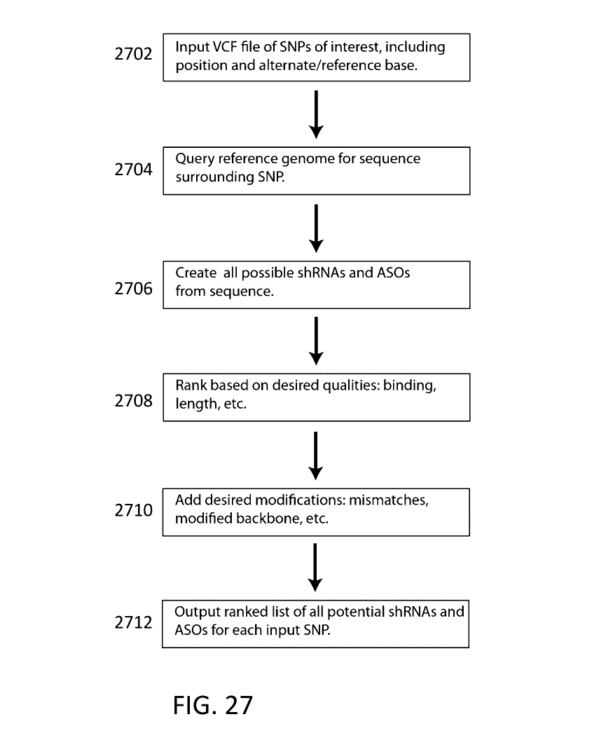

For shRNA design (see also FIGS. 27 and 28), the nucleotide sequence of the sense strand of MYL2-siRNAs was used as template. The 5′ end of the sense strand contains a phosphate group and the restriction site Bbs I, while the 3′ end connect the loop to the 5′ end of the antisense strand (see FIG. 6). The antisense strand contains BbsI restriction site at the 3′ end. The complementary strand contains a C at the 3′ end and a phosphate group at the 5′ end. Two types of loop sequences were used: Loop 8 (CAAGCTTC) and loop 12 (CTTCCTGTCAGA, SEQ ID NO:1). Loop 8 and loop 12 contains a Hind III and HpyCH4 III restriction site respectively.

Cloning of hMYL2 in pEGFP-N1 and pmCherry and Site Directed Mutagenesis

The gene encoding cardiac myosin regulatory light chain was purchased from PlasmID: HsCD00041794 in pDONR221 vector (Boston). pDONR221 was digested with KpnI/AgeI to remove MYL2 wild type gene and subsequently cloned into the pEGFP-N1 (Clontech) vector using the same restriction sites resulting in MYL2-47N. The mutant (MYL2-47K) was generated by site directed mutagenesis using the following primers: sense primer 5′-ATGGCTTCATTGACAAGAAAGATCTGAGAGACACCTTTG-3′ (SEQ ID NO:68) and antisense primer 3′-CAAAGGTGTCTCTCAGATCTTTCTTGTCAATGAAGCCAT-5′ (SEQ ID NO: 69).

After Dpn I digestion, transformation was carried out using XL-Blue competent cells followed by plasmid extraction and sequencing. The mutant was removed by restriction digestion of pDONR221 with XmaI/XhoI and subsequently cloned into pDSRed/mCherry (Clontech).

Cloning of Exon13 (hMYH7) in pEGFP-N1 and pmCherry and Site-Directed Mutagenesis

Human genomic DNA was isolated from normal-person blood. A100 nt region of the wild type MYH7 gene surrounding position 403Q was amplified using the forward primer 5′-GACGGTACCCCATGTACCTCATGGGGCTGA-3′ (SEQ ID NO:70) and reverse primer 5′GCGACCGGTTGCTGGACATTCTGCCCCTTGG-3′ (SEQ ID NO:71). The primer was modified to contain KpnI and AgeI cloning sites and introduced into pEGF-N1 (Clontech) resulting in MYH7 (exon13)-403Q. The mutant was obtained by PCR mutagenesis using the following primers: sense primer 5′-CACCCTCAGGTGAAAGTGG-3′ (SEQ ID NO:72) and antisense primer 5′-CCCACTTTCACCTGAGGGT-3′ (SEQ ID NO:73). Subsequently, the fragment was introduced into pmCherry-N1 (Clontech), resulting in MYH7 (exon13-403Q (mut) fused to mCherry reporter gene.

Cloning of Partial Sequence of hMYH7 in pEGFP-N1 and pmCherry

To construct the plasmid vectors pMYH7-403R-GFP and pMYH7-403Q-mCherry, a 1.5 kb partial sequence of MYH7 gene was amplified by PCR using template plasmid beta808 and primers:

| | |
|---|---|
| MYH7_Hind III Forward | |
| 5′-AAGCTTATGGGAGATTCGGAGATGG-3′ | (SEQ ID NO: 74) |
| and | |
| MYH7_AgeI Reverse | |
| 5′-ACCGGTACAAACATGTGGTGGTTG-3′. | (SEQ ID NO: 75) |

The 1.5 kb fragment was cloned into HindIII/AgeI restriction sites of pGFP-N1 (Clontech). For the mutant, the same strategy was used into pmCherry-N1 (Clontech).

Cloning of Short Hairpin RNAs (shRNAs) in pAAV RSV-Cerulean Plasmid

Sense and antisense oligonucleotides were resuspended at the same molar concentration using annealing buffer (10 mM Tris, pH 7.5-8.0, 50 mM NaCl, 1 mM EDTA). Annealing was carried out at 95° C. for 10 minutes and slowly cooled until reaching room temperature, and then stored at 4° C. The pAAV RSV-cerulean plasmid (SEQ ID NO:121) was digested with BbsI enzyme. Ligation of annealed oligonucleotides into the Bbs I restriction site of the pAAV RSV-cerulean plasmid was carried out using T4 ligase (NEB) followed by transformation and miniprep (Qiagen). shRNA cloning was confirmed by double digestion with Hind III and Nhe and sequencing.

Cloning of M7.8L Short Hairpin RNAs in pRSV eGFP-T2A-Fluc2

The plasmid scAAV H1-M7.8L RSV cerulean was double digested with EcoRV and MfeI. The digestion produced three DNA fragments of 3226 bp, 1273 bp and 323 bp. The 323 bp DNA band was extracted from agarose gel and purified (Qiagen). The 323 bp DNA fragment contains H1 promoter, M7.8L shRNA and partial sequence on RSV promoter. The plasmid pRSV eGFP-T2A-Fluc2 (SEQ ID NO:123) was linearized by double digestion with SnaBI and MfeI restriction enzymes. Ligation using T4 ligase was carried out at room temperature for three hours followed by transformation and miniprep. Plasmid DNA was digested with XhorMfeI and sequencing was carried out to confirm the cloning.

### AAV9 Virus Production Expressing Short Hairpin RNAs

Cells AAV-293 cells (Stratagene) were cultured in DMEM containing 10% fetal bovine serum (Invitrogen) in T-225 cm2 for growth and expansion. After cells reached 80% confluency, HEPES was added to buffer pH (0.4 ml 1.0 M HEPES to 36 ml media to result in 10 mM HEPES). Mix A=Plasmid DNA (27□□g of each plasmid) of adenovirus helper, pladeno5; AAV helper, pAA-2/9-731 and AAV vector, pAAV shRNA RSV cerulean were mixed in a 0.3 M CaCl2 solution. Mix B=In a 50 ml falcon tube 4 ml of 2×HBS (280 mM NaCl, 1.5 mM Na2HPO4, 50 mM HEPES, pH 7.05). A and B were mix by gently pipetting and immediately added to the cell media. Media was mixed by cross swirling and incubated during 18 hours. Media was changed after incubation to remove CaPO4/DNA precipitate and continue incubation for 72 hours for virus production. Media was removed from T-225 flask and 5 ml of PBS containing 10 mM EDTA was used to remove the cells. Flasks were bang gently to dislodge the cells and washed with PBS to collect the rest of the cells. Cells were centrifuged at 2700 rpm for 15 minutes at 4° C. Supernatant was removed and the cell pellet was resuspended in 1 ml freezing buffer (150 mM NaCl, 20 mM TRIS pH 8.0, 2 mM MgCl2) and transferred to 1.5 ml Eppedorf tube. The tube was incubated at −80° C. for 15 minutes. Samples were thaw at 42° C. in a water bath for a total of 3 freeze-thaw cycles. After cell lysis, DNA digestion was carried out with benzonase (250 unit/□l) to a final concentration of 50 units/ml crude lysate and incubated at 37° C. for 30 minutes. Cell debris was removed by centrifugation at 13,500×g (11,100 rpm in Sorvall legend centrifuge) for 30 minutes at 4° C. Virus purification was carried out with different iodixanol gradients. Gradients were prepared in a 5.5 ml thick-walled polycarbonate tubes as follows: 1.5 ml of 60%, 1.0 of 40%, 1.0 ml of 25%, and 1.0 ml of 15% iodixanol solution (heaviest layer in the tube first). To the top of each iodixanol gradient 1.0 ml crude lysate was added. Ultracentrifugation was carried out at 66,100 rpm (400,000×g) at 10° C. for 2 hours in T-1270 rotor in Thermo WX Ultra centrifuge. The top 3 layers (0, 15, 25%) were collected avoiding the clear 40% layer (AAV is in the 40% iodixanol). Iodixanol was removed by ultracentrifugation using 100 kDal molecular weight cut off filters (Millipore). Centrifugation was repeated at 4000 rpm at 4° C. until the sample reached a volume of ˜200 ul. Ultracentrifugation was repeated one more time at 13500×g (12000 rpm in Eppendorf 5424 centrifuge) at 20° C. until the volume was ˜20-100 □l and transferred to a 1.5 ml Eppendorf tube. Genomic titer and infectious titer were determined using standard protocols.

MYL2-N47K shRNAs Screening Using Fluorescent Activated Cell Sorting

Due to the lack of gene silencing in the first cell model, we opted to prepare a second construct containing partial gene sequence of the MYH7 gene. PCR amplification of 1.5 kb DNA fragment was amplified using as template plasmid wild type and mutant beta808. The 1.5 kb fragment was cloned in pEGFP-N1 and pmCherry respectively. The siRNAs were tested on these constructs, showing similar results as the first exon13 cell model. Both cell models showed that H10 and H11 siRNA silence 65% of the 403Q mutation and 20-25% of the wild type allele, which is statistically significant.

siRNA and shRNA Screening Using Fluorescence Activated Cell Sorting

HEK293 cells were co-transfected with plasmids coding the human RLC: pMYL2-47N-GFP and pMYL2-47K-mCherry (100 ng each plasmid). Lipofectamine LTX (Invitrogen) was used to mediate transfection following the manufacturer's instructions. G418 antibiotic was used at 600 ug/ml to generate stably transfected cell lines. Flow activated cell sorting was used to isolate individual clones expressing both GFP and mCherry proteins.

Cells co-expressing MYH7-403R-eGFP and 403Q-mCherry fusion proteins were generated using the same strategy Stable transfected HEK293 cells were transfected with siRNAs duplexes and shRNAs using Lipofectamine RNAiMax (Invitrogen) following the manufacturer's instructions 48 hours post-transfection, cells were harvested and analyzed by flow cytometry (LSR II) to measure knockdown. The data was analyzed using Flowjo 9.2 software.

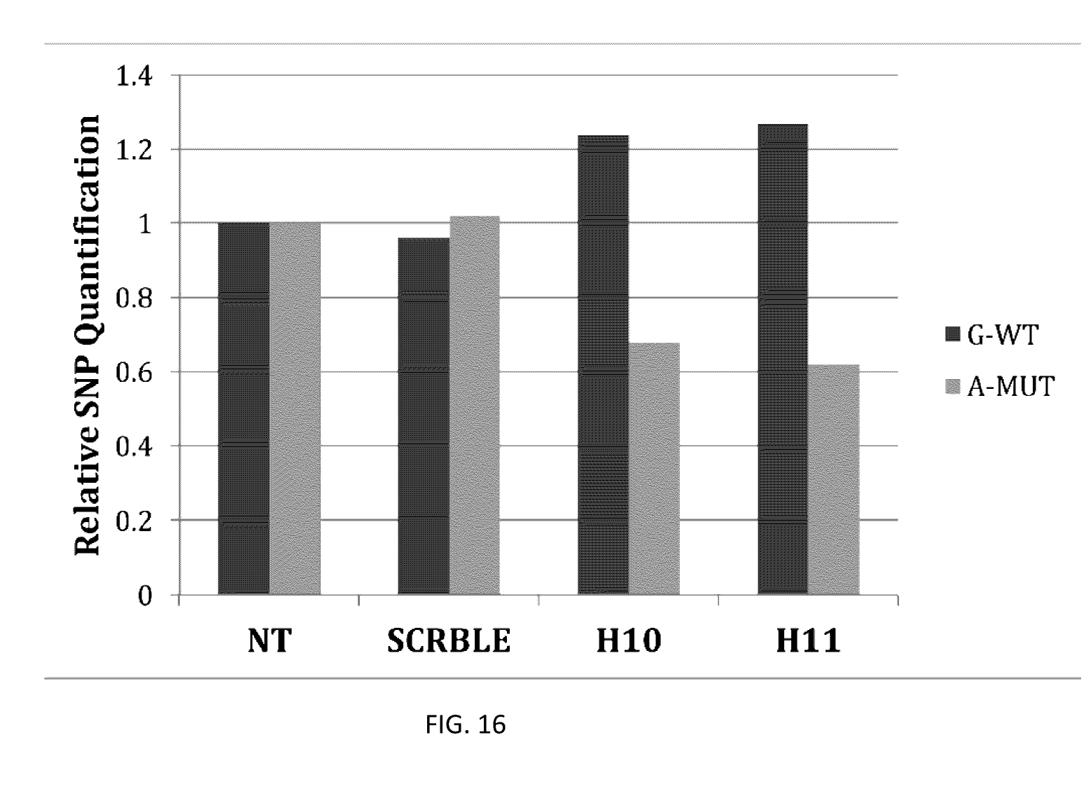

To assist in understanding embodiments of the present invention, shown in FIG. 15 is fluorescence activated cell sorting of the relative GFP and mCherry expression of double stable transfected human embryonic kidney cells containing MYH7-403R-GFP and MYH7-403Q-mCherry and transfected with H10.8L and H11.8L shRNAs. Also, shown in FIG. 16 is relative SNP quantification using pyrosequencing of stable transfected HEK cells with plasmids MYh7-403R-GFP and MYH7-403Q-mCherry and treated with plasmids expressing shRNAs: H10.8L and H11.8L.

Allele Quantitative Polymerase Reaction (qPCR)

RNA isolation (RNeasy Qiagen) and cDNA synthesis (Applied Biosystems) were carried out for allele mRNA quantification using Q-PCR. For mutant detection and wild type discrimination the following reaction was carried out in 20 ul volume: Mutant specific (18mer) primer Forward (0.5 um), Reverse primer (0.5 um), wild-type specific blocker (5 um), Taqman probe containing 6-FAM at the 5′ end and TAMRA at the 3′ end, endogenous control mouse and human GAPDH and Taqman Universal PCR Master mix, (No AmpErase UNG. Par Number 4324018). For wild type detection and mutant discrimination the reaction was as follows: Wild type specific Forward, Reverse primer (0.5 um), mutant specific blocker (5 um), Taqman probe containing 6-FAM at the 5′ end and TAMRA at the 3′ end, endogenous control mouse and human GAPDH and Taqman Universal PCR Master mix, (No AmpErase UNG). The Q-PCR reaction was performed by 95° C./10′ Hold, 95° C./30″, 50° C./1′, 60° C./1′ 35 cycles.

### Genotype Determination of Human Transgenic RLC-N47K Mice

To identify the human transgenic RLC-47N, RLC-47K and RLC-N47K mice, primers: MYL2-Forward primer (5′-AAGAAAGCAAAGAAGAGAGCCGGG-3′, SEQ ID NO:76) and MYL2-Reverse primer 5′-TGTGCACCAGGTTCTTGTAGTCCA-3′, SEQ ID NO:77) were used to amplify a 450 bp fragment. PCR fragments were digested with Bgl II restriction enzyme for identification of the wild type and mutant alleles. The Bgl II restriction enzyme cuts the mutant MYL2 gene once, yielding two bands of 325 bp and 125 bp. Bgl II does not cut the wild type MYL2 gene. To identify both alleles (mutant and wild type) in the mice, three bands are produced: the uncut wild type fragment (450 bp) and the two mutant fragments (325 bp and 120 bp). The primers are designed to discriminate the mouse RLC.

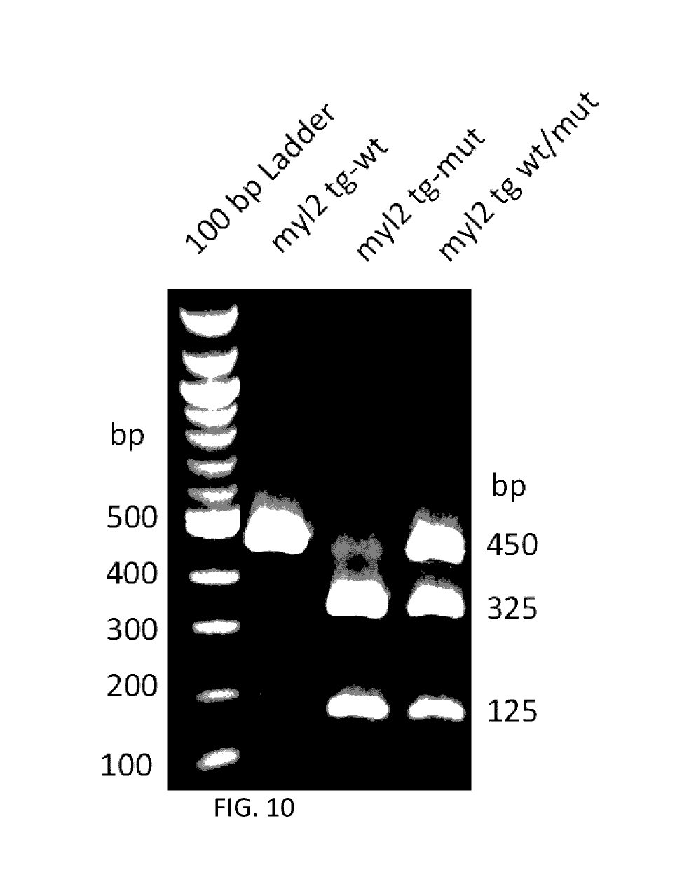

To assist in understanding of the present invention, shown in FIG. 10 is genotype determination of human MYL2-N47K mouse model by PCR and Bgl II restriction digestion.

### Neonatal Cardiomyocyte (NCM) Cell Isolation and Culture

Mice were put to sleep with mild hypothermia in accordance to the administrative panel on laboratory animal care (APLAC) standard protocols from Stanford University. Neonatal cardiomyocytes (NCM) were isolated from three days old mice hearts dissected in 10 ml ice-cold calcium and bicarbonate free hanks with HEPES (CBFHH) buffer.

Hearts were digested in a tube containing 5 ml of Papain solution (1 vial of papain and 1 vial of DNase from Worthington Papain Dissociation System in 10 ml CBFHH buffer) at 37° C. for 15 minutes with mild shaking. Heart tissue was triturated by gently pipetting and centrifuged at 1000 rpm for 5 minutes. This step was repeated until the tissue was dissolved. Digestion was stop by adding same volume of pre-warmed fetal bovine serum (FBS). The cell solution was filtered through 40 um nylon cell strainer and spin at 1000 rpm for 5 minutes at room temperature. Cells were suspended in 10 ml of myocyte media (1×Dulbecco's Mod. Eagle Medium (DMEM) media containing 5% FBS, 10% Horse serum and Penr/Strepr/Amphr) and transferred to an uncoated petri dish. The plate was incubated for an hour at 37° C. in a CO2 incubator for fibroblast attachment. Myocytes in suspension were collected and spin at 1000 rpm for 5 minutes at 25° C. and suspended in 5 ml of myocyte media. Cells were count and plated in laminin-coated dishes with desired density. Next days media was changed with myocyte media containing 1 μm (final concentration) of the anti-mitotic of cytosine beta D-arabinofuranoside. Media was changed every 3 days. Cells were fed by substituting only half of media to not expose cells to the air.

Optimization for Relative mRNA Quantification Using Plasmid Mixture

To test and optimize allele discrimination for mRNA quantification, we generated a mixture (1:1 ratio) of wild type and mutant plasmid DNA (pMYL2-47N_GFP and pMYL2-47K mCherry respectively). Although it is certain that there is an unequal allelic expression of wild type and mutant alleles in HCM, this mixture represents a heterozygous sample with equal allelic expression of the human regulatory light chain (RLC) for siRNA and shRNA screening. To quantify the mutant allele and discriminate the wild type allele, we designed wild type blocker oligomers of 20mer and 19mer containing a phosphate group at the 3′ end that will interrupt the PCR amplification of the wild type allele (Morlan et. al, 2009). The wild type blockers contain wt-SNV in the middle of the oligomer. We also designed and test different mutant specific forward oligomers of 22mer, 21mer, 20mer, 19mer, and 18mer. These primers have the SNV mutation at the 3′ end (Morlan et. al, 2009). The shortest oligomers (19mer and 18mer) showed more specificity when in the absence of the wild type blocker did not amplify the wild type allele; meanwhile the rest required the presence of the wild type blocker in order to discriminate the wild type allele. The reverse primer is designed to discriminate the mouse RLC. For quantification of the wild type allele and discrimination of the mutant allele, we did the opposite, the wild type blocker became the wild type specific forward primers and the mutant specific primers became the mutant specific blockers. The reverse primer and the Taqman probe is the same for both mRNA quantification reactions (see Table I).

TABLE 1
Primers and probe used for MYL2-N47K allele
quantitative PCR using blockers. Reverse
primer discriminates the
mouse gene background.

| PRIMERS | SEQUENCES (5′-3′) |
|---|---|
| P3 | ATGGCTTCATTGACAAGAAA |
| (Fwd-20mer) | (SEQ ID NO: 78) |
| P4 | TGGCTTCATTGACAAGAAA |
| (Fwd-19mer) | (SEQ ID NO: 79) |
| P5 | GGCTTCATTGACAAGAAA |
| (Fwd-18mer) | (SEQ ID NO: 80) |
| PWT-Fwd | GGATGGCTTCATTGACAAGAAC |
| | (SEQ ID NO: 81) |
| PWT-Rev | TTCCTCAGGGTCCGCTCCCTTA |
| | (SEQ ID NO: 82) |
| B1 | TGACAAGAACGATCTGAGA-PO4 |
| (wild type | (SEQ ID NO: 83) |
| blocker 1) | |
| B3 | ATGGCTTCATTGACAAGAAC-PO4 |
| (Mutant | (SEQ ID NO: 84) |
| Blocker 3) | |
| B4 | TGGCTTCATTGACAAGAAC-PO4 |
| (Mutant | (SEQ ID NO: 85) |
| Blocker 4) | |
| B5 | GGCTTCATTGACAAGAAC-PO4 |
| (Mutant | (SEQ ID NO: 86) |
| Blocker 5) | |
| MYL2 | FAM-TGGATGAAATGATCAAGGAGGCTCCG-TAMRA |
| Taqman Probe | (SEQ ID NO: 87) |

R403Q iPSc Reprogramming

Generation, maintenance, and characterization of patient-specific iPSC lines were performed as previously described (Lan et al., 2013). Briefly, the skin biopsy was minced in collagenase (1 mg/ml in Dulbecco's modified Eagle medium (DMEM), Invitrogen, Carlsbad, Calif.) and digested in 37° C. for 6 hours. The derived dermal fibroblasts were plated and maintained with DMEM containing 10% FBS (Invitrogen), and pen-strep (Invitrogen), at 37° C., and 5% CO2 in a humidified incubator. Cells within five passages were reprogrammed using lentiviral vectors individually expressing OCT4, SOX2, KLF4 and c-MYC. Six days after transduction, the cells were re-plated on Matrigel-coated tissue culture dishes with mTeSR1 media (StemCell Technology, Vancouver, Canada). The iPSC colonies appeared after two weeks of culture were manually picked for expansion.

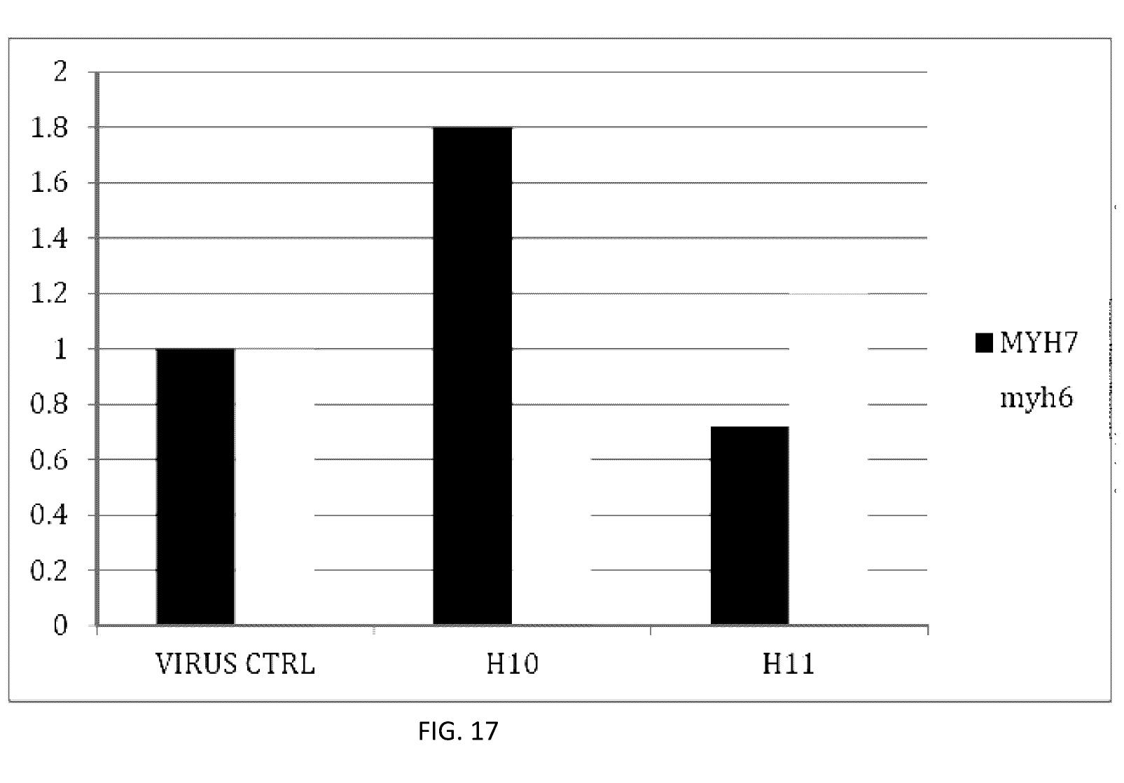

To assist in understanding of the present invention, shown in FIG. 17 is relative mRNA quantification of hMYH7 and hMYH6 of human R403Q cardiomyocytes differentiated from induced pluripotent cells (iPSc) and transduced with AAV9 expressing H10.8L and H11.8L shRNAs.

### Human Pluripotent Cell Culture

All pluripotent cultures were maintained at 37° C. in a New Brunswick Galaxy 170R humidified incubator (Eppendorf, Enfield, Conn., USA) with 5% CO₂ and 5% O₂ controlled by the injection of carbon dioxide and nitrogen. All primary and differentiation cultures were maintained at 5% CO₂ and atmospheric (21%) O₂. The hESC line H7 (WA07) (Thomson et al., 1998) was maintained on 6-well tissue culture plates (Greiner, Monroe, N.C., USA) coated with or 1:200 Growth-factor reduced Matrigel (˜9 μg/cm2, BD Biosciences, San Jose, Calif., USA) in mTeSR1 (Stem Cell Technologies, Vancouver, BC, Canada). Media was changed every day. Cells were passaged every 4 days with 0.5 mM EDTA (Life Technologies, Carlsbad, Calif., USA) in D-PBS without Ca2+/Mg+ (Life Technologies) for 7 minutes at RT and split 1:8 to 1:10. Cell lines were used between passages 20 and 70. All cultures were maintained with 2 mL media per 10 cm2 of surface area or equivalent. All cultures were routinely tested for mycoplasma using a MycoAlert Kit (Lonza, Allendale, N.J., USA).

Cardiac Differentiation of hESC

The hESC split at 1:10 ratios using EDTA as above were grown for 4 days, at which time they reached ˜85% confluence. On day 0, the differentiation media was changed to RPMI+B27-ins, consisting of RPMI 1640 (11875) supplemented with 2% B27 without insulin (0050129SA, Life Technologies). The media was changed every other day (48 hours). For days 0-2, the media was supplemented with 6 μmol/L CHIR99021 (LC Labs, Woburn, Mass., USA). On day 2, the media was changed to RPMI+B27-ins supplemented with 5 μmol/L IWR-1 (Sigma-Aldrich, St Louis, Mo., USA). The media was changed on day 4 and every other day for RPMI+B27-ins. Contracting cells were noted from day 7. On day 15, cells were dossicated with 10 minutes TrypLE Express (Invitrogen) at 37° C. and re-plated on Matrigel-coated coverslips for further analysis.

### Results and Discussion

Identification of siRNAs that Silence MYL2-47K Mutation.

Fluorescence activated cell sorting was carried out to screen nineteen siRNAs that target MYL2-N47K mutation in a cell system model containing the MYL2 wild type and mutated alleles fused to green and cherry fluorescent reporters, respectively, into human embryonic kidney cells (HEK293).

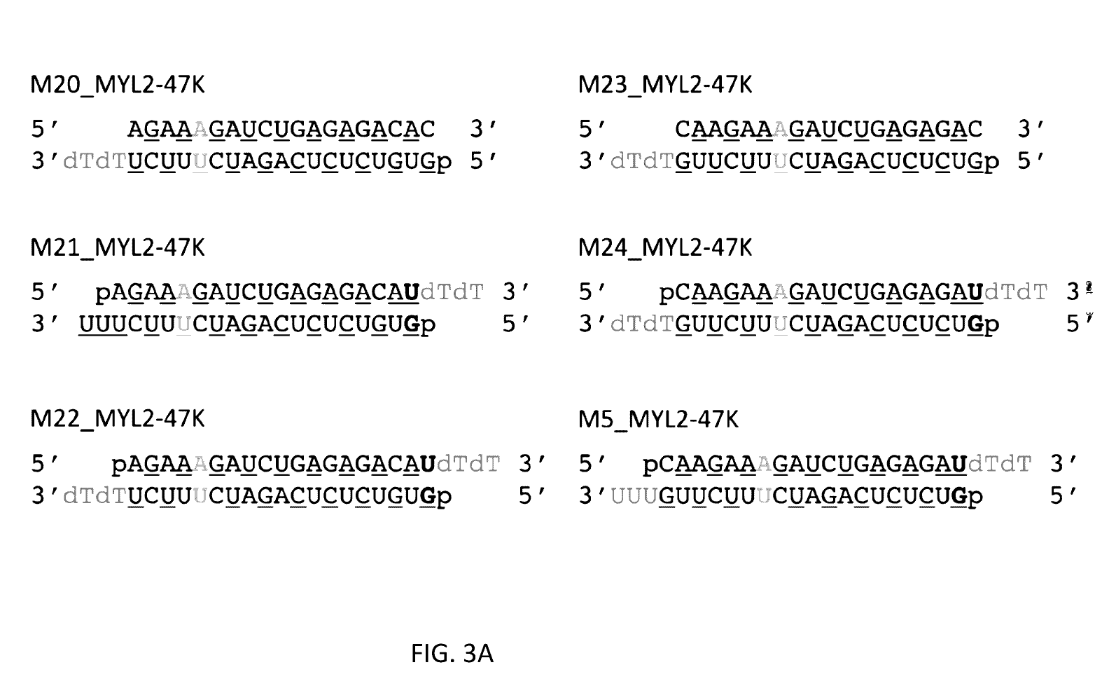

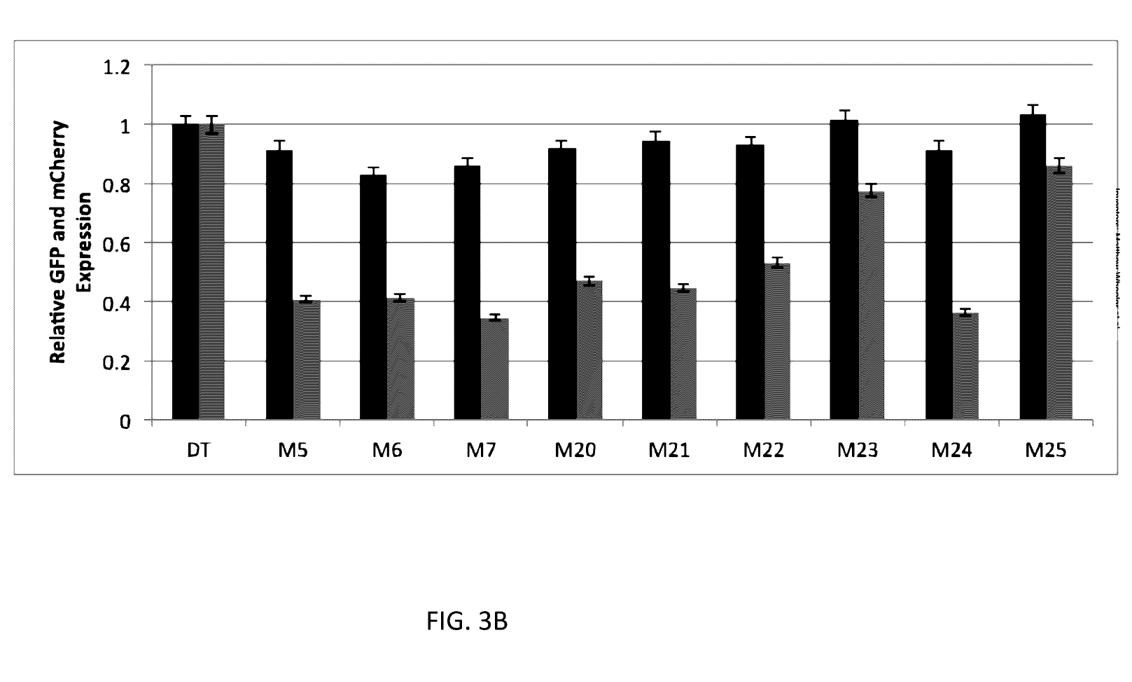

Control siRNA (W16) silence 80% of the wild type allele and 40% of the mutant allele suggesting that a single nucleotide is impossible to discriminate 100% the wild type allele (Table 2). M2, M3 and M4 siRNAs silence both alleles at the same percentage level (˜30%). However M5 and M6 showed statistical significant results when they showed knockdown between 55-60% of the mutant allele and 20-25% of the wild type allele (see FIG. 3B).

FIGS. 3A and 3B show relative expression of wild-type MYL2-47N and mutant MYL2-N47K in the presence of different siRNAs according to embodiments of the present invention. FIG. 3A shows sequences of siRNAs M20-M25 (SEQ ID NOS:25-34). Underscored nucleotides contain methyl groups. Antisense strands contain deoxy-thymidine overhangs at the 3′ end and a phosphate group at the 5′ end according to an embodiment of the present invention. The SNV is highlighted. FIG. 3B shows results for siRNAs M5-M7 and M20-M25 according to an embodiment of the present invention.

The M7 siRNA showed more interesting statistical results and is a promising hit for a genetic drug since it silences 55% of the mCherry expression and 7% of the green expression. The M9 siRNA showed up-regulation of the mutant allele suggesting that it can be used as a genetic drug to induce HCM with the possibility of creating an HCM model. The M8 and M10-M19 showed silencing of both alleles from 60-80% suggesting that a point mutation starting at the nucleotide position 10 is not either mutant or wild type specific. Overall, the M7 siRNA is the most promising hit for a genetic drug for gene therapy for HCM.

To increase their specificity and efficacy of M5, M6 and M7 siRNAs, chemical modifications such as Non-pair Watson crick modifications in the guide strand, dT overhangs at the 3′ end and phosphate groups in the 5′ ends were made on these three siRNAs (see FIGS. 2, 3A, and 4). For example, FIG. 2 shows siRNAs of W16 and M2-M19 (SEQ ID NOS:6-24, SEQ ID NO:27, and SEQ ID NOS:88-105.

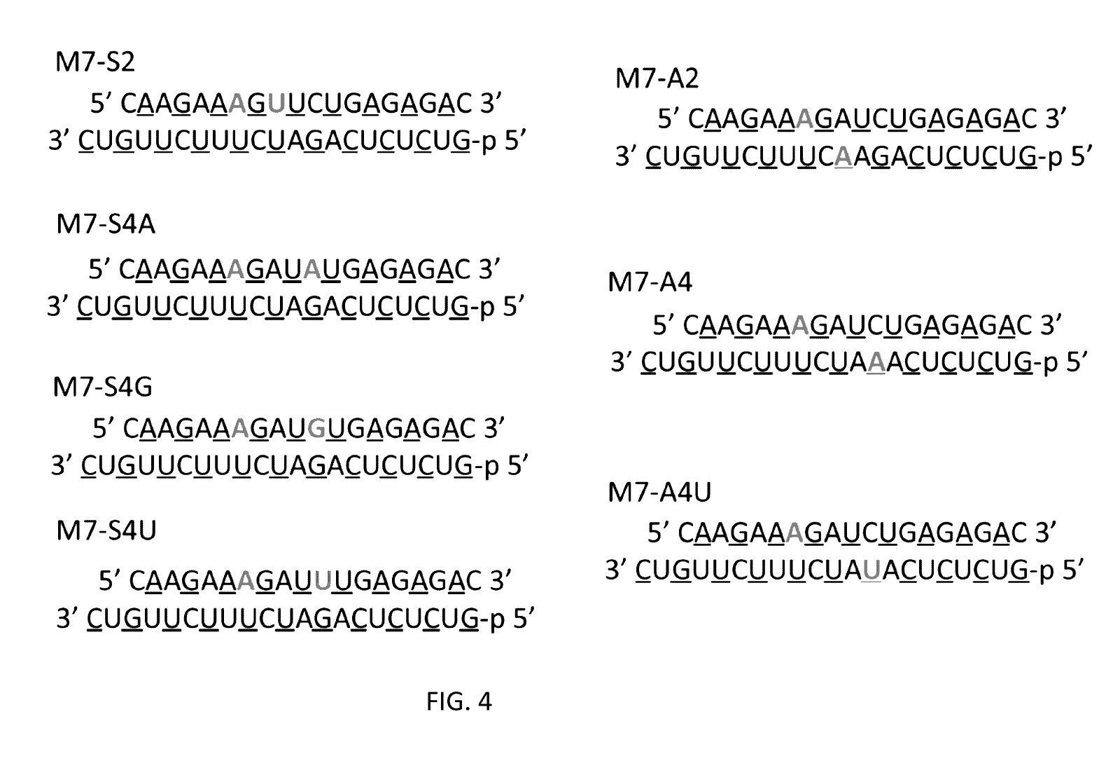

Underscored nucleotides contain methyl groups. Antisense strands contain three nucleotide overhangs at the 3′ end and a phosphate group at the 5′ end according to an embodiment of the present invention. FIGS. 3A and 3B show relative expression of wild-type MYL2-47N and mutant MYL2-N47K in the presence of different siRNAs according to embodiments of the present invention. FIG. 3A shows sequences of siRNAs M20-M25 (SEQ ID NOS:25-34). Underscored nucleotides contain methyl groups. Antisense strands contain deoxy-thymidine overhangs at the 3′ end and a phosphate group at the 5′ end according to an embodiment of the present invention. The SNV is highlighted. FIG. 3B shows results for siRNAs M5-M7 and M20-M25 according to an embodiment of the present invention. And, FIG. 4 shows different modifications of the M7 siRNA (SEQ ID NOS:106-119). Underscored nucleotides contain methyl groups. Antisense strands contain three nucleotide overhangs at the 3′ end and a phosphate group at the 5′ end according to an embodiment of the present invention. The SNV is highlighted. Each siRNA contains non-pair Watson-crick modifications.

Highly modified siRNAs showed increased stability but lacked the ability to silence either wild type or mutant alleles. siRNAs containing additional mismatches also did not show improved efficacy or specificity compared to the original siRNAs.

For the human 403Q mutation, we generated two types of cell B-MYH7 models for in vitro studies, one with the exon 13 and a second with the partial gene sequence of MYH7 with their respective mutants. For the first cell model, exon 13 was amplified from human genomic DNA and cloned into pEGFP-N1 and pmCherry. Site directed mutagenesis was done for exon13-mCherry construct to create 403Q mutant. Both plasmids Exon13(403R)-GFP and Exon13(403Q)-mCherry were co-transfected in HEK293 cells. Nineteen siRNAs targeting 403Q mutation were tested by flow cytometry. Changes of green and red fluorescent proteins was not significant for most of these siRNAs suggesting that messenger RNA (mRNA) is very stable and difficult to be degraded by endonucleases of the RNAi pathway. However a possible hit, H10 siRNA decrease 70% of -mCherry expression, while GFP by 10%.

TABLE 2
Fluorescence Activated Cell sorting of the relative GFP and mCherry
expression of double stable transfected Human embryonic kidney cells
containing MYL2-47N-GFP and MYL2-47K-mCherry.

| | Relative GFP | Relative mCherry |
|---|---|---|
| siRNA | expression | expression |
| DT | 1 | 1 |
| W16 | 0.23 | 0.59 |
| M2 | 0.50 | 0.63 |
| M3 | 0.52 | 0.61 |
| M4 | 0.68 | 0.59 |
| M5 | 0.76 | 0.45 |
| M6 | 0.83 | 0.53 |
| M7 | 0.85 | 0.48 |
| M8 | 0.83 | 0.79 |
| M9 | 0.88 | 1.2 |
| M10 | 0.35 | 1.18 |
| M11 | 0.38 | 1.48 |
| M12 | 1.12 | 0.74 |

Identification of Two Short Hairpin RNAs: M7.8L and H11.8L that Silence Human MYL2-47K and MYH7-403Q Mutations.

Two small shRNAs libraries were prepared in a double strand adeno-associated (AAV) viral vector to target human mutations MYL2-47K and MYH7-403Q. In parallel, HEK293 cells were stably transfected and sorted to express equally wild type and mutant alleles attached to green and mCherry fluorescent proteins respectively and generate allele cell models: MYL2-N47K and MYH7-R403Q, each one for their respective libraries. shRNA libraries were screen using these cells models by transfecting them with lipofectamine and analyzed by Flow activated cell sorting (FACS) for specificity and efficacy. We have identified three shRNAs M5.8L, M6.8L and M7.8L with high efficacy and specificity that targets MYL2-47K mutation without affecting the wild type allele MYL2-47N (FIG. 6). For example, FIG. 6 shows fluorescence activated cell sorting (FACS) of stable transfected HEK cells with MYL2-47N-GFP and MYL2-47K-mCherry and transfected with plasmid expressing shRNAs: M5.8L, M6.8L and M7.8L.

However during experiments, M7.8L shRNA showed more consistency silencing 65% of the MYL2-47K mutation and 10% of the wild type allele. We have also identified two shRNAs, H10.8L and H11.8L that target and silence the human MYH7-403Q mutation 65% and the wild type allele 28% (FIG. 15). For example, FIG. 15 shows fluorescence activated cell sorting of the relative GFP and mCherry expression of double stable transfected human embryonic kidney cells containing MYH7-403R-GFP and MYH7-403Q-mCherry and transfected with H10.8L and H11.8L shRNAs.

The rest of the shRNAs did not show changes of green and red fluorescence. This system worked well for the in vitro shRNA selection; however for our ex vivo and in vivo experiments, it was not ideal. Therefore, we also used quantitative PCR to quantify the mutant allele and discriminate the wild type allele and vice versa using primer blockers. The allele PCR quantification showed similar results to the flow cytometry data.

To assist in understanding embodiments of the present invention, shown in FIG. 5 is the design and cloning of shRNAs in the AAV9 vector pAAV-H1p RSVp-Cerulean. Also, shown in FIG. 6 is fluorescence activated cell sorting (FACS) of stable transfected HEK cells with MYL2-47N-GFP and MYL2-47K-mCherry and transfected with plasmid expressing shRNAs: M5.8L, M6.8L and M7.8L.

### M7.8L Silence MYL2-47K Mutation in Transgenic Neonatal Cardiomyocytes.

Neonatal cardiomyocytes (NCM) were isolated from hearts of three days old transgenic mice expressing human wild type (RLC-47N) and mutant (RLC-47K) regulatory light chain (RLC) and genotyped for identification. After 24 hours culture, cells were transduced with AAV9 expressing M7.8L shRNA and the control virus (not expressing shRNA). Cells were incubated for 72 hours and collected for RT-PCR and Q-PCR. Data was consistent with several of our experiments showing 40-50% of silencing of the mutation (MYL2-47KN) and 10-15% of the wild type (MYL2-47K) allele.

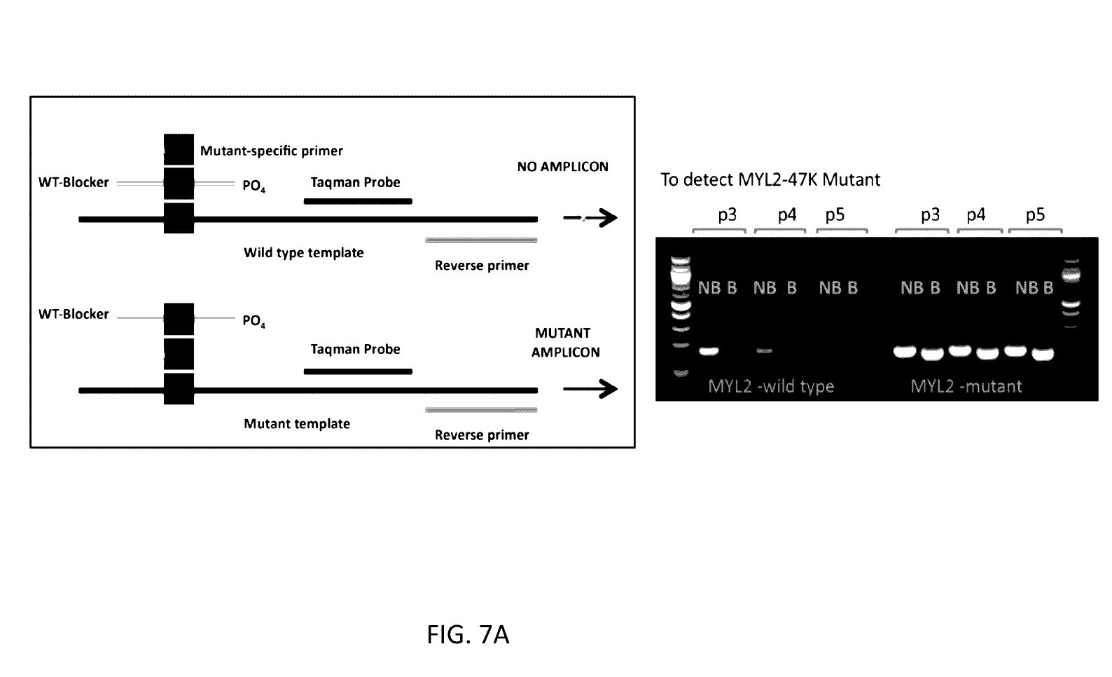

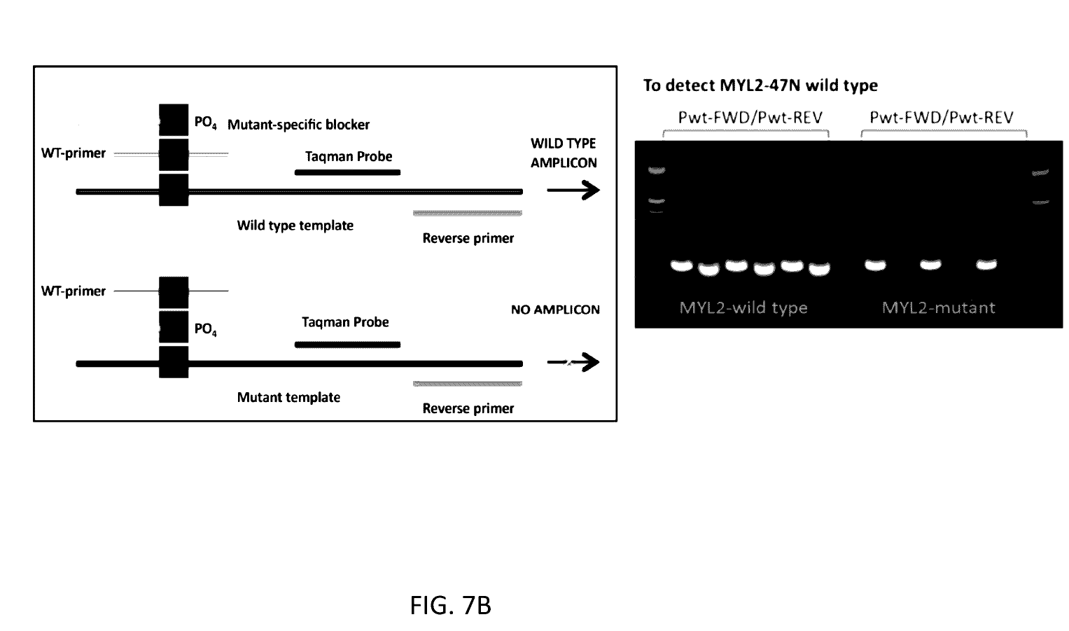

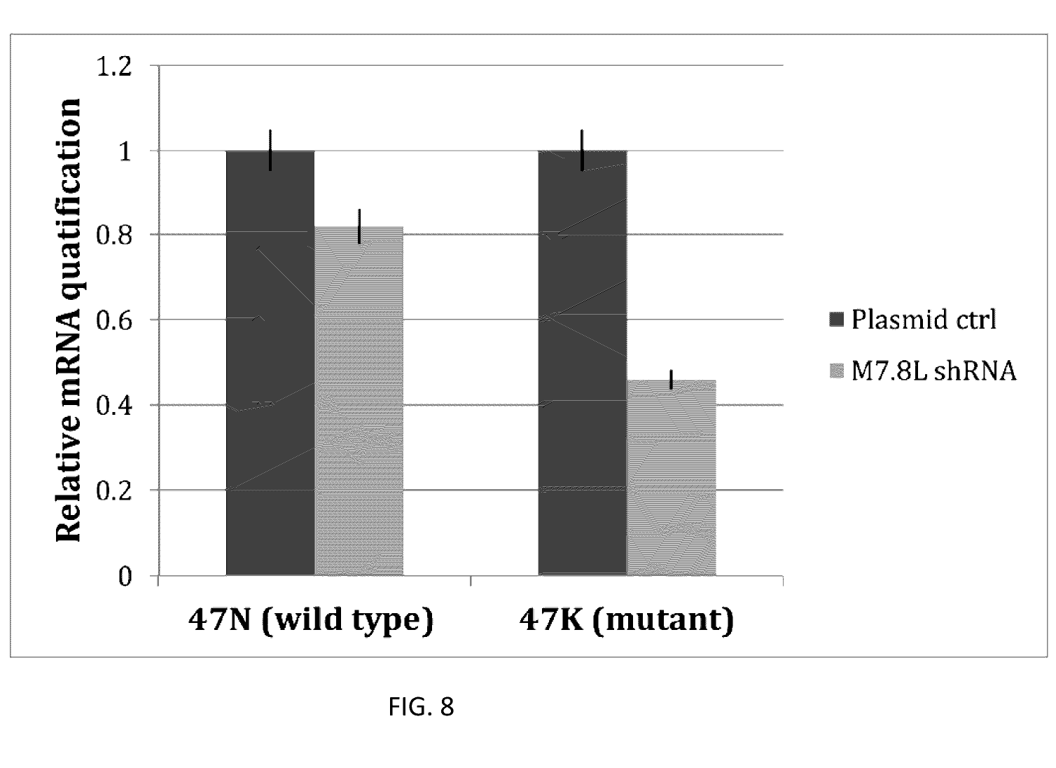

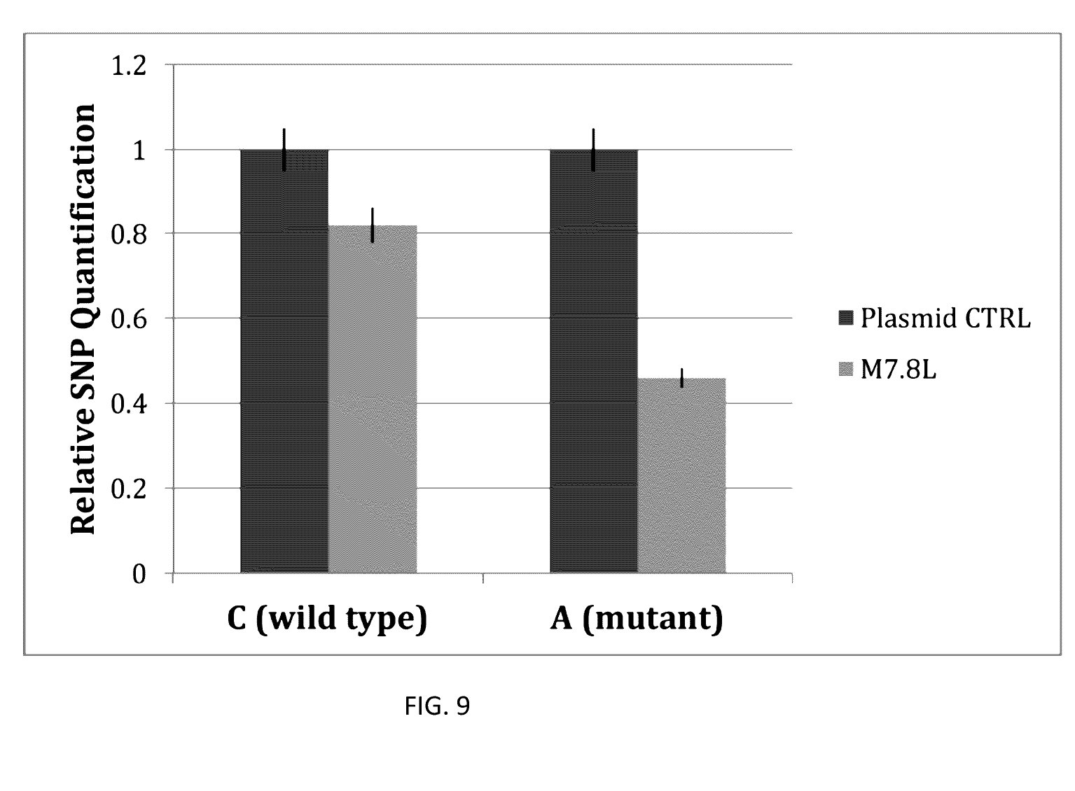

To assist in understanding embodiments of the present invention, shown in FIGS. 7A and 7B is the design of quantitative polymerase chain reaction (q-PCR) assays using a blocker for allele discrimination. FIG. 7A shows amplification of the mutant with blocker (B1) and with no blocker (NB) using wild type (WT) and mutant template. FIG. 7B shows amplification of the wild type using different blockers (B3, B4 and B5) and with no blocker (NB) and WT and mutant template. Also, shown in FIG. 8 is relative mRNA quantification using qPCR (q-PCR system from FIG. 7-8 of stable transfected HEK cells with plasmids MYL2-47N-GFP and MYL2-47K-mCherry and treated with plasmids expressing shRNAs: M5.8L, M6.8L and M7.8L.

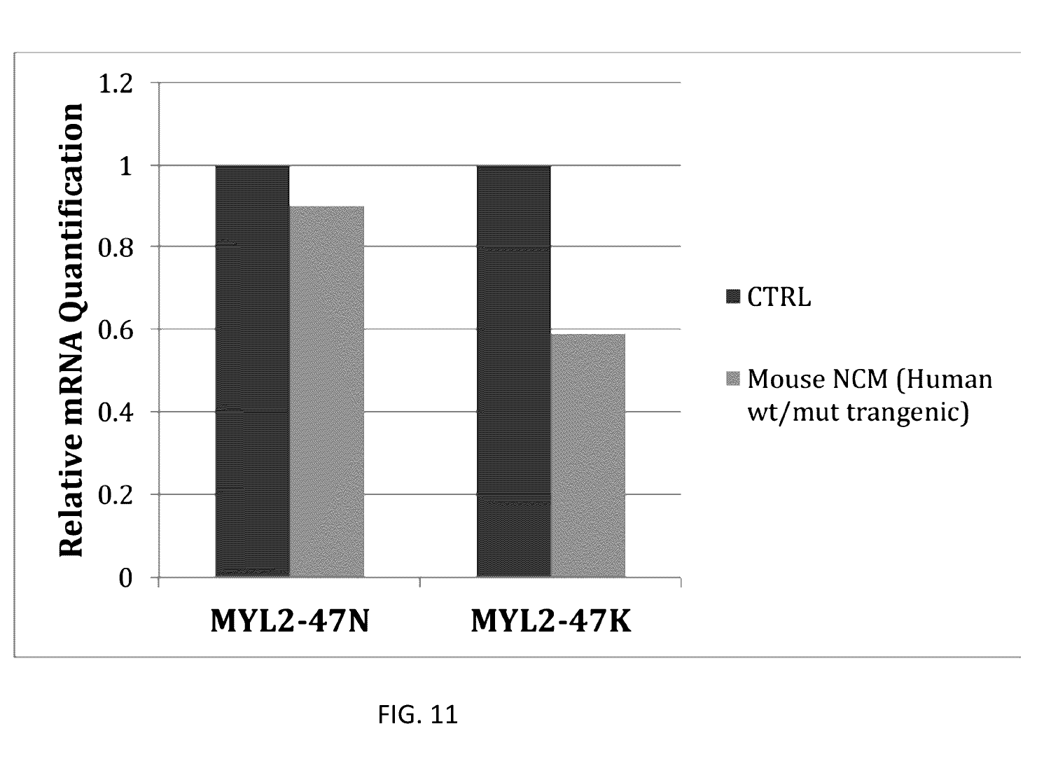

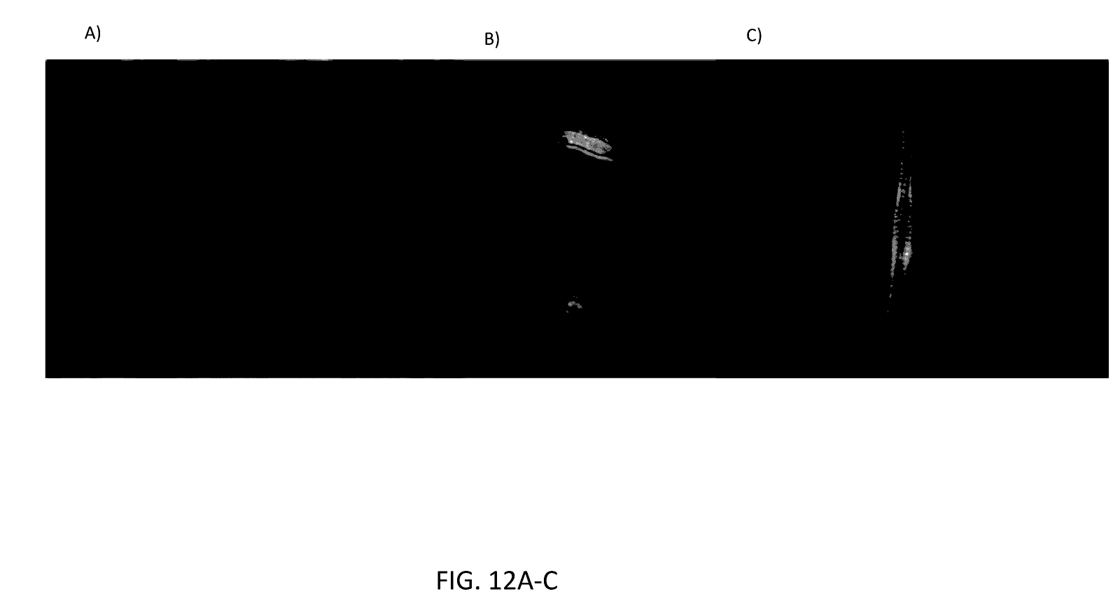

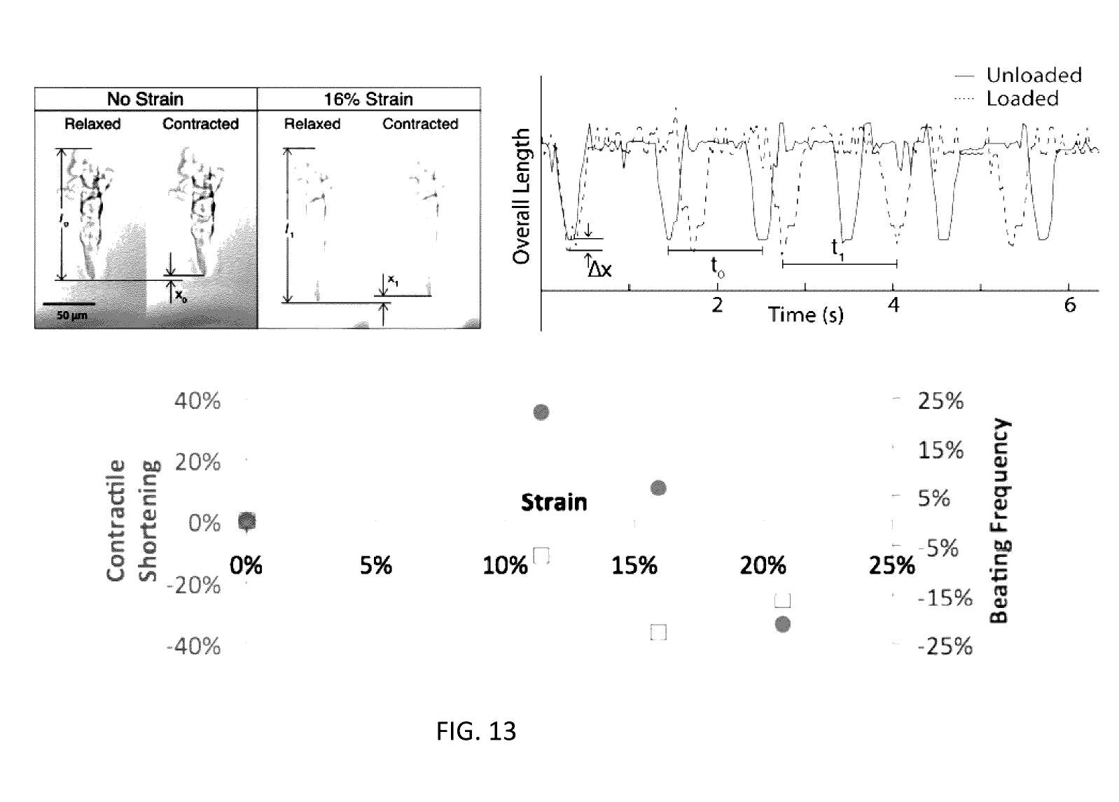

To assist in understanding of the present invention, shown in FIG. 11 is allele quantitative PCR of transgenic neonatal cardiomyocyte cells (NCM) transduced with AAV9 expressing M7.8L shRNA. Also, shown in FIGS. 12A-12C is micropatterning of neonatal cardiomyocytes cultured on a micro stamp. FIG. 12A shows human transgenic neonatal cardiomyocytes transduced with AAV9 expressing M7.8L shRNA and cerulean reporter. FIG. 12B shows that NCM have an elongated shape and sarcomeric organization after cultured on a micro stamp. FIG. 12C shows an image of NCM cultured on a stamp and fixed and stained against alpha-actinin and DNA. Shown in FIG. 13 are contractile studies of elongated cardiomyocytes.

Sarcomeric Organization and Traction Force Microscopy in MYL2-N47K Neonatal Cardiomyocytes Transduced with AAV9 Expressing M7.8L shRNA.

Cells were cultured on 2000 um² rectangular laminin (BD Biosciences) patterns with an aspect ratio of 5:1 on polyacrylamide (PA) substrates to generate an elongated shape and sarcomeric organization, to contract along their main axis and to present aligned sarcomere organization. 1 Gels were fabricated as reported elsewhere. 2 In summary, we mixed PA gel components (12% acrylamide-0.15% N, N-methylene-bis-acrylamide) in DI water and added 50 μl of solution on clean coverslips pretreated with aminopropyltriethoxysilane and glutaraldehyde. Polymerization occurred after we placed on top of the gel component solution a coverslip with stamped patterns on its surface facing the gel. We used ammonium persulfate as a catalyst for gel polymerization and N,N,N,N-tetramethylethylenediamine as an initiator. We patterned coverlips and transferred patterns to gels according to an already published method. 3 In summary, we used soft lithography to fabricate polydimethylsiloxane (Sylgard) microstamps. We flooded the microstamps with 10 □g/mL laminin for 30 minutes, dried microstamps under a stream of N2 and then placed the region coated with protein on top of a pre-cleaned glass coverslip to be placed on top of the gel solution. Once in culture, videos of contractile cardiomyocytes were acquired in brightfield with a high speed CCD camera (Orca-R2 Hamamatsu). We measured the contractile shortening of cardiomyocytes and beat rate with custom ImageJ and Matlab scripts.

Transduction of RLC-N47K Transgenic Mice with AAV9 Expressing M7.8L shRNA.

For in vivo experiments, luciferase AAV9 vector pRSV eGFP-T2A-Fluc2 expressing M7.8L under H1 promoter was used in this study. To clone the H1 promoter together with M7.8L shRNA in the AAV Fluc2 vector, two primers were designed with EcoNI and NheI restriction sites in the 5′ and 3′, respectively.

To assist in understanding of embodiments of the present invention, shown in FIGS. 18A and 18B are AAV9-Luciferase viral vectors expressing M7.8L shRNA under an H1 promoter for in vivo experiments in mice containing human MYL2 wild type and mutant transgenes. FIG. 18A shows a schematic of the pAAV-RSV-eGFP-T2A-Fluc2 vector (SEQ ID NO:123). FIG. 18B shows a schematic of the pAAV-CBA-Fluc vector (SEQ ID NO:122).

H118L shRNA on Differentiated Human Cardiomyocytes.

Skin sample from a patient with 403Q mutation was obtained and reprogramed to generate iPS cells and cardiomyocytes. Differentiated cardiomyocytes from iPS cells growth as monolayer showing a phenotype like the neonatal cardiomyocytes. These cells also show differential gene expression of the myosin heavy chain (MCH) alpha and beta isoforms, expressing more of the alpha chain (90%) than the beta chain (10%). Due to localization of the human 430Q mutation on iPSc-CM in the beta chain and low levels of expression due to lack of maturation, this iPSc-CM model is not ideal to test our H10.8L shRNA. In order to test our H10.8L, we need an iPSc-CM human cell model that expresses equally the 403Q mutation and wild type allele together with the alpha isoform. In order to generate this cardiomyocyte cell system, we are inducing the expression of the beta myosin heavy chain expressing mir208-shRNA in iPS-differentiated cardiomyocytes

Methods for Design of shRNAs

Described above was the use of certain identified shRNAs. To be described here are certain methods and techniques for designing of candidate shRNAs that can be advantageously used in certain embodiments of the present invention.

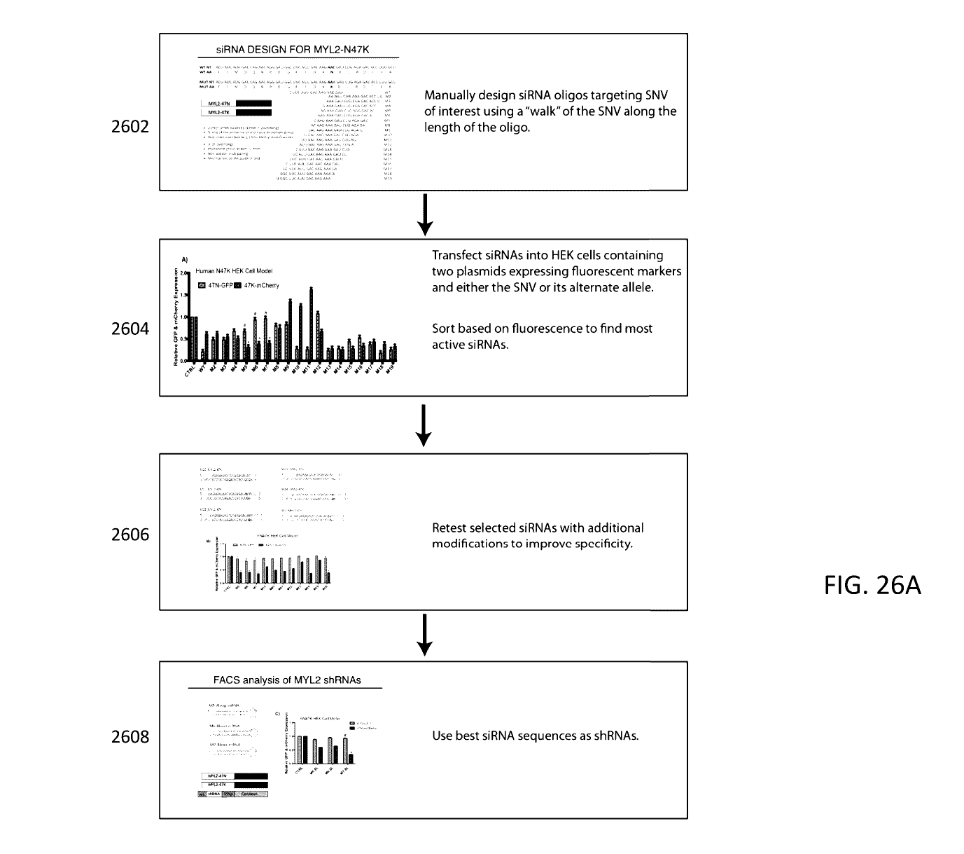

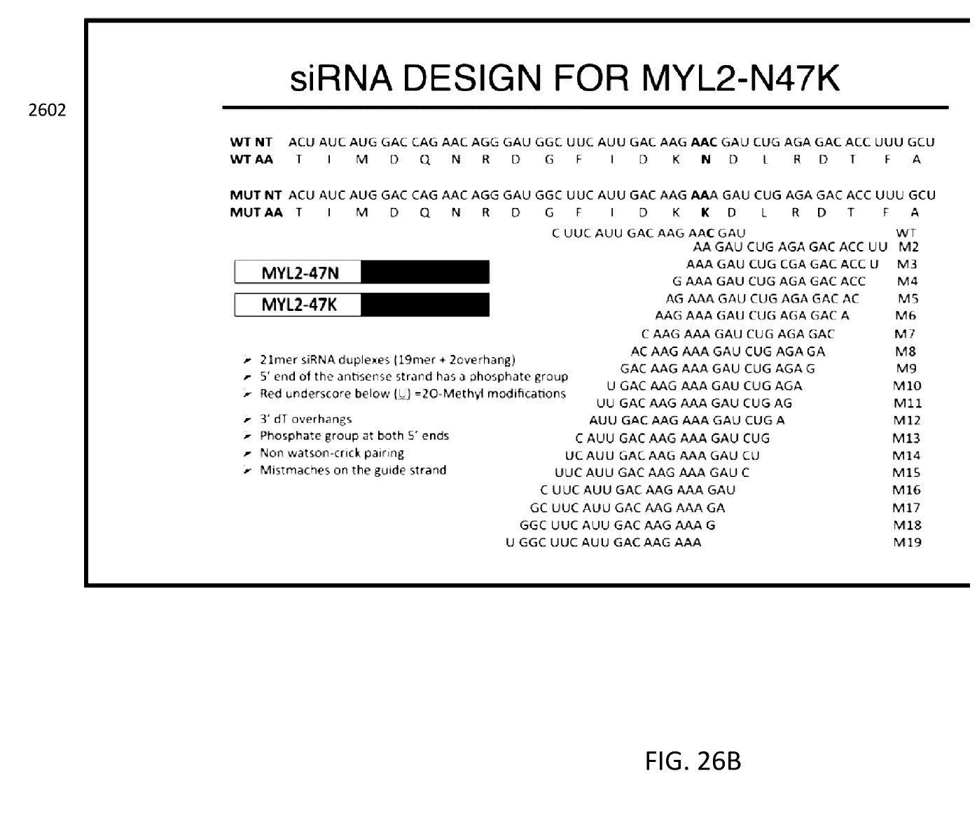

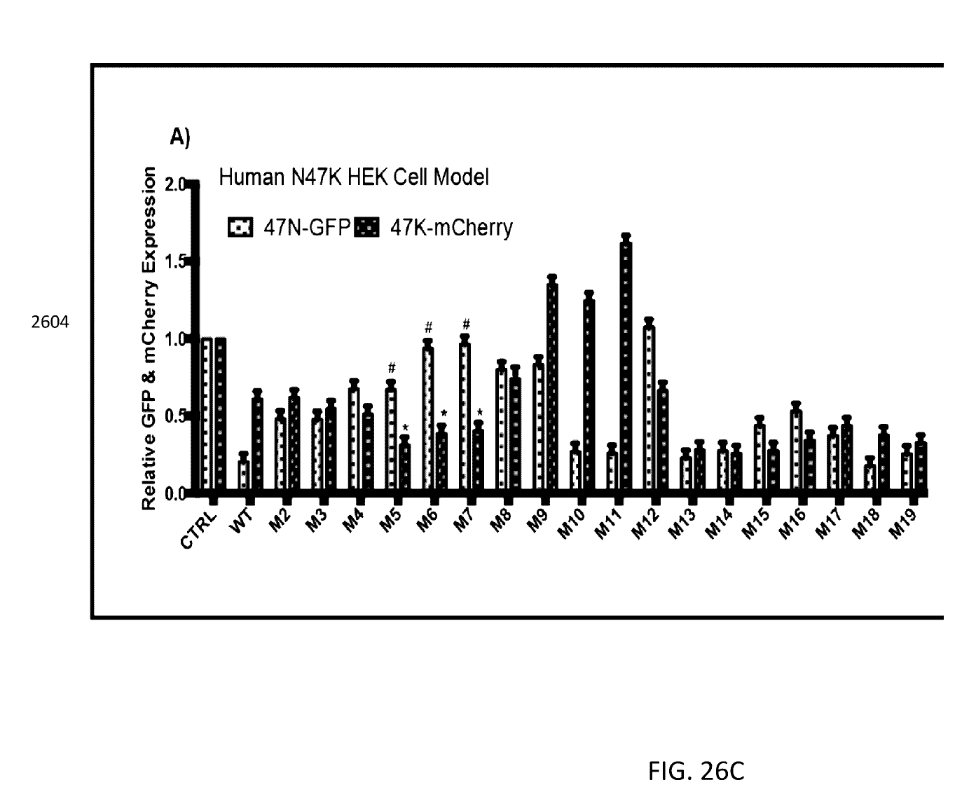

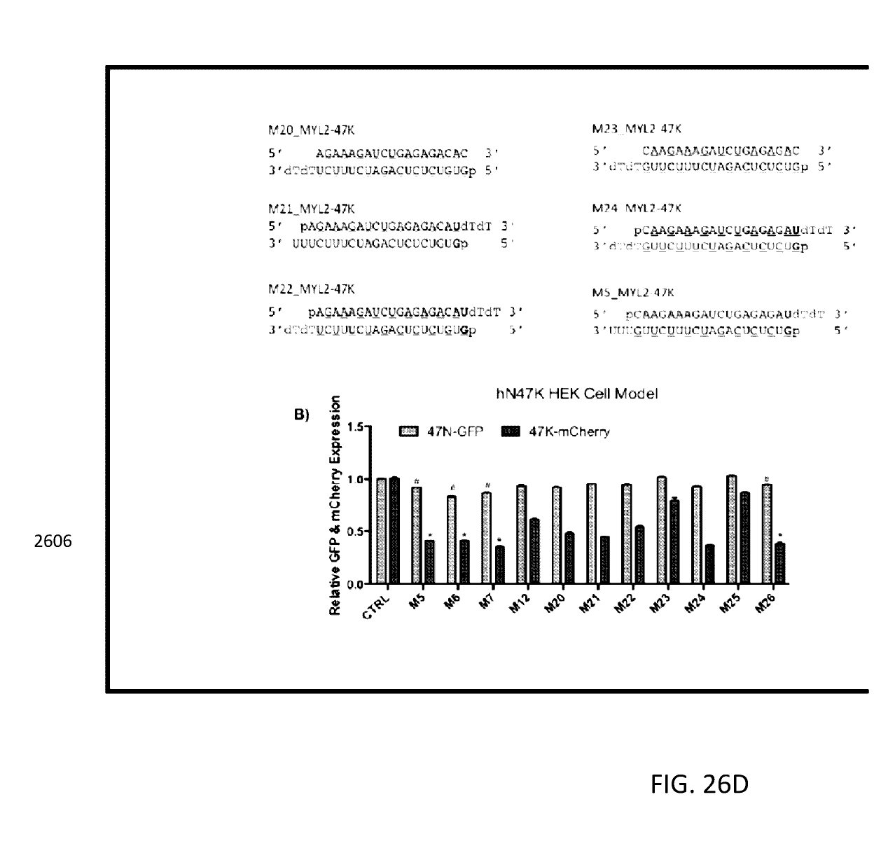

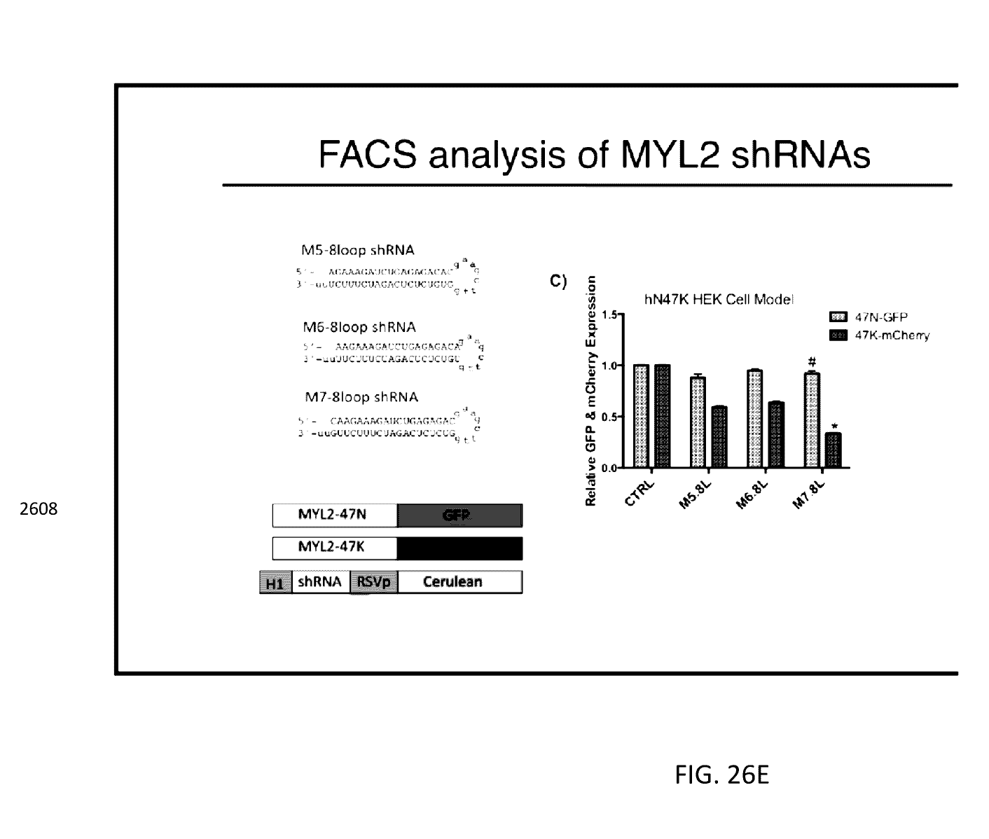

In embodiments of the present invention, it is desired to identify accurate shRNAs to target mutations of interest. FIGS. 26A-E depict a flowchart for a method for designing shRNAs according to an embodiment of the present invention. As shown in step 2602, siRNAs are individually considered for targeting an SNV of interest. In a typical situation, there are many siRNAs to consider at different positions. In an embodiment of the present invention, a length of relevant oligonucleotide is identified. From this oligonucleotide, a candidate siRNA is “walked” along the length of the nucleotide. For example, the candidate siRNA is considered at a first position along the length of the nucleotide. See for example, the blow-up of step 2602 (FIG. 26B) that shows a SNP of interest and potential siRNAs of a fixed length but at different positions along the SNP. In an embodiment, the candidate siRNA is considered at a first position, then considered at a next second position, a third position, and so on along the length of the sequence of interest.

As shown in step 2604 for an embodiment of the present invention, candidate siRNAs are transfected into HEK cells containing two plasmids expressing fluorescent markers and either of the SNV or its alternate allele. From these results a sort is performed on fluorescence in order to find certain more active siRNAs. For example, as shown in the blowup of step 2604 (FIG. 26C), silencing of the 47K-mCherry is most effective for M5, M6, and M7. In this way, they may be the best candidates for targeting the mutation. The sorting of step 2604 qualitatively organizes such information.

At step 2606, a set of candidate siRNAs are selected for retesting. For example, in an embodiment of the present invention, candidate siRNAs are retested at step 2606 with additional modifications in order to improve specificity or to make them more stable. In an embodiment of the present invention, the identified siRNAs are modified with sticky overhangs, 3′ and 5′ as shown in the blowup of step 2606 (FIG. 26D). Such sticky overhangs can provide for more stable siRNAs. For example, sticky overhangs can help with anchoring the shRNAs. Watson-Crick mismatches may also be introduced to generate an improved candidate siRNA. Retesting of the modified siRNAs may then demonstrate improvements silencing mutations of interest such as shown the blowup of step 2606. For example, as shown in the blowup of step 2606, the performance of M5 was improved when compared with M5 as shown in the blowup of step 2604.

The modifications that may be appropriate can be affected by the delivery system to be used. For example, where delivery to the heart is desired, there may not be a direct delivery system. Alternatives, can include for example viral delivery with shRNAs as described elsewhere in the present disclosure.

From the retesting, a potentially smaller set of preferred siRNA sequences are identified at step 2608. This set of preferred siRNA sequences are chosen as candidate shRNAs according to an embodiment of the present invention (see blowup of step 2608, FIG. 26E). In turn, candidate shRNAs are further evaluated for effectiveness as described separately in the present disclosure. For example, as shown in the blowup of step 2608, the shRNA attached to the Cerulean virus was used to evaluate silencing of undesirable mutations.

Because of the complexity of oligonucleotides, siRNAs, shRNA, and other genetic data, the design of shRNAs, individualized manual design is difficult in a small scale but prohibitive in large scale. Accordingly, embodiments of the present invention, include computerized methods for designing shRNAs in allele specific oligonucleotides as shown in the flowchart of FIG. 27.

In an embodiment of the present invention, a VCF (Variant Call Format) file is generated that contains all the SNPs of interest, including position and alternate/reference base information. In an embodiment, a VCF file can have 500 SNPs. At step 2702, such VCF file is input to a computer configured to implement the method of FIG. 27. Responsive to the VCF file, queries of a reference genome are performed at step 2704 for a sequence substantially surrounding the SNPs of interest. In an embodiment, the surrounding 40 base pairs are considered, thereby creating an 80 base pair box. From these queries, a set of candidate shRNAs and ASOs are created from the identified sequence surrounding the SNPs of interest as shown at step 2706.

At step 2708, a ranking is performed based on certain predetermined qualities including, binding, length, melting temperature, GC content, and other factors. In another embodiment of the present invention, a global metric is structured to perform a ranking. From a ranking, a set of candidate shRNAs are determined. Desired modifications can then be made at step 2710 to accommodate identified issues. For example, mismatches, modified backbones, loops, and other issues can be added at step 2710 as may be desired. At step 2712, a set of preferred shRNAs and ASOs are generated for each SNP of interest.

Advantageously, because of the computerized operation of certain methods of the present invention, many shRNAs, ASO, and SNPs can be investigated. Indeed, because of the volume and complexity of genetic data, certain methods of the present invention would not be possible in a pencil-and-paper operation. For example, where individualized analysis could take weeks to perform, embodiments of the methods of the present invention can yield results within minutes. The methods of FIGS. 26 and 27 are applicable more broadly than the examples described herein as would be understood by those of ordinary skill in the art. For example, embodiments of the present invention are applicable to identifying a specific reference and comparing it against known variants within disease space

### Example 2

To be described now is another experiment that addressed hypertrophic cardiomyopathy (HCM). HCM is a genetic disease of the heart muscle and the most common cause of sudden death in young people and athletes. It is caused by heterozygotic missense mutations in genes encoding proteins of the cardiac sarcomere.

Here, we present ex vivo and in vivo data for mutant allele-specific gene silencing of the N47K mutation of the regulatory light chain (RLC) according to an embodiment of the present invention. We have designed and identified an RNA interference (RNAi) construct, M7.8L, that reduced the expression of the mutated human regulatory light chain (RLC) by 45% in neonatal cardiomyocytes (NCM) and the expression of the wild type allele by 10%.

In an embodiment, Sarcomeric organization was induced with biomechanical devices to measure mechanical function of the NCM cells treated with M7.8L, which led to significant reduction in the beating rate, while sarcomeric shortening remained unchanged. In vivo studies in mutant transgenic mice showed that M7.8L reduce the expression of the mutated allele by 90%. Echocardiography studies showed that the left ventricle (LV) mass did not increase preventing the progression of the disease.

In another embodiment of the present invention, we have also identified two RNAi constructs, H10.8L and H11.8L that, in patient specific induced pluripotent stem cells cardiomyocyte (R403Q iPSc-CM), silenced the severe R403Q mutation of the myosin heavy chain gene (MYH7) in a mutant specific manner.

The outcomes of these studies provide important information for drug discovery and the development of novel genetic therapeutics for cardiovascular diseases.

Human genetic variations can lead to pathological changes in cell function and molecular mechanisms predisposing to disease. Some of these variations can be inherited and pass through generations, meanwhile others are triggered epigenetically. Currently, the availability of technologies, such as genome sequencing, precise genome editing techniques and selective gene silencing provide for gene-based therapeutics. Challenges remain including designing tools effective in cases where only a single nucleotide distinguishes a healthy gene from one that confers a severe disease phenotype. Such a dominant negative effect of a genetic mutation is the case with Hypertrophic Cardiomyopathy (HCM).

HCM is a genetic disease caused by a single nucleotide variant and is the most common inherited cardiovascular disease and is the cause of sudden death in young people and competitive athletes. It affects one person in 500, causing significant morbidity and mortality worldwide. The phenotypes of the disease include thickening of the myocardium, particularly the septum, myocyte disarray, and fibrosis. It is caused by heterozygotic missense mutations in genes encoding proteins of the cardiac sarcomere. Among these genes, cardiac myosin has been studied most extensively.

Myosin is a hexameric protein complex with two myosin heavy chains, either α-MHC encoded by MYH6 (predominant in murines) or β-MCH encoded by MYH7 (predominant in human adults) and four light chains: two regulatory light chains (RLC) encoded by MYL2 and two essential light chains (ELC) encoded by MYL3 respectively and is the molecular motor of the heart cells that generate a mechanical force by ATP hydrolysis. Single nucleotide variants (SNVs) within the catalytic domains, calcium binding domains, and phosphorylation sites of these proteins alter the mechanical forces, redox states, and cellular signals in a dominant negative manner to cause pathology.

Medical therapy for HCM remains largely palliative. Beta-blockers, calcium channel blockers, and disopyramide are the mainstay of pharmacological management. The clinical effects of these pharmacological agents are modest and often limited by side effects. In this context, gene-silencing technology by selectively reducing the expression of the mutated allele represents a novel therapeutic approach for HCM.

When it comes to studying the effect of human HCM mutations, mouse cardiomyocytes can be a poor model system since the α-MHC is the predominant isoform in murines representing a challenge to translate the experimental results to human adults where the predominant isoform is b-MHC. Additionally, in vitro motility and ATP assays have shown that alpha and beta MHC have different functional effects, which is the case for the mouse R403Q versus human R403Q. New functional models to study HCM human genetic variations, such as patient derived induced pluripotent cells-cardiomyocytes (iPSc-CM), have made it easier to track perhaps subtle phenotypes caused by genetic modifications.

Induced pluripotent stem cell derived cardiomyocytes shows promise as a good cell model to study these human genetic mutations. Human iPSc-CM, however, grows without sarcomeric organization. Phenotypically, cultured human iPSc-CM as well as murine neonatal cardiomyocytes (NCM) grows as a monolayer without sarcomeric organization, which is the immature/neonatal stage of the cells. Maturation toward an adult cardiomyocyte phenotype can be accomplished in culture with the use biomechanical devices such as microposts and micropatterning. Both techniques are important to allow the NCM and iPSC-derived cardiomyocytes to develop structural features typical of adult cardiomyocytes, thus making it possible to obtain meaningfully measurement of contractile shortening and calcium dynamics,

Here, we present our results in allele-specifically silencing the human MYL2-N47K (asparagine to lysine) and the human MYH7-R403Q (Arginine to Glutamine) mutations of the RLC and β-MHC, respectively. The human MYL2-N47K mutation interferes with Ca²⁺ binding on the RLC, affecting the rotation of the lever arm due to delayed calcium transients and thus altering the mechanical properties of the neck region producing changes in the cardiac muscle contraction and causing a severe mid-ventricular hypertrophy with a rapidly progressive phenotype.

The human MYH7-R403Q mutation is located in the globular head domain of the molecular motor of the myosin heavy chain, directly affecting its binding to actin protein. It is the most deadly mutation causing a severe phenotype due to the dominant expression of the mutated allele. R403Q mutation is the also a well-studied mutations and structural studies have showed that the mutation disrupt severely the actin-myosin interaction at the interface. The mutation causes a disruption in the Z-lines causing myocyte disarray, which is characteristic of the disease.

Here, we used a human MYL2-N47K transgenic mouse model and a human MYH7-R403Q induced pluripotent stem cells cardiomyocytes (iPSc-CMs) models and demonstrated allele specific gene silencing of both HCM mutations. We designed and used small interfering RNAs (siRNAs) and short hairpin RNAs (shRNAs) that specifically down regulated the mutated allele, delaying the progression of the disease in a human transgenic animal model.

### Materials and Methods

Small interfering RNA (siRNA) design

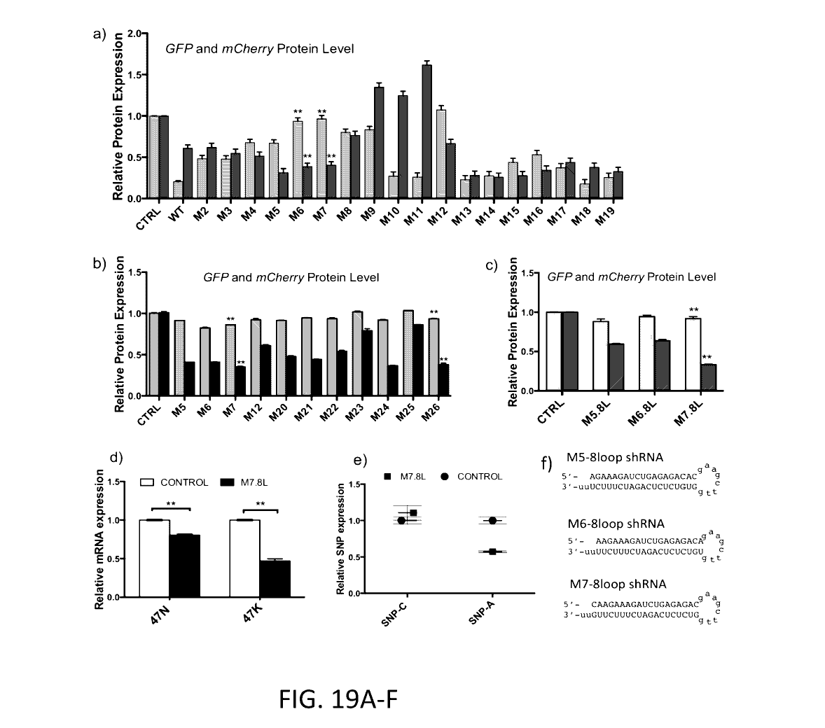

A series of 21 small interfering RNA duplex oligonucleotides were designed with the 47K mutation of the MYL2 gene in the second position of the first oligonucleotide (See FIG. 19, see also FIGS. 26 and 27). Each subsequent oligonucleotide was designed with the mutation shifted one position to the right on the native gene sequence, screening all possible targets sequences containing the mutation. A similar series of oligonucleotides were designed for the 403Q mutation of the human MYH7 gene. All were synthetized by Protein and Nucleic Acid Facility of Stanford University. The sense and antisense strands are of 19mer and 21mer, respectively, in length. Both contain alternated 2-O-Methyl modifications to increase backbone resistance against endonucleases, 2 nucleotide overhangs at the 3′ end and a phosphate group at the 5′. Mismatched pairing was also introduced.

Short Hairpin RNA (shRNA) Design

shRNA design was based on the nucleotide sequence of the sense strand of MYL2-siRNAs. In this embodiment, the 5′ end of the sense strand contains a phosphate group and the restriction site Bbs I. The antisense strand contains BbsI restriction site at the 3′ end. The complementary strand contains a C at the 3′ end and a phosphate group at the 5′ end. In this embodiment, two types of loop sequences were used: Loop 8 (CAAGCTTC) and loop 12 (CTTCCTGTCAGA). Loop 8 and loop 12 contain a Hind III and HpyCH4 III restriction site, respectively.

siRNAs and shRNAs Screening

HEK293 cells were co-transfected with plasmids coding the human RLC fused to fluorescent reporters: pMYL2-47N-GFP (wild type) and pMYL2-47K-mCherry (mutant). Lipofectamine LTX (Invitrogen) was used to mediate transfection of 100 ng of each plasmid following the manufacturer's instructions. G418 antibiotic was used at 1000 ug/ml to generate stable transfected cell lines. Flow activated cell sorting was used to isolate individual clones expressing both GFP and mCherry proteins. Sorting was performed on an LSRILUV: S10RR027431-01 instrument in the Stanford Shared FACS Facility obtained using NIH S10 Shared instrument Grant.

Similar protocol was used for the human MHC-R403Q mutation. HEK293 were co-transfected with plasmid coding the partial human sequence of MHC (880aa) attached to fluorescent reporters: pMYH7-403R-GFP and pMYH7-403Q-mCherry. Transduction and cell sorting was carried out as discussed above.

### Human Transgenic MYL2-N47K Mice

Transgenic mice were obtained from Danuta Szczesna-Cordary at the University of Miami that, in addition to the endogenous mouse MYL2, expressed either human normal MYL2-47N or human mutation MYL2-47K on a CD1 background. All animals were handled under protocols 22920 and 22922 approved by the Stanford Administrative Panel on Laboratory Animal Care (APLAC).

### Single Nucleotide Polymorphism (SNP) Analysis

Pyrosequencing was carried out on NCM and iPSc-CM for SNP analysis of the human MYL2-N47K and MYH7-R403Q respectively. For the N47K mutation the following primers were used: MYL2-Pyrosequencing Forward: ACAGGGATGGCTTCATTGACA and MYL2-Biotin-pyrosequencing Reverse: O-TTCCTCAGGGTCCGCTCCCTTA and the MYL2 sequencing primer GGCTTCATTGACAAGAA. For the R403Q mutation, the following primers were used: MYH7-pyrosequencing Forward: TATAAGCTGACAGGCGCCATCAT and MYH7-Biotin-pyrosequencing Reverse: OCCCCTTGGTGACGTACTCATTG, and MYH7 sequencing primer: GGGCTGTGCCACCCT. AmpliTaq Gold (Applied biosystems) was used for PCR amplification.

Allele Quantitative Polymerase Reaction (qPCR) Using 3′ Phosphate Specific Blockers

Total RNA was extracted (miRNeasy Qiagen) and analyzed by Agilent Bioanalyzer. cDNA synthesis (Applied Biosystems) and was carried out for allele quantitative PCR. For mutant detection and wild type discrimination, the following reaction was carried out in 20 ul volume: Mutant specific Forward (0.5 um) (18mer=GGCTTCATTGACAAGAAA) Reverse primer (0.5 um) (TTCCTCAGGGTCCGCTCCCTTA), wild-type specific blocker 1 (Sum) (TGACAAGAACGATCTGAGA-PO4), MYL2 Hidrolysis probe containing 6-FAM at the 5′ end and TAMRA at the 3′ end (5′-TGGATGAAATGATCAAGGAGGCTCCG-3′), 18S endogenous control mouse and 1× of Taqman Universal PCR Master mix, (No AmpErase UNG Part Number 4324018).

For wild type detection and mutant discrimination, the reaction was as follows: Wild type specific Forward (GGATGGCTTCATTGACAAGAAC), Reverse primer (0.5 um) (same as in the mutant detection reaction), mutant specific blocker-5 (Sum) (GGCTTCATTGACAAGAAC-PO4, Taqman probe (same as in the mutant detection reaction), 18S endogenous control and Taqman Universal PCR Master mix, (No AmpErase UNG). The qPCR reaction was performed by 95° C./10′ Hold, 95° C./30″, 50° C./1′, 60° C./1′ 35 cycles.

### In Vivo AAV9 M7.8L Transduction of MYL2-N47K Transgenic Mice

MYL2 transgenic mice at different ages were injected intrajugulary. For the old and young group, AAV9 expressing M7.8L shRNA and non-expressing shRNA (control) at concentration of 1×10¹² genomic titer. For the neonatal group, 25 ul of virus was injected. A second AAV9 luciferase construct pRSV eGFP-T2A-Fluc2 was utilized as a control to track the virus expression over time.

In Vitro AAV6 H10.8L Transduction of R403Q iPSc-CM.

Differentiated iPSC-cardiomyocytes were used which were plated in 48 well plates at a density of about 300,000 cells per well at 20 days post differentiation. Media was aspirated and replaced with either 120 ul fresh media (controls) or 100 ul fresh media plus 20 ul of AAV6-H10 virus (1.1×10⁷ IU/ml or 3.9e12 vg/ml) for a final concentration of 220,000 IU per well (7.8e10 vg/well). Cells were incubated at 37° C. 100 ul of media added every 48 hours. After six days, cells were harvested with 0.5 mM EDTA in PBS and frozen at −80° C. for RNA extraction. Total RNA was extracted with a Qiagen miRNeasy kit and analyzed by Agilent Bioanalyzer.

cDNA were synthesized using an Applied Biosystems High Capacity cDNA kit. For allele specific quantification of MYH7 R403Q, cDNA was split and digested with AvaI, which cuts at the wildtype R403R site, or Bsu36I, which cuts at the mutant R403Q site. Allele specific QPCR was then performed using mutant or wildtype specific forward primers and a common reverse primer. Each forward primer contained a mismatch at the penultimate nucleotide to increase allele specificity. R403R Forward Primer was: 5′ GGGCTGTGCCACCCTAA 3′, R403Q Forward Primer: 5′GGGCTGTGCCACCCTAG 3′, Common Reverse Primer was: 5′CGCGTCACCATCCAGTTGAAC 3′. MYH7 specific fluorescent probe were optimized for maximum sequence dissimilarity from MYH6: FAM-5′TGCCACTGGGGCACTGGCCAAGGCAGTG 3′-TAMRA. Allele specific qPCR conditions were implemented using Taqman Fast Universal PCR Master Mix: 95° C. 20″, 40 cycles of 95° C. 30″, 58° C. 20″, 72° C. 30″ for R403Q or 40 cycles of 95° C. 30″, 64° C. 20″, 72° C. 30″ for R403R. Endogenous control was 18S.

### Traction Force Microscopy of MYL2-N47K Neonatal Cardiomyocytes

Cells were cultured on 2000 μm² rectangular laminin (BD Biosciences) patterns with an aspect ration of 5:1 on polyacrylamide (PA) substrates to generate an elongated shape and sarcomeric organization in order to contract along their main axis and to present aligned sarcomere organization. Gels were fabricated as reported elsewhere. We mixed PA gel components (12% Acrylamide—0.15% N, N-methylene-bis-acrylamide) in DI water and added 50 μl of solution on clean coverslips pretreated with aminopropyltriethoxysilane and glutaraldehyde.

Polymerization occurred after we placed, on top of the gel component solution, a coverslip with stamped patterns on its surface facing the gel. We used ammonium persulfate as a catalyst for gel polymerization and N,N,N,N-tetramethylethylenediamine as an initiator. We patterned coverlips and transferred patterns to gels according to an already published method as known to those of ordinary skill in the art. We used soft lithography to fabricate polydimethylsiloxane (Sylgard) microstamps. We flooded the microstamps with 10 μg/mL laminin for 30 minutes, dried microstamps under a stream of N2 and then placed the region coated with protein on top of a pre-cleaned glass coverslip to be placed on top of the gel solution.

Once in culture, videos of contractile cardiomyocytes were acquired in brightfield with a high speed CCD camera (Orca-R2 Hamamatsu). We measured the contractile shortening of cardiomyocytes and beat rate with custom ImageJ and Matlab scripts.

### Left Ventricular Cardiomyocyte Handling.

Freshly isolated single left ventricular cardiomyocyte suspensions were first incubated in cardioplegic perfusion solution with 20 μMATP and subsequently loaded with the fluorescent ratiometric calcium dye Fura-2 acetyoxymethyl ester (AM) for Ca2+-transient measurements. After calcium was gradually re-introduced to a final concentration of 1.2 mM, the cardiomyocytes were resuspended in cardiomyocyte pacing buffer ((mmol L-1): NaCl 134, KCl 4.0, MgCl2 2, NaH2PO4 0.3, Na-HEPES 10, 2,3-butanedione monoxime 10, α-D-glucose 10, CaCl2 1.0; pH 7.4 with NaOH; 0.2 μm filtered).

### Measurement of Intracellular Calcium Transients and Contractile Function.

Intracellular Ca²⁺-transients of left ventricular, Fura-2 AM loaded, rod-shaped cardiomyocytes were recorded while simultaneously measuring the sarcomere length shortening using the IonOptix Myocyte Calcium and Contracility Recording System (Milton, Mass.). Approximately 100-150 left ventricular cardiomyocytes were loaded onto the mTCII cell chamber and suffused with 37° C. cardiomyocyte pacing buffer at a 0.5 mL/min flow-rate. The chamber was paced at 1.0 Hz and 15 Vat a duration of 5 ms.

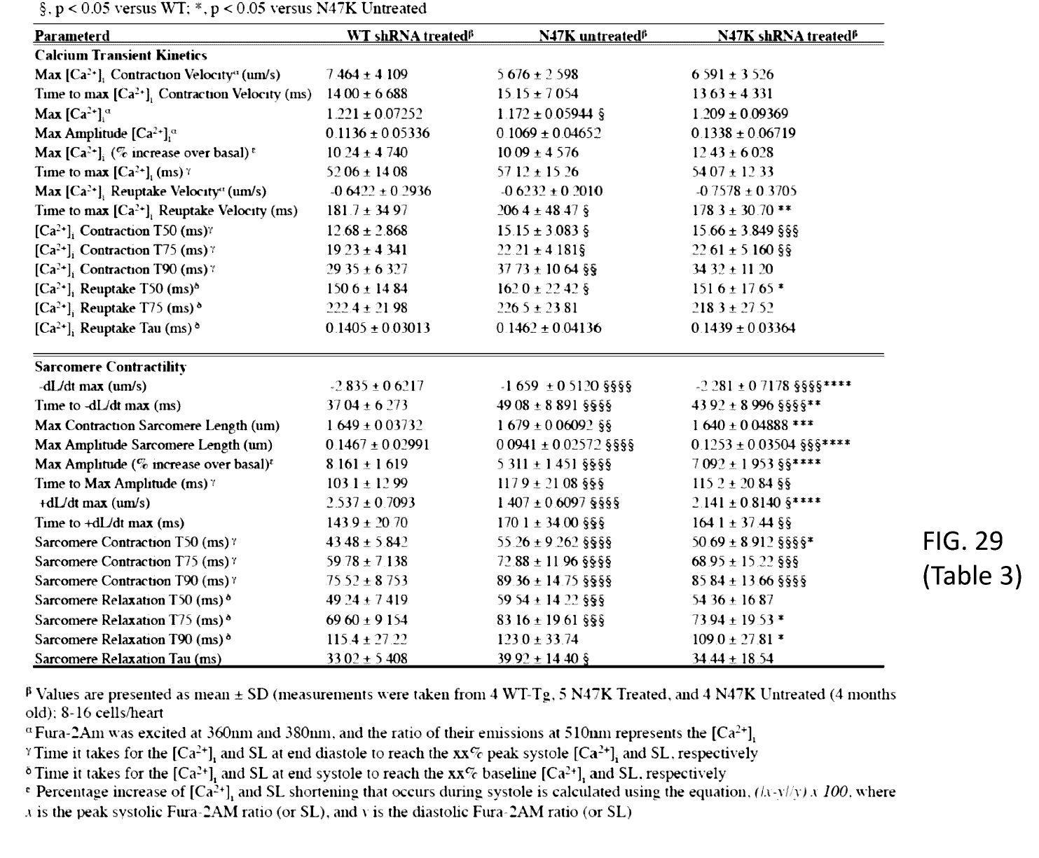

Inclusion criteria for cardiomyocyte selection consisted of completely isolated single cells with rod-shaped morphology, resting sarcomere length 1.7-1.85 μm, uniform contracility, and absence of arrhythmia. Free intracellular Ca²⁺ levels were recorded using the 340/380 nm excitation-510 nm emission ratio and velocity, time to maximal [Ca²⁺]ᵢ reuptake velocity, Ca²⁺-transient reuptake decay rate (tau), and relaxation T50. Simultaneous sarcomere shortening measurements using IonoWizard 6.0 cell dimensioning data acquisition software allow for determination of maximal velocity of sarcomere shortening (−dL/dtmax) and relaxation (+dL/dtmax), time to −dL/dtmax and +dL/dtmax, relaxation tau decay rate, and shortening and relaxation T50, 75, 90. See Table 3 shown in FIG. 29.

### Treadmill Cardiovascular Test.

Treadmill running machine channels and an O₂ sensor were calibrated until % O₂=20.94 and delay was set for 20 seconds. Groups of four mice (2-4 months old) were placed in a rodent treadmill chamber with shocks turned on and the treadmill off. The mice were allowed to acclimatize with gentle walking for 5 minutes. Treadmill exercise consisted of 21 minutes and started with an initial speed setting of 10 m/min at a flat grade, followed by speed up to 15 m/min, grade at the 5 minute mark, then at the 6 min mark an increase of speed to 17.5 m/min and grade of 10, then at the 9 minute mark a speed up to 17.5 m/min, and grade of 15; then at the 12 minute mark a speed up to 20 m/min, at a grade of 15, then at the 15 minute mark an increase of a speed to 22.5 m/min with the grade kept at 15 for the remainder of the run, then, at the 17 minute mark, a speed up to 27 m/min with the final speed of 30 m/min set at the 19 minutes point. Most mice ended their run before reaching this speed.

Stimulus grids were turned off when the RER of individual mice reached ˜1.10 or they were exhausted. Mice were left in the chambers until the end of 21 minutes. Chambers were open to air out before introducing a new batch of mice. Baseline RER should be ˜0.8.

Echocardiography was performed on mice treated with AAV9-shRNA in the neonatal period blinded to genotype and treatment group at age 4 months using VisualSonics VevoScan 2100 with cardiac package under isoflurane anesthesia at 36° C. with target heart rate 450-550 bpm. Images were acquired from standard windows. Measurements of contractile function and wall thickness were determined offline using VevoScan software. Ejection fraction (LV trace) and left ventricular mass were calculated using standard methods for each animal subject.

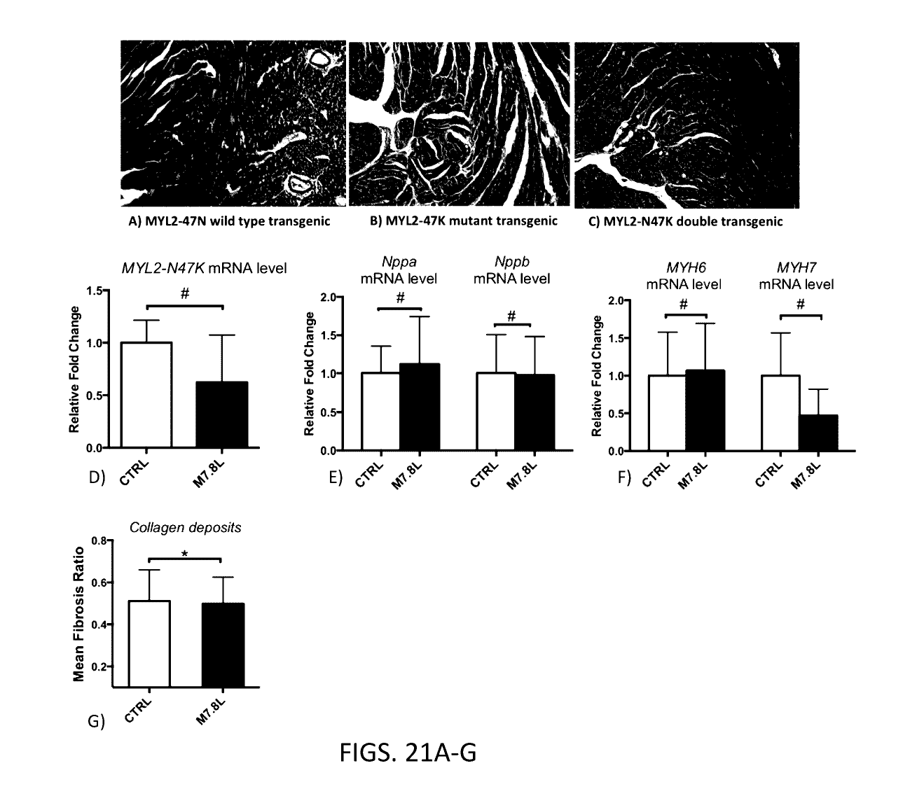

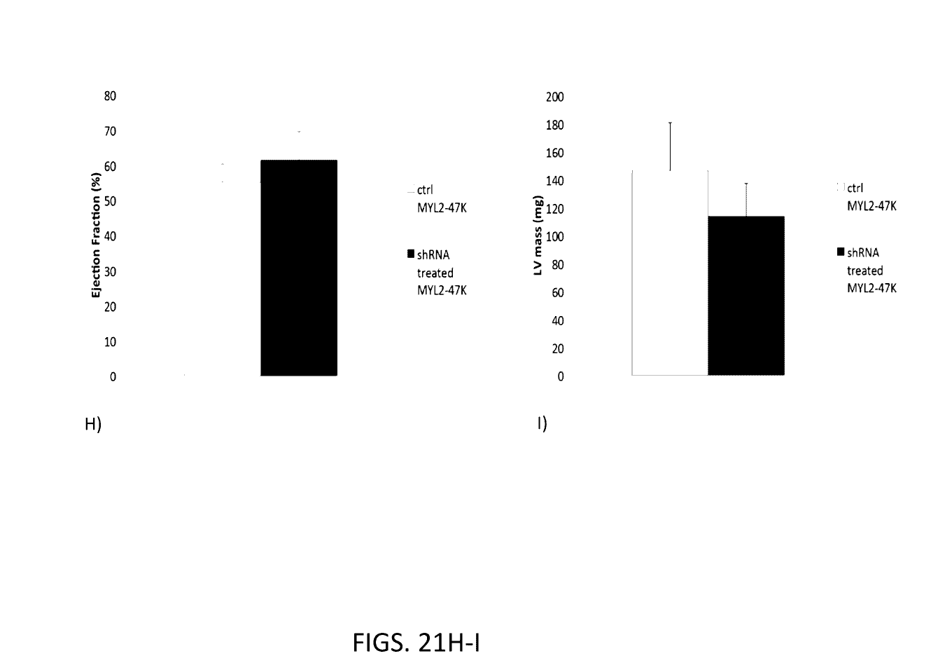

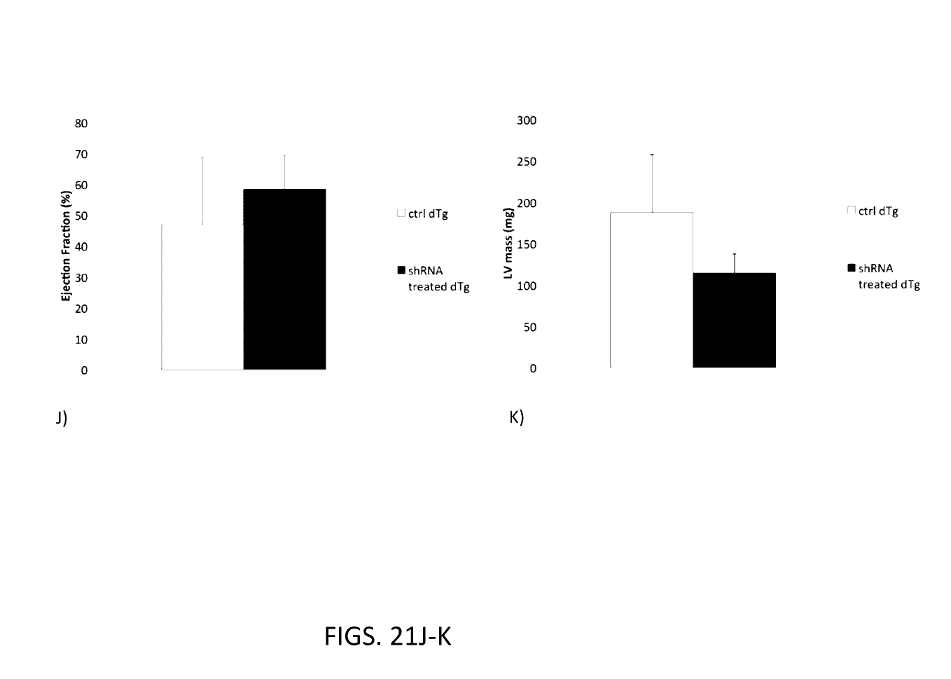

To assist in understanding embodiments of the present invention, FIGS. 21A-G show information relating to AAV9 M7.8L shRNA allele specific silenced MYL2-47K mutation in mutant transgenic mice during 4 months treatment. FIGS. 21H-I show information relating to AAV9 M7.8L shRNA allele specific silencing of MYL2-47K mutation in vivo of human mutant transgenic mouse hearts with trend toward improvement of ejection fraction (FIG. 21H) and significant reduction of left ventricular mass (FIG. 21J) (p=0.02) by echocardiography during 4 months of treatment. FIG. 21J-K. show information relating to AAV9 M7.8L shRNA allele specific silencing of MYL2-47K mutation in vivo of human double transgenic (mutant/wildtype) mouse hearts with trend toward improvement of ejection fraction (FIG. 21J) and significant reduction of left ventricular mass (FIG. 21K) (p<0.05) by echocardiography during 4 months of treatment.

### Results

Human MYL2-N47K and MYH7-R403Q Targets

Gene silencing studies were carried out in two human HCM mutations: N47K (Asp47Lys) of the regulatory light chain (RLC) encoded by the MYL2 gene, and R403Q (Arg403Gln) of the beta myosin heavy chain (β-MHC) encoded by the MYH7 gene using RNA interference molecules. To test RNA molecules, we developed a HEK293 cell model stably transfected with plasmids containing the wild type and mutant alleles fused to green (GFP) and mCherry fluorescent reporters, respectively, to explore the dynamics of position specific mismatch of small interference RNAs (siRNAs) and short hairpin RNAs (shRNAs). For MYL2-N47K silencing studies a human transgenic mouse model was used, and for the MYH7-R403Q, two patient specific induced pluripotent stem cell lines were used.

FIGS. 19A-F show information relating to position seven in siRNA and shRNA allele specific silenced MYL2-47K mutation in a HEK293 cell model stably transfected with GFP fused to the human MYL2-47N normal allele and mCherry fused to the human MYL2-47K mutated allele. FIG. 19A shows protein quantification of Green and mCherry reporters using Fluorescence activated cell sorting (FACS) after transfection with different siRNAs targeting the MYL2-N47K mutation. FIG. 19B shows protein quantification of Green and mCherry reporters using FACS after transfection with chemical modified siRNAs M5, M6 and M7. FIG. 19C shows protein level quantification of green and mCherry fluorescent reporters 62 h after transfection with plasmids expressing shRNAs M5.8L, M6.8L and M7.8L. FIG. 19D shows mRNA level quantification of the human normal and mutated alleles using quantitative PCR and specific blockers. FIG. 19E shows single nucleotide quantification of the normal ‘C’ and variant ‘A’ using pyrosequencing. CTRL=double transfected HEK cells with plasmids, MYL2-47N or normal allele fused to Green and MYL2-47K or mutant allele fused to mCherry reporters respectively. As shown, #P<0, *P<0.05, **P<0.01, ***P<0.001.

Identification of siRNAs that Allele Specific Silence MYL2-47K Mutation

Fluorescence activated cell sorting was carried out to screen nineteen siRNAs that target MYL2-47K mutation in a cell system model containing the MYL2 wild type (47N) and mutated (47K) alleles fused to green and cherry fluorescent reporters, respectively, into human embryonic kidney cells (HEK293). Control siRNA (W16) silenced 80% of the wild type allele and 40% of the mutant allele (FIG. 19). M2, M3 and M4 siRNAs silenced both alleles at the same percentage level (˜30%). M5 and M6 showed statistical significant results when they knocked down between 55-60% of the mutant allele and 20-25% of the wild type allele (see FIG. 19).

M7 siRNA showed interesting results where it silenced 55-60% of the mCherry expression and 7-10% of the green expression. M9 siRNA showed up-regulation of the mutant allele suggesting that could be used to induce HCM. M8 to M19 showed silence of both alleles from 60-80% suggesting that point mutation starting at the nucleotide position 10 is not either mutant or wild type specific.

To increase specificity and efficacy of M5, M6 and M7 siRNAs, chemical modifications such as non-pair Watson crick pairing in the guide strand, dT overhangs at the 3′ end and phosphate groups in the 5′ ends were made on these three siRNAs. siRNAs containing additional mismatches also didn't improve their efficacy and specificity compared to the original siRNA.

Identification of M7.8L shRNA that Silence Human RLC-47K Mutation

An shRNA library was prepared in a double strand adeno-associated (AAV) viral vector to target human mutation MYL2-47K. In parallel, HEK293 cells were stably co-transfected using Lipofectamine and sorted to express equally wild type and mutant alleles attached to green and mCherry fluorescent proteins, respectively.

The anti-MYL2-N47K shRNA library was screened looking at reduced expression of reporter fluorophores analyzed by Flow Activated Cell Sorting (FACS). The screen identified three shRNAs M5.8L, M6.8L and M7.8L with high efficacy and specificity in targeting MYL2-47K mutation without affecting the wild type allele (MYL2-47N) (FIG. 19). During experiments, M7.8L shRNA showed more consistency, silencing 65% of the MYL2-47K mutation without affecting the expression of the wild type allele (MYL2-47N). M7.8L shRNA showed the greatest consistency, silencing 65% of the MYL2-47K mutation but only 10% of the wild type allele.

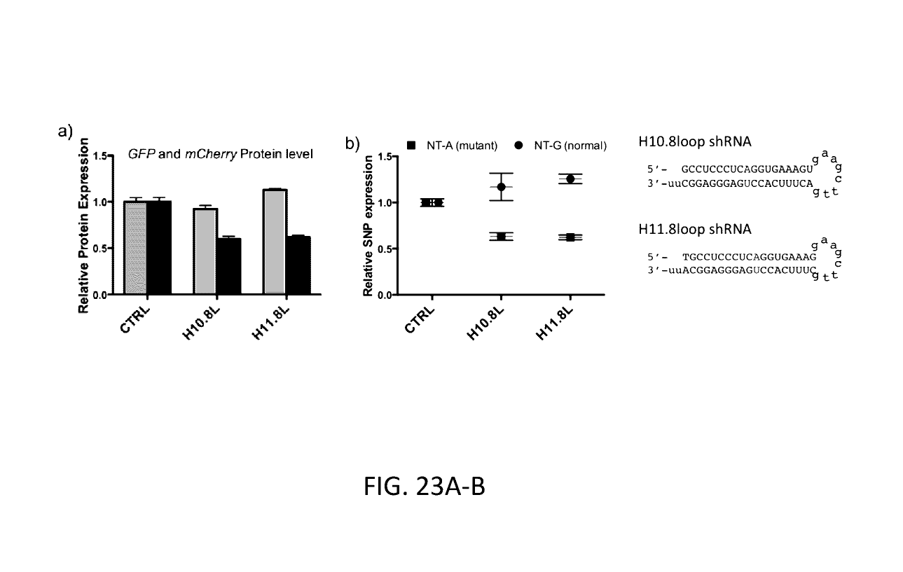

FIG. 23A-B shows information relating to H10.8L and H11.8L shRNA silenced MYHY-403Q mutation. As shown, UT=Untreated; Ctrl=mice treated with AAV9 non-expressing shRNA; M7.8L=mice treated with M7.8L RNAi. #P<0, *P<0.05, **P<0.01, ***P<0.001.

Identification of H10.8L and H11.8L shRNAs that Allele Specific Silenced Human MHC-R403Q Mutation.

An shRNA library in AAV viral vector was prepared to target the human mutation MHC-R403Q. Studies in HEK293 cells double transfected with wild type and mutant alleles fused to green and mCherry fluorescent reporters, respectively. To identify the possible hits, flow cytometry studies were carried out. We found that two shRNAs, H10.8L and H11.8L, showed similar allele silencing. Both decrease the expression of the mutant allele by 40%. H10.8L decreased the expression of the wild type allele by 10%, while H11.8L increased the expression by 10%.

### M7.8L RNAi Silenced the RLC-N47K Mutation in Double Transgenic NCM

Neonatal cardiomyocytes (NCM) were isolated from hearts of three-day old transgenic mice expressing human wild type (RLC-47N) and mutant (RLC-47K) regulatory light chain (RLC). The mice were genotyped for identification. Approximately 250,000 NCM cells per well were cultured in 48 well plates and transduced after 24 hours of cell culture with AAV9 expressing M7.8L shRNA and the control virus (not expressing shRNA) with an infectious titer of 2×10⁶. Cells were incubated for 96 hours and collected for total RNA extraction, cDNA synthesis, and quantitative PCR.

Data showed that M7.8L silenced the mutant allele by 40%, affecting the wild type allele by 10%. To assess cell function after treatment with M7.8L, NCM were cultured in micropatterning devices to induce sarcomeric organization and measure contractile shortenings, which led to significant reduction in the beating rate while sarcomeric shortening remained unchanged.

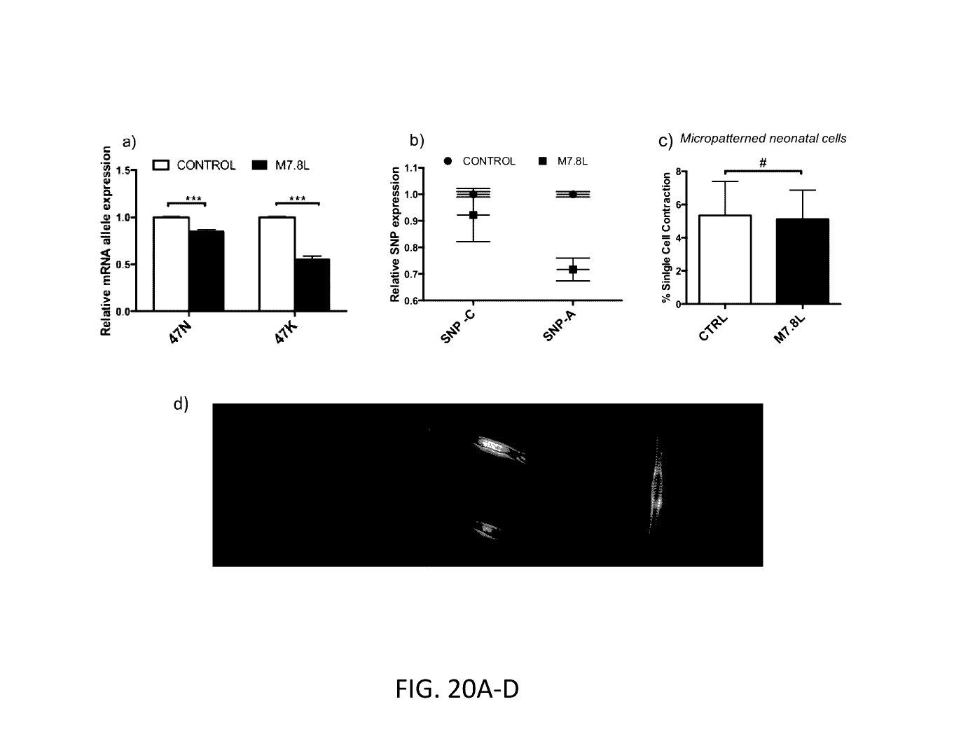

FIGS. 20A-D show information relating to M7.8L shRNA allele specific silenced MYL2-47K mutation in Neonatal human double transgenic cardiomyocytes. FIG. 20A shows mRNA level quantification of the human normal and mutated alleles using quantitative PCR and specific blockers 4d after transduction with AAV9 expressing M7.8L shRNA and Cerulean reporter. FIG. 20B shows single nucleotide quantification of the normal ‘C’ and variant ‘A’ using pyrosequencing 4d after transduction with AAV9 expressing M7.8L shRNA and Cerulean reporter. FIG. 20C shows contraction percentage of single neonatal cardiomyocytes subjected to micropatterning and transduced with AAV9 expressing M7.8L shRNA. FIG. 20D shows at left: Mouse MYL2-N47K transgenic neonatal cardiomyocytes transduced with AAV9 expressing M7.8L shRNA and cerulean reporter, middle: Mouse MYL2-N47K transgenic neonatal cardiomyocytes cultured in micropatterning wells, and at right: Neonatal cardiomyocyte in microppatterning wells.

FIGS. 21A-G show information relating to AAV9 M7.8L shRNA allele specific silenced MYL2-47K mutation in mutant transgenic mice during 4 months treatment. FIGS. 21H-I show information relating to AAV9 M7.8L shRNA allele specific silencing of MYL2-47K mutation in vivo of human mutant transgenic mouse hearts with trend toward improvement of ejection fraction (FIG. 21H) and significant reduction of left ventricular mass (FIG. 21J) (p=0.02) by echocardiography during 4 months of treatment. FIG. 21J-K. show information relating to AAV9 M7.8L shRNA allele specific silencing of MYL2-47K mutation in vivo of human double transgenic (mutant/wildtype) mouse hearts with trend toward improvement of ejection fraction (FIG. 21J) and significant reduction of left ventricular mass (FIG. 21K) (p<0.05) by echocardiography during 4 months of treatment.

### Effect of Oligonucleotide Therapy on Sarcomere Contractility Kinetics.

Sarcomere contractility studies of untreated adult MYL2-47N (wild type) and MYL2-47K (mutant) transgenic mice were initially assessed in externally paced single left ventricular cardiomyocytes. The MYL2-47K transgenic mice showed drastic dysfunction in all measured aspects of cardiomyocyte contractile functioning as compared to the age-matched MYL2-47N transgenic mice. The 47K mice had a markedly reduced maximal contraction velocity as compared to wild-type transgenic mice (P<0.0001) (see FIG. 21), which was significantly rescued by treatment with the oligonucleotide therapeutic (P<0.0001).

The untreated N47K mice also had a severely prolonged contractile phase with an elevated time to all evaluated time-points including the time to 50% maximal contraction T50 (P<0.0001) and the time to maximal amplitude (P=0.0004) (see FIG. 21). Treatment of the N47K mice yielded a significant improvement in the early phase of sarcomere shortening with a decrease in both the T50 (P=0.0103) and the time to maximal contraction velocity (P=0.0032) (see FIG. 21. relaxation kinetics. N47K mice had a marked reduction in the maximal relaxation velocity (P<0.0001), which was significantly restored in the oligonucleotide treated N47K mice (P<0.0001) (see FIG. 21. As with the contraction kinetics, the untreated N47K mice also showed a significant prolongation in the relaxation phase with an elevated time to all recorded time-points within the sarcomere recovery phase (Table 3, FIG. 29) including the T50 (P=0.0007) and the Tau decay rate (P=0.0459) (see FIG. 21). Oligonucleotide treated N47K mice showed a trend toward improvement for the early relaxation phase time-points with a statistically significant decrease in the late phase relaxation time points T75 (P=0.0101) and T90 (P=0.0450).

### Effect of Oligonucleotide Therapy on Cardiomyocyte Calcium Transient Reuptake.

Calcium transient recordings were simultaneously obtained with cardiomyocyte sarcomere shortening measurements. No significant differences in the maximal [Ca²⁺], reuptake velocity or Tau decay rate were observed between the wild-type transgenic, untreated N47K mutant transgenic, or the oligonucleotide treated N47K transgenic mice. Although the rate of [Ca²⁺], reuptake was not significantly perturbed by the N47K mutation, the T50 (P=0.0386) and time to maximal reuptake velocity (P=0.0246), both in the early phase of [Ca²⁺], reuptake, were prolonged in the N47K mutant transgenic mice (see FIG. 21). Treatment of the N47K mice yielded a complete recovery in the T50 (P=0.0289) and time to maximal reuptake velocity (P=0.0021) (see FIG. 21). See also Table 3 shown in FIG. 29.

### Discussion

Gene silencing studies were carried out on two human HCM mutations: N47K (Asp47Lys) of the regulatory light chain (RLC) encoded by the MYL2 gene and R403Q (Arg403Gln) of the beta myosin heavy chain (b-MHC) encoded by the MYH7 gene using RNA interference molecules.

The regulatory light chain RLC structurally supports the alpha helical lever arm of the myosin heavy chain MHC, which is a critical region for proper mechanical function. Also functioning as a modulatory element, the RLC is essential for force transmission and myosin strain sensitivity. Importantly, the regulatory light chain contains an EF-hand Ca²⁺-Mg²⁺ binding site in the N-terminal domain, which has structural consequences dependent on the presence or absence of bound divalent cation.

The RLC-N47K mutation completely disrupts Ca²⁺ binding to the RLC, causing an irregular conformational change of the entire head of the myosin heavy chain and contractile force and the intracellular calcium uptake. In addition to the N47K mutation, which has been shown to cause delayed onset rapidly progressing mid-ventricular hypertrophy, several other mutations within the N-terminal region also disrupt Ca²⁺ binding to the RLC (E22K, R58Q, D166V) and present clinically with varying degrees of pathologic cardiac hypertrophy. Reconstituted cardiac myofilament and cellular studies with recombinant human ventricular N47K RLC have shown that the principle defects by which the N47K mutant RLC engenders its pathologic cardiac dysfunction is via a reduction in isometric force, power output, and load at which peak power is achieved. Specifically, these alterations in actomyosin biochemistry have been shown to be related to the mutation-induced disruption of the mechanical properties of the myosin neck region, leading to a reduction in myosin strain sensitivity of ADP affinity.

The MHC-R403Q mutation occurs at the base of the loop of the b-myosin heavy chain that binds to actin, affecting the myosin-actin binding and impairing ATPase activity causing severe HCM.

To test RNA molecules, we developed a HEK293 cell model stably transfected with plasmids containing the wild type and mutant alleles fused to green (GFP) and mCherry fluorescent reporters respectively to explore the dynamic of position specific mismatch of small interference RNAs (siRNAs) and short hairpin RNAs (shRNAs). Our studies also include a murine animal model for the RLC-N47K mutation and patient specific-iPSc-cardiomyocytes for MHC-R403Q.

siRNAs and shRNAs were designed to allele specific target MYL2-47K and MHC-403Q HCM mutations. In an embodiment, siRNAs were 21mer duplexes and, although cellular kinases rapidly phosphorylate 5′OH ends and was not necessary to phosphorylate synthetic siRNAs, a phosphate group was added at the 5′ end of the antisense strand. siRNAs also have alternating 2′-O-methyl (2′OMe) residues, which provide significant nuclease stabilization to evade degradation. These modifications also prevent activation of IFN response. With these standard modifications, fluorescence activated cell sorting was used to screen siRNAs that target mutations MYL2-47K and MYH7-403Q. M5, M6 and M7 siRNAs decreased the expression of 47K-mCherry from 50-65% and the 47N (wild type)-EGFP by 10% while H10 siRNA decreased human MYH7-403Q (mut)-mCherry expression by 70% and the wild type-EGFP by 10%.

To increase specificity and efficacy of the small RNA molecules, chemical modifications were made on M5 (M20, M21 and M22) and M7 (M23, M24 and M5) to increase efficacy and specificity. Sticky overhangs at the 3′ end; such as deoxythymidines (3′ dT) nucleotide overhangs and uridine residues, were added to M5 and M7 to allow effective formation of the siRNAs with the liposome-based reagent and enhance cellular uptake. We also incorporate G-U non-pair Watson-Crick pair into siRNAs stems to avoid disruption of the helical structure. We also added these modifications near the single nucleotide variant. Our results showed that 3′dT overhangs increased the expression of the wild type allele and specificity for silencing the mutant allele, but changes in the fluorescent reporters were not significant compared to the original M5 and M7 siRNAs in this embodiment. Meanwhile, a non-pair Watson-Crick base pairing near to the variant silenced neither wild type nor mutant alleles. The combination of G-U pair in the siRNAs stem and 3′dT overhangs showed significant specificity and efficacy on the expression of the mutant and wild type allele.

Since stable long-term transfection is an ultimate goal, shRNAs analogous to best performing siRNAs were designed as 50mer length with phosphate group and a Bbs I restriction site at the 5′ of the sense strand, a Hind III loop (loop 8=CAAGCTTC). Sense and antisense strands were annealed and cloned in a self-complementary adeno-associate (scAAV) plasmid vector in the BbsI restriction sites. shRNA cloning was confirmed by Hind III/Nhe I double digestion and sequencing. Fluorescent activated cell sorting showed that M5.8L, M6.8L and M7.8L shRNAs decreased the RLC-47K (mut)-mCherry expression from 50-65% and the RLC-47N (wild type)-EGFP by 10%, while H10.8L and H11.8L shRNA decreased human MYH7-403Q (mut)-mCherry expression by 65% and the wild type-EGFP by 25%.

Pyrosequencing analysis of the RLC-N47K showed the same results as FACS but not for MHC-R403Q. SNP analysis showed that H10.8L and H11.8L shRNAs decreased “A” SNP by 40% and increases the expression of the “G” SNP by 12%. This may be due to the “GGG” polymer region in the targeting mutation. mRNA transcripts were also measured by quantitative PCR using primer specific blockers for allele discrimination, M7.8L shRNA reduced mRNA transcripts for the N47K mutant allele by 50% while the wild type allele was reduced by 10%, while H10.8L and H11.8L showed same expression analysis as observed in FACS analysis. After identifying and selecting shRNAs M7.8L and H10.8L and H11.8L that allele specific silence N47K and R403Q mutations, respectively, we made self-complementary AAV virus serotype 9 of these three shRNAs for transduction of transgenic mouse neonatal cardiomyocytes and R403Q iPSc-CM, respectively.

Ex-vivo gene silencing studies of the MYL2-N47K mutation were carried out in neonatal cardiomyocytes isolated from three-day old MYL2-N47K transgenic mice containing both human alleles: MYL2-47N (wild type) and MYL2-47K (mutant). The mice containing both human alleles are identified by PCR that discriminate the mouse endogenous RLC and Bgl II digestion. Bgl II cuts the mutant allele yielding two bands and does not cut the wild type allele, leaving the PCR product intact. Therefore, a mouse containing both human alleles yields three DNA bands. NCM from each neonatal mouse were cultured in 4 wells from a 48-well plate and transduced with 1×10⁶ infectious titer of AAV9 expressing M7.8L shRNA and incubated during 4 days.

NCM showed high transduction efficiency and 80% of cerulean reporter expression (see FIG. 20). Allele specific quantitative PCR and pyrosequencing showed that MYL2-47K mutated allele decreased by ˜50% following treatment with M7.8L and the MYL2-47N normal allele decreased 15% (FIG. 20). Transduced NCM were also subjected to growth under micro patterning conditions to explore the mechanical effects and the role of force generation of the cells with a modest decrease in contractile shortening seen.

Pre-clinical animal studies were carried out in three different groups of MYL2 transgenic mice: i) old group (AAV9 M7.8L shRNA treatment started at 7 months old), ii) young group (treatment started at 2 months old), and iii) neonatal group (treatment started at 3 days old). Each group has three different genotypes: RLC-47N (human wild type transgenic (wtTg)), RLC-47K (human mutant transgenic (mutTg)) and RLC-N47K (contains both alleles, human wild type and human mutant transgenes/double transgenic (dTg)). Before and after oligonucleotide treatment, each group was subjected to treadmill exercise to measure maximal oxygen uptake (VO2 max), echocardiography for heart function and single cells studies to measure contractile shortenings and calcium dynamics. Double (dTg) and mutant transgenic (mutTg) mice from the old transgenic group was challenging due to the high increase of deaths causing disproportion in the groups for comparison. Both genotypes from this group showed significantly low ejection fraction (EF) and impaired exercise tolerance compared to the wild type transgenic (wtTg) mice before treatment suggesting that the disease was already extremely advanced at the time of therapeutic treatment and the probability for reversion of disease was unlikely. Single cell ionOptix studies were also challenging for the mutTg and dTg mice. Hearts for both of these genotypes were significantly larger than normal hearts, making the heart cardiomyocyte isolation difficult.

Additionally, the severity of cardiac dysfunction limited our ability to perform cellular contractility and calcium transient analyses. Existing laboratory cardiomyocyte isolation techniques were modified to permit a higher yield of rod-shaped cardiomyocytes from cardiac digestion. Using a langendorf perfusion strategy for retrograde digestion of the myocardium through the coronary arteris, the flow pressure was maintained above 40 mmHg to ensure adequate perfusion through the microvascular of the inner myocardium. Presumably, due to global cardiac fibrosis and alterations in the microvasculature of the myocardium, the cell yields were improved by increasing the enzymatic digestion time while adjusting the collagenase concentration to 300 U/mL. Despite the effectiveness of these technical manipulations, enhancing the quality and yield of cardiomyocytes in the younger transgenic and neonatal cohorts, the cardiomyocytes of the old transgenic group remained intolerant to cell isolation and no single-cell functional studies were achieved. As a consequence of these difficulties, we next evaluated the effect of treatment at an earlier time in disease progression.

The young transgenic group, where treatment started at two months of age and ended four months later, showed a similar trend as the double transgenic mice from the old group. The dTg mice treated at 2 months old also showed an increase of death and intolerance to exercise. This trend was not the same for the mutant transgenic (mutTg), where M7.8L RNAi reduces the expression of the mutant allele 52% and the normal allele by 29%. These findings were consistent with echocardiographic studies cell studies that showed that heart function of the mutTg only prevented the progress of the left ventricular mass.

Because HCM phenotype in human adults is also characterized by cardiac fibrosis, collagen deposits were quantified by trichrome staining in all transgenic mice. Consistent with the echocardigrapy studies, cardiac fibrosis did not increase in the mutant and double transgenic mice compared with the control mice. Unexpectedly, natriuretic peptides (NPs) such as atrial natriuretic peptide (ANP) and brain natriuretic peptide (BNP) decreased their expression by 40% and 30%, respectively, after treatment with M7.8L shRNA in muTg but increase their expression by four folds and two folds, respectively, in the dTg when treated at two months old. Hypertrophic marker, MYH7, was also quantified, in mutTg decrease by 70% and in dTg increase seven folds. MYH6 expression remained unchanged in both, mutTg and dTg.

The neonatal transgenic group, where treatment started at three days old, showed significant specific silencing of the mutant allele by more than 90% in mutTg mice. To validate this result, the virus expression was quantified in the heart samples using cerulean reporter expression. Quantitative PCR of cerulean expression showed 20-22 cycles of abundance, meanwhile untreated mice was undetermined. Expression of hypertrophic markers ANP and BNP were also significantly reduced by 90% and 70%, respectively. MYH7 was also significantly reduced by 90%. These findings suggest that early treatment with M7.8L RNAi can prevent HCM.

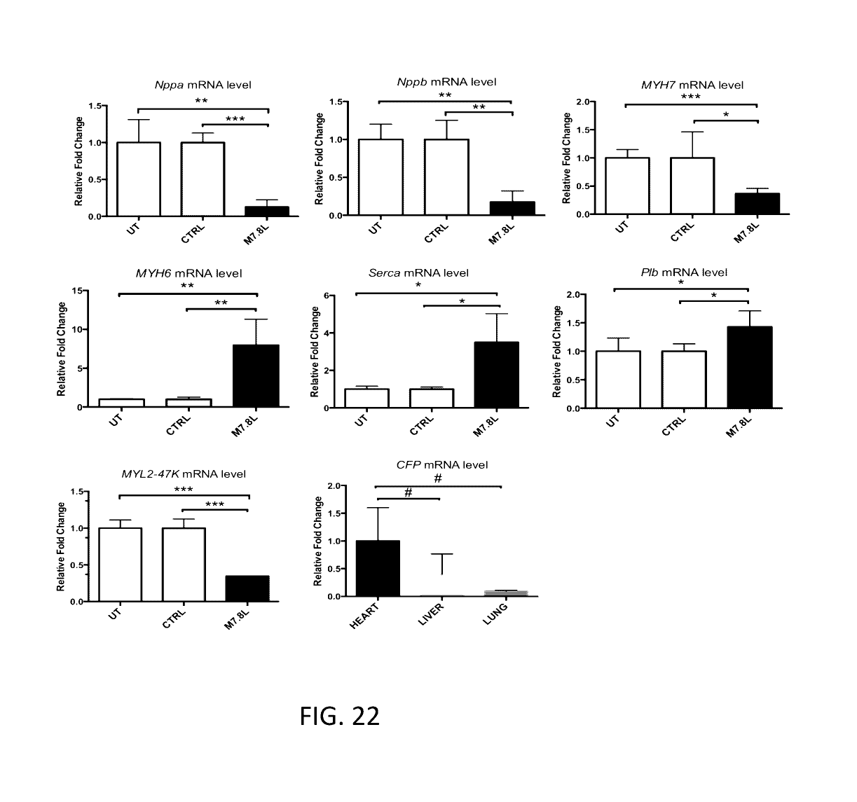

FIG. 22 shows information relating to M7.8L shRNA silenced MYL2-47K mutation in vivo and decreased the expression of hypertrophic biomarkers. Among other things, shown are mRNA levels of hypertrophic biomarkers and calcium regulators in MYL2 human mutant transgenic (mutTg) mice at 4 months of age and treated at 3 days old with M7.8L RNAi. UT=Untreated; Ctrl=mice treated with AAV9 non-expressing shRNA; M7.8L=mice treated with M7.8L RNAi. #P<0, *P<0.05, **P<0.01, ***P<0.001.

Off-Target Effects in the Lungs and the Liver were Also Assessed after M7.8L Treatment.

### Oligonucleotide Therapy Restores Cardiomyocyte Contractility and Calcium Handling.

Consistent with these studies, we observed that the N47K mutation induces a compensatory hypertrophy by impairing both cardiomyocyte contraction and relaxation as well as causing abnormal intracellular calcium handling.

The observed reduction in maximal contraction velocity and prolonged contraction phase may be a direct result of disrupted RLC Ca²⁺ binding, consequently impairing transmission of strain to the active site of the MHC. Here, we showed that in vivo treatment of neonatal N47K mutant transgenic mice with allele specific oligonucleotide therapy targeted at the N47K mutant allele resulted in a significant restoration in the maximal contraction velocity and early phase of sarcomere shortening as compared to the wild-type transgenic mice.

Untreated N47K mutant transgenic mice also displayed pathologic perturbations in the relaxation kinetics of cardiomyocyte contractility with a marked reduction in the rate of relaxation. This elongation of the sarcomere recovery phase was also accompanied with a significant prolongation in [Ca²⁺]ᵢ reuptake. This suggests that, in addition to its primary role in mechanical disruption of sarcomere contractility, the N47K mutation also secondarily induces alterations in [Ca²⁺]ᵢ homeostasis. The specific mechanism by which the mutation engenders abnormalities in [Ca²⁺]ᵢ reuptake kinetics was not investigated here, but may result from a disruption in the RLC's role as a molecular regulator of myofilament intracellular calcium handling, resulting in prolonged [Ca²⁺]ᵢ transients. This may become functionally important at higher beat frequencies as the [Ca²⁺]ᵢ transients begin to fuse, preventing complete relaxation and diastolic dysfunction. Treatment of the N47K transgenic mice yielded a drastic improvement in the maximal rate of sarcomere relaxation with modest improvements in the time to recovery. Treatment also completely restored cardiomyocyte [Ca²⁺]ᵢ reuptake as compared to wild-type transgenic mice.

Gene silencing of the MYH7-R403Q mutation was carried out in patient-specific iPS cardiomyocytes. R403Q iPSc were initially expanded and differentiated to cardiomyocytes using CHIR99021 and IWR-1 together with B27 minus insulin and transduced with AAV9 virus expressing H10.8L and H11.8L shRNAs and a combination of both. We found that two critical factors were affecting the allele specific silencing of the R403Q mutation: i) B27 minus insulin complement contains T3 (triodo-I-thyronine), a thyroid hormone that repress the expression of beta-MHC and ii) AAV virus serotype 9 was ineffective to transduce iPSc-CM. Our current cardiac differentiation utilized recombinant human albumin and L-ascorbic acid 2-phosphate and AAV6 expressing H10.8L and H11.8L shRNAs, which have successfully silence the human R403Q mutation.

### Example 3

To be discussed now is another experiment relating to induced pluripotent stem cells (iPSCs) that can be an important model system. When derived from patients, iPSCs provide a genetically matched system for drug development and discovery. Using previously described iPSC to cardiomyocyte differentiation protocols, we have used iPSC-derived cardiomyocytes to show that our gene silencing techniques can prove to be effective at silencing a mutant allele in a human model of hypertrophic cardiomyopathy. We attempted to silence a mutant allele of MYH7 using virally delivered shRNAs.

Silencing the R403Q Mutation Using Virally Delivered shRNAs

shRNAs targeting the R403Q mutation in MYH7 were designed and packaged into AAV6 viral vectors. The targeting shRNA sequence included sense and antisense targeting sequences separated by a HindIII restriction site: 5′-caccgGCCTCCCTCAGGTGAAAGTgaagcttgACTTTCACCTGAGGGAGGCtttt-3′. Differentiated iPSC-cardiomyocytes heterozygous for the R403Q mutation were plated in 48 well plates at a density of about 300,000 cells per well.

In trial one, day 40 cardiomyocytes were used. Media was aspirated and replaced with either fresh media (controls) or fresh media with AAV6-H10 virus at a final concentration 7.8e10 vg/well. Cells were incubated at 37 C. After six days, cells were harvested with 0.5 mM EDTA in PBS and frozen at −80 C for RNA extraction.

In trial two, day 16-18 cardiomyocytes were used. Media was aspirated and replaced with either fresh media (controls) or fresh media with AAV6-H10 virus at a final concentration 3.9e10 vg/well. Cells were incubated at 37 C. After four days, cells were harvested with 0.5 mM EDTA in PBS and frozen at −80 C for RNA extraction.

Total RNA was extracted with Qiagen miRNeasy kit and analyzed by Agilent Bioanalyzer. cDNA was synthesized using Applied Biosystems High Capacity cDNA kit.

For allele specific quantification of MYH7 R403Q, cDNA was split and digested with AvaI, which cuts at the wild type R403R site, or Bsu36I, which cuts at the mutant R403Q site. Allele specific QPCR was then performed using mutant or wild type specific forward primers and a common reverse primer. Each forward primer contained a mismatch at the penultimate nucleotide to increase allele specificity. R403R Forward Primer: 5′ GGGCTGTGCCACCCTAA 3′, R403Q Forward Primer: 5′GGGCTGTGCCACCCTAG 3′, Common Reverse Primer: 5′CGCGTCACCATCCAGTTGAAC 3′. MYH7 specific fluorescent probe optimized for maximum sequence dissimilarity from MYH6: FAM-5′TGCCACTGGGGCACTGGCCAAGGCAGTG 3′-TAMRA

Allele specific qPCR conditions using Taqman Fast Universal PCR Master Mix: 95 C 20″, 40 cycles of 95 C 30″, 58 C 20″, 72 C 30″ for R403Q or 40 cycles of 95 C 30″, 64 C 20″, 72 C 30″ for R403R. Endogenous control is 18S.

### Results

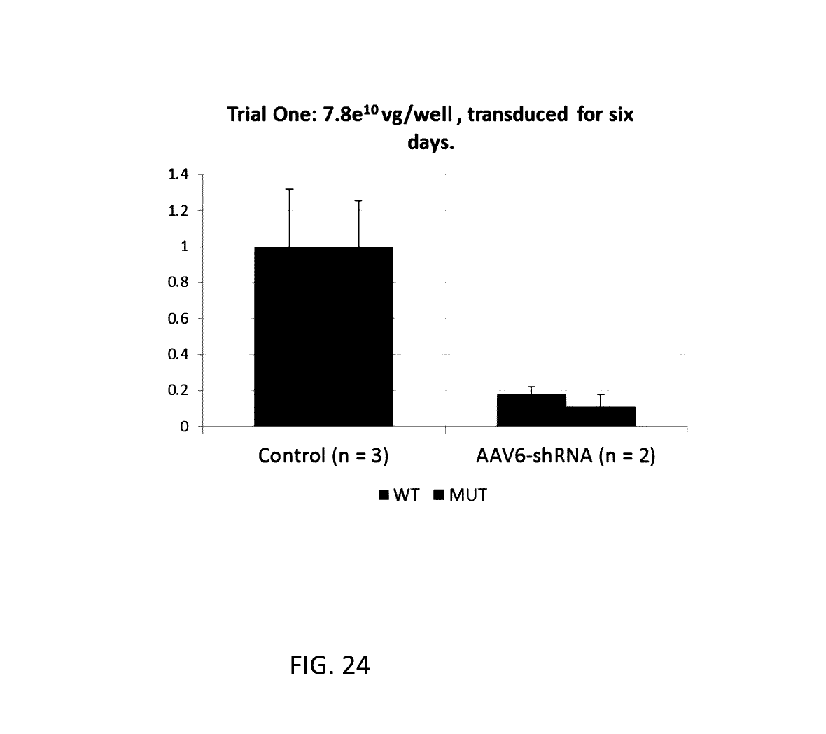

FIG. 24 show results that indicate fold change in wild type (WT) and mutant (MUT) MYH7 alleles. As shown, AAV6-shRNA-transduced-cell expression of each MYH7 allele is normalized to control expression. WT p-value=0.0408. MUT p-value=0.0199. Both the wild type and the mutant allele are significantly decreased. Error bars are standard deviation between transduced wells.

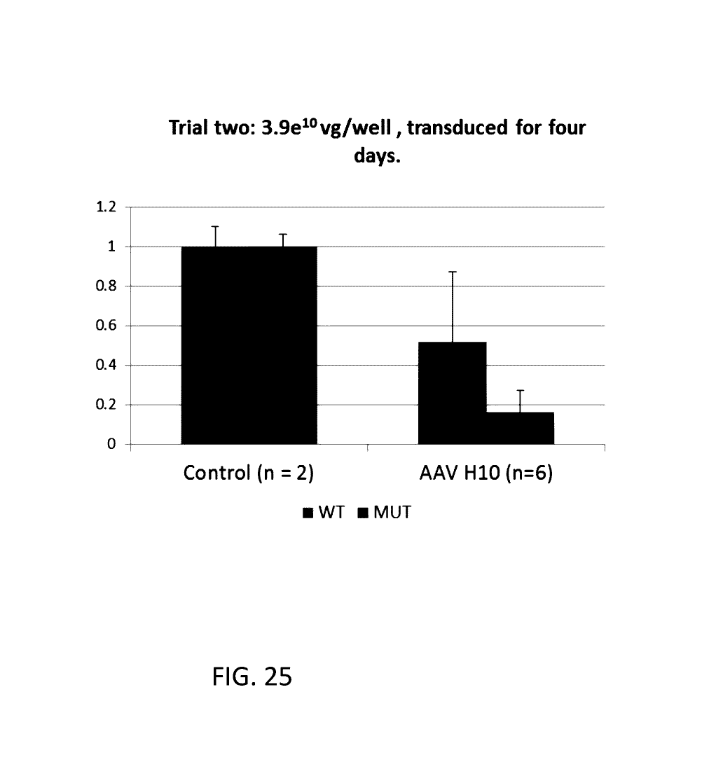

FIG. 25 show results that indicate fold change in wild type (WT) and mutant (MUT) MYH7 alleles. AAV6-shRNA-transduced-cell expression of each MYH7 allele is normalized to control expression. Samples with an 18S Ct value above 17 for the wild type allele QPCR reaction were removed. Samples with any “Undetermined” Ct values were also removed. WT p-value=0.1207. MUT p-value<0.0001. Error bars are standard deviation between transduced wells. The mutant allele is significantly reduced while wild type allele is not. Trial two shows the potential of allele-specific shRNAs delivered by AAV vectors to specifically silence a mutant allele.

It should be appreciated by those skilled in the art that the specific embodiments disclosed herein may be readily utilized as a basis for modifying or designing other embodiments. It should also be appreciated by those skilled in the art that such modifications do not depart from the scope of the invention as set forth in the appended claims. For example, variations to the methods can include changes that may improve the accuracy or flexibility of the disclosed methods.

1-40. (canceled)

41. A method of treatment, comprising:
having a human subject with a single nucleotide variant adenosine in the genetic code of at least one allele of the Myosin Light Chain 2 (MYL2) gene that results in a mutation of MYL2 proteins, wherein the mutation is a lysine at amino-acid position 47; and
administering an RNA-interference nucleic-acid therapeutic to the human subject, wherein the RNA-interference nucleic-acid therapeutic comprises a sequence that is substantially complimentary to a sequence of any one of the Seq. ID Nos. 137-139.

42. The method of claim 41, wherein the single nucleotide variant adenosine results with the human subject having hypertrophic cardiomyopathy.

43. The method of claim 41, wherein the RNA-interference nucleic-acid therapeutic downregulates RNA expression of at least one allele with the single nucleotide variant adenosine in the genetic code of the Myosin Light Chain 2 (MYL2) gene that results in said mutation of MYL2 proteins.

44. The method of claim 43, wherein the RNA-interference nucleic-acid therapeutic does not downregulate RNA expression of a healthy allele of the Myosin Light Chain 2 (MYL2) gene more than twenty percent;
wherein the healthy allele has a cytosine in the genetic code that results in an asparagine at amino-acid position 47.

45. The method of claim 41, wherein the RNA-interference nucleic-acid therapeutic is a single-stranded antisense oligonucleotide.

46. The method of claim 41, wherein the RNA-interference nucleic-acid therapeutic is a double-stranded small interfering RNA.

47. The method of claim 46, wherein the double-stranded small interfering RNA incorporates at least one nucleic base having a 2′-O-methyl modification.

48. The method of claim 41, wherein the RNA-interference nucleic-acid therapeutic is a short-hairpin RNA.

49. The method of claim 48, wherein the short-hairpin RNA is expressed from an expression vector.

50. The method of claim 49, wherein the expression vector is contained within a viral vector.

51. The method of claim 50, wherein the viral vector is an adeno-associated virus.

52. The method of claim 48, wherein the short-hairpin RNA sequence any one of Seq. ID Nos. 129-131.

53. A method of treatment, comprising:
having a human subject with a single nucleotide variant adenosine in the genetic code of at least one allele of the Myosin Heavy Chain 7 (MYH7) gene that results in a mutation of MYH7 proteins, wherein the mutation is a glutamine at amino-acid position 403; and
administering an RNA-interference nucleic-acid therapeutic to the human subject, wherein the RNA-interference nucleic-acid therapeutic comprises a sequence that is substantially complimentary to a sequence of either one of Seq. ID No. 53 and Seq. ID No. 54.

54. The method of claim 53, wherein the single nucleotide variant adenosine results with the human subject having hypertrophic cardiomyopathy.

55. The method of claim 53, wherein the RNA-interference nucleic-acid therapeutic downregulates RNA expression of at least one allele with the single nucleotide variant adenosine in the genetic code of the Myosin Heavy Chain 7 (MYH7) gene that results in said mutation of MYH7 proteins.

56. The method of claim 55, wherein the RNA-interference nucleic-acid therapeutic does not downregulate RNA expression of a healthy allele of the Myosin Heavy Chain 7 (MYH7) gene more than twenty percent;
wherein the healthy allele has a guanine in the genetic code that results in an arginine at amino-acid position 403.

57. The method of claim 53, wherein the RNA-interference nucleic-acid therapeutic is a single-stranded antisense oligonucleotide.

58. The method of claim 53, wherein the RNA-interference nucleic-acid therapeutic is a double-stranded small interfering RNA.

59. The method of claim 58, wherein the double-stranded small interfering RNA incorporates at least one nucleic base having a 2′-O-methyl modification.

60. The method of claim 53, wherein the RNA-interference nucleic-acid therapeutic is a short-hairpin RNA.

61. The method of claim 60, wherein the short-hairpin RNA is expressed from an expression vector.

62. The method of claim 61, wherein the expression vector is contained within a viral vector.

63. The method of claim 62, wherein the viral vector is an adeno-associated virus.

64. The method of claim 60, wherein the short-hairpin RNA sequence is either one of Seq. ID No. 132 and Seq. ID No. 133.

65. A therapeutic comprising an artificial nucleic-acid oligomer, wherein nineteen bases of the artificial nucleic-acid oligomer are substantially complementary to any one sequence of Seq. ID Nos. 137-139.

66. The therapeutic of claim 65, wherein the artificial nucleic-acid oligomer reduces RNA expression of a Myosin Light Chain 2 (MYL2) gene within a human cell;
wherein the MYL2 RNA has a single nucleotide variant adenosine in the genetic code that results in a mutation of MYL2 proteins, wherein the mutation is a lysine at amino-acid position 47; and
wherein the human cell expresses the MYL2 RNA having said single nucleotide variant adenosine.

67. The therapeutic of claim 65, wherein the artificial nucleic-acid oligomer is a single-stranded antisense oligomer.

68. The therapeutic of claim 65, wherein the artificial nucleic acid oligomer is a double-stranded small interfering RNA.

69. The therapeutic of claim 68, wherein the double-stranded small interfering RNA incorporates at least one nucleic base having a 2′-O-methyl modification.

70. The therapeutic of claim 65, wherein the artificial nucleic-acid oligomer is a short hairpin RNA.

71. The therapeutic of claim 70, wherein the short hairpin RNA is expressed from a viral vector.

72. The therapeutic of claim 71, wherein the viral vector is an adeno-associated virus.

73. The therapeutic of claim 70, wherein the short hairpin RNA sequence is any one of Seq. ID Nos. 129-131.

74. A therapeutic comprising an artificial nucleic-acid oligomer, wherein nineteen bases of the artificial nucleic-acid oligomer are substantially complementary to either one sequence of Seq. ID No. 53 and Seq. ID No. 54.

75. The therapeutic of claim 64, wherein the artificial nucleic-acid oligomer reduces RNA expression of a Myosin Heavy Chain 7 (MYH7) gene within a human cell;
wherein the MYH7 RNA has a single nucleotide variant adenosine in the genetic code that results in a mutation of MYH7 proteins, wherein the mutation is a glutamine at amino-acid position 403; and
wherein the human cell expresses the MYH7 RNA having said single nucleotide variant adenosine.

76. The therapeutic of claim 74, wherein the artificial nucleic-acid oligomer is a single-stranded antisense oligomer.

77. The therapeutic of claim 74, wherein the artificial nucleic acid oligomer is a double-stranded small interfering RNA.

78. The therapeutic of claim 77, wherein the double-stranded small interfering RNA incorporates at least one nucleic base having a 2′-O-methyl modification.

79. The therapeutic of claim 74, wherein the artificial nucleic-acid oligomer is a short hairpin RNA.

80. The therapeutic of claim 79, wherein the short hairpin RNA is expressed from a viral vector.

81. The therapeutic of claim 80, wherein the viral vector is an adeno-associated virus.

82. The therapeutic of claim 79, wherein the short hairpin RNA sequence is either one of Seq. ID No. 132 and Seq. ID No. 133.

83. A method of RNAi therapeutic design, comprising:
implementing a Variant Call Format (VCF) file on a computer, wherein the VCF file contains at least one single nucleotide polymorphic (SNP) target of interest, wherein each SNP target of interest corresponds with a first allele of a human gene, wherein the first allele has a mutation that correlates with a medical disorder, and wherein the second allele is healthy;
acquiring a DNA sequence for each SNP target of interest, wherein the DNA sequence corresponds to the reference genome sequence that immediately surrounds each SNP target of interest;
generating all possible short-hairpin RNAs (shRNAs) and antisense oligo sequences (ASOs) for each SNP target of interest; and
ranking the shRNAs and the ASOs for each SNP target of interest, wherein the shRNAs and the ASOs are ranked based on predetermined qualities from which a list of candidate shRNAs and ASOs can be identified for each SNP target of interest.


SEQUENCE LISTING (125 sequences)

SEQ ID NO: 1 - DNA, 12 bp, Artificial Sequence
  shRNA loop 12
cttcctgtcaga

SEQ ID NO: 2 - RNA, 63 nt, Homo sapiens
  CDS (1)..(63)
acuaucauggaccagaacagggauggcuucauugacaagaacgaucugagagacaccuuugcu

SEQ ID NO: 3 - protein, 21 aa, Homo sapiens
TIMDQNRDGFIDKNDLRDTFA

SEQ ID NO: 4 - RNA, 63 nt, Homo sapiens
  CDS (1)..(63)
acuaucauggaccagaacagggauggcuucauugacaagaaagaucugagagacaccuuugcu

SEQ ID NO: 5 - protein, 21 aa, Homo sapiens
TIMDQNRDGFIDKKDLRDTFA

SEQ ID NO: 6 - RNA, 19 nt, Artificial Sequence
  MYL2 wild-type siRNA
cuucauugacaagaacgau

SEQ ID NO: 7 - RNA, 19 nt, Artificial Sequence
  MYL2-N47K siRNA M2 sense strand
aagaucugagagacaccuu

SEQ ID NO: 8 - RNA, 19 nt, Artificial Sequence
  MYL2-N47K siRNA M3 sense strand
aaagaucugcgagacaccu

SEQ ID NO: 9 - RNA, 19 nt, Artificial Sequence
  MYL2-N47K siRNA M4 sense strand
gaaagaucugagagacacc

SEQ ID NO: 10 - RNA, 19 nt, Artificial Sequence
  MYL2-N47K siRNA M5 sense strand
agaaagaucugagagacac

SEQ ID NO: 11 - RNA, 19 nt, Artificial Sequence
  MYL2-N47K siRNA M6 sense strand
aagaaagaucugagagaca

SEQ ID NO: 12 - RNA, 19 nt, Artificial Sequence
  MYL2-N47K siRNA M7 sense strand
caagaaagaucugagagac

SEQ ID NO: 13 - RNA, 19 nt, Artificial Sequence
  MYL2-N47K siRNA M8 sense strand
acaagaaagaucugagaga

SEQ ID NO: 14 - RNA, 19 nt, Artificial Sequence
  MYL2-N47K siRNA M9 sense strand
gacaagaaagaucugagag

SEQ ID NO: 15 - RNA, 19 nt, Artificial Sequence
  MYL2-N47K siRNA M10 sense strand
ugacaagaaagaucugaga

SEQ ID NO: 16 - RNA, 19 nt, Artificial Sequence
  MYL2-N47K siRNA M11 sense strand
uugacaagaaagaucugag

SEQ ID NO: 17 - RNA, 19 nt, Artificial Sequence
  MYL2-N47K siRNA M12 sense strand
auugacaagaaagaucuga

SEQ ID NO: 18 - RNA, 19 nt, Artificial Sequence
  MYL2-N47K siRNA M13 sense strand
cauugacaagaaagaucug

SEQ ID NO: 19 - RNA, 19 nt, Artificial Sequence
  MYL2-N47K siRNA M14 sense strand
ucauugacaagaaagaucu

SEQ ID NO: 20 - RNA, 19 nt, Artificial Sequence
  MYL2-N47K siRNA M15 sense strand
uucauugacaagaaagauc

SEQ ID NO: 21 - RNA, 19 nt, Artificial Sequence
  MYL2-N47K siRNA M16 sense strand
cuucauugacaagaaagau

SEQ ID NO: 22 - RNA, 19 nt, Artificial Sequence
  MYL2-N47K siRNA M17 sense strand
gcuucauugacaagaaaga

SEQ ID NO: 23 - RNA, 19 nt, Artificial Sequence
  MYL2-N47K siRNA M18 sense strand
ggcuucauugacaagaaag

SEQ ID NO: 24 - RNA, 19 nt, Artificial Sequence
  MYL2-N47K siRNA M19 sense strand
uggcuucauugacaagaaa

SEQ ID NO: 25 - DNA, 21 bp, Artificial Sequence
  M20_MYL2-47K siRNA antisense strand
ttucuuucuagacucucugug

SEQ ID NO: 26 - DNA, 21 bp, Artificial Sequence
  M21_MYL2-47K siRNA sense strand
agaaagaucugagagacautt

SEQ ID NO: 27 - DNA, 21 bp, Artificial Sequence
  M21_MYL2-47K siRNA antisense strand
uuucuuucuagacucucugug

SEQ ID NO: 28 - DNA, 21 bp, Artificial Sequence
  M22_MYL2-47K siRNA sense strand
agaaagaucugagagacautt

SEQ ID NO: 29 - DNA, 21 bp, Artificial Sequence
  M22_MYL2-47K siRNA antisense strand
ttucuuucuagacucucugug

SEQ ID NO: 30 - DNA, 20 bp, Artificial Sequence
  M23_MYL2-47K siRNA antisense strand
ttguucuuucuagacucucu

SEQ ID NO: 31 - DNA, 21 bp, Artificial Sequence
  M24_MYL2-47K siRNA sense strand
caagaaagaucugagagautt

SEQ ID NO: 32 - DNA, 21 bp, Artificial Sequence
  M24_MYL2-47K siRNA antisense strand
ttguucuuucuagacucucug

SEQ ID NO: 33 - DNA, 21 bp, Artificial Sequence
  M25_MYL2-47K siRNA sense strand
caagaaagaucugagagautt

SEQ ID NO: 34 - DNA, 22 bp, Artificial Sequence
  M25_MYL2-47K siRNA antisense strand
uuuguucuuucuagacucucug

SEQ ID NO: 35 - DNA, 48 bp, Artificial Sequence
  M5 8-loop shRNA
agaaagaucugagagacacgaagcttggugucucucagaucuuucuuu

SEQ ID NO: 36 - DNA, 48 bp, Artificial Sequence
  M6 8-loop shRNA
aagaaagaucugagagacagaagcttgugucucucagaucuuucuuuu

SEQ ID NO: 37 - DNA, 48 bp, Artificial Sequence
  M7 8-loop shRNA
caagaaagaucugagagacgaagcttggucucucagaucuuucuuguu

SEQ ID NO: 38 - DNA, 48 bp, Artificial Sequence
  M8 8-loop shRNA
acaagaaagaucugagagagaagcttgucucucagaucuuucuuguuu

SEQ ID NO: 39 - DNA, 48 bp, Artificial Sequence
  M9 8-loop shRNA
gacaagaaagaucugagaggaagcttgcucucagaucuuucuugucuu

SEQ ID NO: 40 - DNA, 60 bp, Homo sapiens
  CDS (1)..(60)
ucagccgaccugcucaaggggcugugccacccucgggugaaagugggcaaugaguacgtc

SEQ ID NO: 41 - protein, 20 aa, Homo sapiens
SADLLKGLCHPRVKVGNEYV

SEQ ID NO: 42 - DNA, 60 bp, Homo sapiens
  CDS (1)..(60)
ucagccgaccugcucaaggggcugugccacccucaggugaaagugggcaaugaguacgtc

SEQ ID NO: 43 - protein, 20 aa, Homo sapiens
SADLLKGLCHPQVKVGNEYV

SEQ ID NO: 44 - RNA, 19 nt, Artificial Sequence
  MYH7-R403Q siRNA H1 sense strand
aggugaaagugggcaauga

SEQ ID NO: 45 - RNA, 19 nt, Artificial Sequence
  MYH7-R403Q siRNA H2 sense strand
caggugaaagugggcaaug

SEQ ID NO: 46 - RNA, 19 nt, Artificial Sequence
  MYH7-R403Q siRNA H3 sense strand
ucaggugaaagugggcaau

SEQ ID NO: 47 - RNA, 19 nt, Artificial Sequence
  MYH7-R403Q siRNA H4 sense strand
cucaggugaaagugggcaa

SEQ ID NO: 48 - RNA, 19 nt, Artificial Sequence
  MYH7-R403Q siRNA H5 sense strand
ccucaggugaaagugggca

SEQ ID NO: 49 - RNA, 19 nt, Artificial Sequence
  MYH7-R403Q siRNA H6 sense strand
cccucaggugaaagugggc

SEQ ID NO: 50 - RNA, 19 nt, Artificial Sequence
  MYH7-R403Q siRNA H7 sense strand
acccucaggugaaaguggg

SEQ ID NO: 51 - RNA, 19 nt, Artificial Sequence
  MYH7-R403Q siRNA H8 sense strand
cacccucaggugaaagugg

SEQ ID NO: 52 - RNA, 19 nt, Artificial Sequence
  MYH7-R403Q siRNA H9 sense strand
ccacccucaggugaaagug

SEQ ID NO: 53 - RNA, 19 nt, Artificial Sequence
  MYH7-R403Q siRNA H10 sense strand
gccacccucaggugaaagu

SEQ ID NO: 54 - RNA, 19 nt, Artificial Sequence
  MYH7-R403Q siRNA H11 sense strand
ugccacccucaggugaaag

SEQ ID NO: 55 - RNA, 19 nt, Artificial Sequence
  MYH7-R403Q siRNA H12 sense strand
gugccacccucaggugaaa

SEQ ID NO: 56 - RNA, 19 nt, Artificial Sequence
  MYH7-R403Q siRNA H13 sense strand
ugugccacccucaggugaa

SEQ ID NO: 57 - RNA, 19 nt, Artificial Sequence
  MYH7-R403Q siRNA H14 sense strand
cugugccacccucagguga

SEQ ID NO: 58 - RNA, 19 nt, Artificial Sequence
  MYH7-R403Q siRNA H15 sense strand
gcugugccacccucaggug

SEQ ID NO: 59 - RNA, 19 nt, Artificial Sequence
  MYH7-R403Q siRNA H16 sense strand
ggcugugccacccucaggu

SEQ ID NO: 60 - RNA, 19 nt, Artificial Sequence
  MYH7-R403Q siRNA H17 sense strand
gggcugugccacccucagg

SEQ ID NO: 61 - RNA, 19 nt, Artificial Sequence
  MYH7-R403Q siRNA H18 sense strand
ggggcugugccacccucag

SEQ ID NO: 62 - RNA, 19 nt, Artificial Sequence
  MYH7-R403Q siRNA H19 sense strand
aggggcugugccacccuca

SEQ ID NO: 63 - DNA, 48 bp, Artificial Sequence
  MYH7-R403Q shRNA H1.8L
aggugaaagugggcaaugagaagcttgucauugcccacuuucaccuuu

SEQ ID NO: 64 - DNA, 48 bp, Artificial Sequence
  MYH7-R403Q shRNA H10.8L
gccucccucaggugaaagugaagcttgacuuucaccugagggaggcuu

SEQ ID NO: 65 - DNA, 48 bp, Artificial Sequence
  MYH7-R403Q shRNA H11.8L
tgccucccucaggugaaaggaagcttgcuuucaccugagggaggcauu

SEQ ID NO: 66 - DNA, 48 bp, Artificial Sequence
  MYH7-R403Q shRNA H12.8L
gtgccucccucaggugaaagaagcttguuucaccugagggaggcacuu

SEQ ID NO: 67 - DNA, 48 bp, Artificial Sequence
  MYH7-R403Q shRNA H13.8L
ugtgccucccucaggugaagaagcttguucaccugagggaggcacauu

SEQ ID NO: 68 - DNA, 39 bp, Artificial Sequence
  sense primer for MYL2-47 mutagenesis
atggcttcattgacaagaaagatctgagagacacctttg

SEQ ID NO: 69 - DNA, 39 bp, Artificial Sequence
  antisense primer for MYL2-47 mutagenesis
caaaggtgtctctcagatctttcttgtcaatgaagccat

SEQ ID NO: 70 - DNA, 30 bp, Artificial Sequence
  forward PCR primer
gacggtaccccatgtacctcatggggctga

SEQ ID NO: 71 - DNA, 31 bp, Artificial Sequence
  reverse PCR primer
gcgaccggttgctggacattctgccccttgg

SEQ ID NO: 72 - DNA, 19 bp, Artificial Sequence
  sense primer for MYH7 mutagenesis
caccctcaggtgaaagtgg

SEQ ID NO: 73 - DNA, 19 bp, Artificial Sequence
  antisense primer for MYH7 mutagenesis
cccactttcacctgagggt

SEQ ID NO: 74 - DNA, 25 bp, Artificial Sequence
  MYH7_Hind III forward PCR primer
aagcttatgggagattcggagatgg

SEQ ID NO: 75 - DNA, 24 bp, Artificial Sequence
  MYH7_AgeI reverse PCR primer
accggtacaaacatgtggtggttg

SEQ ID NO: 76 - DNA, 24 bp, Artificial Sequence
  MYL2-forward PCR primer
aagaaagcaaagaagagagccggg

SEQ ID NO: 77 - DNA, 24 bp, Artificial Sequence
  MYL2-reverse PCR primer
tgtgcaccaggttcttgtagtcca

SEQ ID NO: 78 - DNA, 20 bp, Artificial Sequence
  P3 (Fwd-20mer) primer
atggcttcattgacaagaaa

SEQ ID NO: 79 - DNA, 19 bp, Artificial Sequence
  P4 (Fwd-19mer) primer
tggcttcattgacaagaaa

SEQ ID NO: 80 - DNA, 18 bp, Artificial Sequence
  P5 (Fwd-18mer) primer
ggcttcattgacaagaaa

SEQ ID NO: 81 - DNA, 22 bp, Artificial Sequence
  PWT-Fwd primer
ggatggcttcattgacaagaac

SEQ ID NO: 82 - DNA, 22 bp, Artificial Sequence
  PWT-Rev primer
ttcctcagggtccgctccctta

SEQ ID NO: 83 - DNA, 19 bp, Artificial Sequence
  B1 (wild type blocker 1)
tgacaagaacgatctgaga

SEQ ID NO: 84 - DNA, 20 bp, Artificial Sequence
  B3 (Mutant Blocker 3)
atggcttcattgacaagaac

SEQ ID NO: 85 - DNA, 19 bp, Artificial Sequence
  B4 (Mutant Blocker 4)
tggcttcattgacaagaac

SEQ ID NO: 86 - DNA, 18 bp, Artificial Sequence
  B5 (Mutant Blocker 5)
ggcttcattgacaagaac

SEQ ID NO: 87 - DNA, 26 bp, Artificial Sequence
  MYL2 Taqman Probe
tggatgaaatgatcaaggaggctccg

SEQ ID NO: 88 - RNA, 21 nt, Artificial Sequence
  W16_MYL2-47K siRNA antisense strand
ccgaaguaacuguucuugcua

SEQ ID NO: 89 - RNA, 21 nt, Artificial Sequence
  M2_MYL2-47K siRNA antisense strand
cuuucuagacucucuguggaa

SEQ ID NO: 90 - RNA, 21 nt, Artificial Sequence
  M3_MYL2-47K siRNA antisense strand
ucuuucuagacucucugugga

SEQ ID NO: 91 - RNA, 21 nt, Artificial Sequence
  M4_MYL2-47K siRNA antisense strand
uucuuucuagacucucugugg

SEQ ID NO: 92 - RNA, 21 nt, Artificial Sequence
  M6_MYL2-47K siRNA antisense strand
uguucuuucuagacucucugu

SEQ ID NO: 93 - RNA, 21 nt, Artificial Sequence
  M7_MYL2-47K siRNA antisense strand
cuguucuuucuagacucucug

SEQ ID NO: 94 - RNA, 21 nt, Artificial Sequence
  M8_MYL2-47K siRNA antisense strand
acuguucuuucuagacucucu

SEQ ID NO: 95 - RNA, 21 nt, Artificial Sequence
  M9_MYL2-47K siRNA antisense strand
aacuguucuuucuagacucuc

SEQ ID NO: 96 - RNA, 21 nt, Artificial Sequence
  M10_MYL2-47K siRNA antisense strand
uaacuguucuuucuagacucu

SEQ ID NO: 97 - RNA, 21 nt, Artificial Sequence
  M11_MYL2-47K siRNA antisense strand
guaacuguucuuucuagacuc

SEQ ID NO: 98 - RNA, 21 nt, Artificial Sequence
  M12_MYL2-47K siRNA antisense strand
aguaacuguucuuucuagacu

SEQ ID NO: 99 - RNA, 21 nt, Artificial Sequence
  M13_MYL2-47K siRNA antisense strand
aaguaacuguucuuucuagac

SEQ ID NO: 100 - RNA, 21 nt, Artificial Sequence
  M14_MYL2-47K siRNA antisense strand
gaaguaacuguucuuucuaga

SEQ ID NO: 101 - RNA, 21 nt, Artificial Sequence
  M15_MYL2-47K siRNA antisense strand
cgaaguaacuguucuuucuag

SEQ ID NO: 102 - RNA, 21 nt, Artificial Sequence
  M16_MYL2-47K siRNA antisense strand
ccgaaguaacuguucuuucua

SEQ ID NO: 103 - RNA, 21 nt, Artificial Sequence
  M17_MYL2-47K siRNA antisense strand
accgaaguaacuguucuuucu

SEQ ID NO: 104 - RNA, 21 nt, Artificial Sequence
  M18_MYL2-47K siRNA antisense strand
uaccgaaguaacuguucuuuc

SEQ ID NO: 105 - RNA, 21 nt, Artificial Sequence
  M19_MYL2-47K siRNA antisense strand
cuaccgaaguaacuguucuuu

SEQ ID NO: 106 - RNA, 19 nt, Artificial Sequence
  M7-S2 MYL2-47K siRNA sense strand
caagaaaguucugagagac

SEQ ID NO: 107 - RNA, 21 nt, Artificial Sequence
  M7-S2 MYL2-47K siRNA antisense strand
cuguucuuucuagacucucug

SEQ ID NO: 108 - RNA, 19 nt, Artificial Sequence
  M7-S4A MYL2-47K siRNA sense strand
caagaaagauaugagagac

SEQ ID NO: 109 - RNA, 21 nt, Artificial Sequence
  M7-S4A MYL2-47K siRNA antisense strand
cuguucuuucuagacucucug

SEQ ID NO: 110 - RNA, 19 nt, Artificial Sequence
  M7-S4G MYL2-47K siRNA sense strand
caagaaagaugugagagac

SEQ ID NO: 111 - RNA, 21 nt, Artificial Sequence
  M7-S4G MYL2-47K siRNA antisense strand
cuguucuuucuagacucucug

SEQ ID NO: 112 - RNA, 19 nt, Artificial Sequence
  M7-S4U MYL2-47K siRNA sense strand
caagaaagauuugagagac

SEQ ID NO: 113 - RNA, 21 nt, Artificial Sequence
  M7-S4U MYL2-47K siRNA antisense strand
cuguucuuucuagacucucug

SEQ ID NO: 114 - RNA, 19 nt, Artificial Sequence
  M7-A2 MYL2-47K siRNA sense strand
caagaaagaucugagagac

SEQ ID NO: 115 - RNA, 21 nt, Artificial Sequence
  M7-A2 MYL2-47K siRNA antisense strand
cuguucuuucaagacucucug

SEQ ID NO: 116 - RNA, 19 nt, Artificial Sequence
  M7-A4 MYL2-47K siRNA sense strand
caagaaagaucugagagac

SEQ ID NO: 117 - RNA, 21 nt, Artificial Sequence
  M7-A4 MYL2-47K siRNA antisense strand
cuguucuuucuaaacucucug

SEQ ID NO: 118 - RNA, 19 nt, Artificial Sequence
  M7-A4U MYL2-47K siRNA sense strand
caagaaagaucugagagac

SEQ ID NO: 119 - RNA, 21 nt, Artificial Sequence
  M7-A4U MYL2-47K siRNA antisense strand
cuguucuuucuauacucucug

SEQ ID NO: 120 - DNA, 4842 bp, Artificial Sequence
  pAAV H1-M7.8L RSV-cerulean
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctctctagaatgcaggggggggggggggggggggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgatatcggcctcgagtgatcaaaaaaccaacacacgcttccaatgaaaataaacgatcctttattgctagcctcttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagtgggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtacgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacctgtggccgtttacgtcgccgtccagctcgaccaggatgggcaccaccccggtgaacagctcctcgcccttgctcaccatggtggcgaccggtggatcccgggcccgcggtacccagcttggaggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatccttaccatgttgcaagactaccaagagtatttgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccatacggatctgcgatgataagctgtcaaacatgagaattggtcgaccgcaaaaaacaagaaagatctgagagaccaagcttcgtctctcagatctttcttgcggtgtaccgtggtctcatacagaacttataagattcccaaatccaaagacatttcacgtttatggtgatttcccagaacacatagcgacatgcaaatatgggcgcgccgatatcagatctgggaaaccagatgatggaggtacccactccctctatgcgcgctcgctcactcactcggccctgccggccagaggccggcagtctggagacctttggtctccagggccgagtgagtgagcgagcgcgcatagagggagtgggtaggacgcgtcctgcaggatgcatactagtggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 121 - DNA, 4796 bp, Artificial Sequence
  pAAV RSV-cerulean
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctctctagaatgcaggggggggggggggggggggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgatatcggcctcgagtgatcaaaaaaccaacacacgcttccaatgaaaataaacgatcctttattgctagcctcttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcggatcttgaagtgggccttgatgccgttcttctgcttgtcggcggtgatatagacgttgtcgctgatggcgttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtacgctcctggacgtagccttcgggcatggcggacttgaagaagtcgtgctgcttcatgtggtcggggtagcgggcgaagcactgcacgccccaggtcagggtggtcacgagggtgggccagggcacgggcagcttgccggtggtgcagatgaacttcagggtcagcttgccgtaggtggcatcgccctcgccctcgccggacacgctgaacctgtggccgtttacgtcgccgtccagctcgaccaggatgggcaccaccccggtgaacagctcctcgcccttgctcaccatggtggcgaccggtggatcccgggcccgcggtacccagcttggaggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatccttaccatgttgcaagactaccaagagtatttgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccatacggatctgcgatgataagctgtcaaacatgagaattggtcgaccgcaaaaaacggtgtaccgtggtctcatacagaacttataagattcccaaatccaaagacatttcacgtttatggtgatttcccagaacacatagcgacatgcaaatatgggcgcgccgatatcagatctgggaaaccagatgatggaggtacccactccctctatgcgcgctcgctcactcactcggccctgccggccagaggccggcagtctggagacctttggtctccagggccgagtgagtgagcgagcgcgcatagagggagtgggtaggacgcgtcctgcaggatgcatactagtggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 122 - DNA, 4931 bp, Artificial Sequence
  pAAV-CBA-Fluc
gggcgaattggccaagtcggccgagctcgaattcgtcgacctcgaggcctcggaggattacaatagctaagaatttcgtcatcgctgaatacagttacattttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccatttggatccgggccctctagatgcggccgcatgcataagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccgcaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 123 - DNA, 11184 bp, Artificial Sequence
  pRSV eGFP-T2A-Fluc2
agcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttacagccggacgtgcaactccgctggtggtctaacgtcggttaggtcccttgaatcacgggacatatgttggtgttggaggtttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatcgatatcggcgcgcccatatttgcatgtcgctatgtgttctgggaaatcaccataaacgtgaaatgtctttggatttgggaatcttataagttctgtatgagaccacggtacaccgttttgcggtcgaccaattctcatgtttgacagcttatcagcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttacagccggacgtgcaactccgctggtggtctaacgtcggttaggtcccttgaatcacgggacatatgttggtgttggaggtttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatcgatatcggcgcgcccatatttgcatgtcgctatgtgttctgggaaatcaccataaacgtgaaatgtctttggatttgggaatcttataagttctgtatgagaccacggtacaccggccaccctcaggtgaaagtgaagcttgactttcacctgagggtggcttttttgcggtcgaccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcagaggaagtctgctaacatgcggtgacgtcgaggagaatcctggcccaatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagctttccatatttgcctttcattgcacactcttcaccccagctaataattccagttaagaaactggtcccttccacttcagtaacatggggtcccccactatctccttgacatgaatctctacctccttcatggaagccagcacagaacatgttgttatagatggtgaactttgtagatcgaagacatgtggctcggtcaacaagtggaactctaaggtactgaagaactaaagctgatctccctttgtggaagactcttccccagccacttacatagccagatccaaatttgaggaagatgttcgtgtattccatatgtttacgtcacgactccacccctccaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 124 - DNA, 7131 bp, Artificial Sequence
  pAAV-CBA-Fluc-H1p-H11.8L
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttaaaaactagtccgcggaattctcgagctagcggccgcggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactcatctggtttcccagatctgatatcggcgcgcccatatttgcatgtcgctatgtgttctgggaaatcaccataaacgtgaaatgtctttggatttgggaatcttataagttctgtatgagaccacggtacaccgtgccaccctcaggtgaaaggaagcttgctttcacctgagggtggcattttttgcggtcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 125 - DNA, 7515 bp, Artificial Sequence
  pRSV eGFP-T2A-Fluc2-H10.8L
agcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttacagccggacgtgcaactccgctggtggtctaacgtcggttaggtcccttgaatcacgggacatatgttggtgttggaggtttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatcgatatcggcgcgcccatatttgcatgtcgctatgtgttctgggaaatcaccataaacgtgaaatgtctttggatttgggaatcttataagttctgtatgagaccacggtacaccggccaccctcaggtgaaagtgaagcttgactttcacctgagggtggcttttttgcggtcgaccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcagaggaagtctgctaacatgcggtgacgtcgaggagaatcctggcccaatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaagctttccatatttgcctttcattgcacactcttcaccccagctaataattccagttaagaaactggtcccttccacttcagtaacatggggtcccccactatctccttgacatgaatctctacctccttcatggaagccagcacagaacatgttgttatagatggtgaactttgtagatcgaagacatgtggctcggtcaacaagtggaactctaaggtactgaagaactaaagctgatctccctttgtggaagactcttccccagccacttacatagccagatccaaatttgaggaagatgttcgtgtattccatatgtttacgtcacgactccacccctccaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa